(19) United States
(12) Patent Application Publication
(10) Pub. No.: US 2018/0286508 A1
(43) Pub. Date: Oct. 4, 2018

(54) DETERMINATION OF FAVORABLE DATE(S) FOR THERAPEUTIC TREATMENT DELIVERY

(71) Applicant: Mayo Foundation for Medical Education and Research, Rochester, MN (US)

(72) Inventors: Alexey A. Leontovich, Rochester, MN (US); Leonid V. Ivanov, Grinnell, IA (US); Svetomir N. Markovic, Rochester, MN (US)

(73) Assignee: Mayo Foundation for Medical Education and Research, Rochester, MN (US)

(21) Appl. No.: 15/765,702

(22) PCT Filed: Aug. 24, 2016
(86) PCT No.: PCT/US2016/048440

§ 371 Date: Apr. 3, 2018

Related U.S. Application Data
(60) Provisional application No. 62/239,355, filed on Oct. 9, 2015.

(51) Int. Cl. G16H 20/40; G16H 10/60; G06Q 10/10; G01N 33/50; G01N 33/68

(52) U.S. Cl. CPC G16H 20/40; G16H 10/60; G01N 33/6866; G01N 33/5047; G01N 33/6869; G06Q 10/1095

(57) ABSTRACT

A system and method determines one or more favorable dates for delivery of therapeutic treatment to a specific patient based on the “state” of one or more biological variables of the patient on the proposed treatment date(s). The “state” of the biological variables refers to whether the concentration of the biological variable is greater than a threshold value (state=HIGH or UP) on the proposed date of treatment or less than a threshold value (state=LOW or DOWN) on the proposed date of treatment. The biological variables may include lymphocytes and/or monocytes. A system and method may also determine one or more favorable dates for delivery of therapeutic treatment to patient based on a lymphocyte-to-monocyte ratio on the proposed treatment dates, and/or the “state” of the lymphocytes and monocytes on the proposed treatment dates.

### RELATED APPLICATION

This application is a 371 application of International Application No. PCT/US2016/048440, filed Aug. 24, 2016, which claims the benefit of U.S. Provisional Application No. 62/239,355, filed Oct. 9, 2015, the entire contents of each of which are incorporated herein by reference.

### TECHNICAL FIELD

The disclosure relates to planning of pharmaceutical treatment.

### BACKGROUND

Cancer refers to any one of a large number of diseases characterized by the development of abnormal cells that divide uncontrollably and have the ability to infiltrate and destroy normal body tissue. Skin cancer affects more people in the United States than any other malignancy, and malignant melanoma is particularly deadly due to its metastatic potential. Melanoma incidence has been increasing dramatically worldwide, and it is currently the sixth most common diagnosed cancer in developed countries, with the burden being carried mostly by fair-skinned populations. In the United States, the incidence of many common cancers, including breast, colon, or prostate cancer, has either remained steady, or declined over time, while melanoma incidence has steadily increased year after year. Signs and symptoms caused by cancer will vary depending on what part of the body is affected. Cancer treatments include surgery, chemotherapy, radiation therapy, stem cell transplant, immunotherapy, hormone therapy, and targeted drug delivery.

### SUMMARY

In general, the disclosure relates to planning delivery of chemotherapy or other pharmaceutical treatment. For example, systems and/or methods described herein analyze time-dependent fluctuations of at least one biological variable measured in blood samples of a patient and determine one or more favorable dates for delivery of pharmaceutical treatment to the patient.

In one example, the disclosure is directed to a method of identifying one or more favorable dates to deliver a pharmacological treatment to a patient, comprising: receiving data corresponding to concentrations of one or more biological variables in blood samples from the patient over an observed time period; for each of the biological variables, fitting a periodic function to the received data corresponding to the concentration of the biological variable in the blood samples of the patient; for each of the biological variables, extrapolating the corresponding fitted periodic function to a plurality of proposed treatment dates occurring subsequent to the observed time period; for each of the biological variables, determining a state of the corresponding fitted periodic function on each of the plurality of proposed treatment dates, wherein the state on a proposed treatment date for a first set of the biological variables is determined to be favorable if the fitted periodic function is greater than a threshold value associated with the biological variable in the first set of biological variables on the proposed treatment date, and wherein the state on a proposed treatment date for a second set of the biological variables is determined to be favorable if the fitted periodic function is less than a threshold value associated with the biological variable in the second set of biological variables on the proposed treatment date; and identifying at least one of the plurality of proposed treatment dates as a favorable date to deliver the pharmacological treatment to the patient based on the determined states of each of the biological variables on each of the plurality of proposed treatment dates.

The identified at least one favorable date to deliver the pharmacological treatment to the patient may correspond to one of the plurality of proposed treatment dates having a maximum number of the biological variables in a favorable state. The method may further include, for each of the biological variables, determining whether the extrapolated corresponding fitted periodic function is in an unfavorable state on each of the proposed treatment dates; and wherein the identified at least one favorable date to deliver the pharmacological treatment to the patient corresponds to one of the plurality of proposed treatment dates having a maximum number of the biological variables in a favorable state and a minimum number of the biological variables in an unfavorable state. The method may further include developing a treatment plan for the patient based on the identified at least one favorable date to deliver the pharmacological treatment to the patient. The method may further include delivering the pharmacological treatment to the patient on the identified at least one favorable date to deliver the pharmacological treatment to the patient. The first set of biological variables may include at least one lymphocyte sub-type and the second set of biological variables may include at least one monocyte sub-type. The first set of the biological variables may include at least one of CD3.4 and GRO and the second set of the biological variables may include at least one of IL-2, CD123.DR(DC2), CD11c/86, CD11c/14, TGFa, and IFNg. Fitting a periodic function to the received data corresponding to the concentration of the biological variable in the blood samples of the patient may include fitting the received data corresponding to the concentration of the biological variable in the blood samples of the patient to a sinusoidal function.

In another example, the disclosure is directed to a method of identifying one or more favorable dates to deliver a pharmacological treatment to a patient, comprising: receiving data corresponding to levels of one or more lymphocyte subtypes in blood samples from the patient over an observed time period; receiving data corresponding to levels of one or more monocyte subtypes in blood samples from the patient over the observed time period; for each of the lymphocyte subtypes, fitting a periodic function to the received data corresponding to the levels of the lymphocyte subtype in the blood samples of the patient, and extrapolating the fitted periodic function to a plurality of proposed treatment dates occurring subsequent to the observed time period; for each of the monocyte subtypes, fitting a periodic function to the received data corresponding to the levels of the monocyte subtype in the blood samples of the patient, and extrapolating the fitted periodic function to a plurality of proposed treatment dates occurring subsequent to the observed time period; determining a lymphocyte-to-monocyte ratio on each of the plurality of proposed treatment dates based on the extrapolated fitted periodic function for the lymphocyte subtype and the extrapolated fitted periodic function for the monocyte subtype; identifying at least one of the plurality of proposed treatment dates as a favorable date to deliver the pharmacological treatment to the patient, wherein the favorable date to deliver the pharmacological treatment to the patient corresponds to the proposed treatment date when the lymphocyte-to-monocyte ratio is at or near a maximum.

The method may further include delivering the pharmacological treatment to the patient on the at least one identified favorable date. The lymphocyte-to-monocyte ratio may include a ratio of the lymphocyte concentration to the monocyte concentration. The lymphocyte-to-monocyte ratio may include a ratio of the absolute lymphocyte count to the absolute monocyte count. The lymphocyte-to-monocyte ratio may be based on a defined set of one or more lymphocyte subtypes and a defined set of one or more monocyte subtypes. The defined set of one or more lymphocyte subtypes and the defined set of one or more monocyte subtypes may be different for different types of cancers.

In another example, the disclosure is directed to a method of identifying one or more favorable dates to deliver a pharmacological treatment to a patient, comprising: receiving data corresponding to levels of one or more lymphocyte subtypes in blood samples from the patient over an observed time period; receiving data corresponding to levels of one or more monocyte subtypes in blood samples from the patient over the observed time period; for each of the lymphocyte subtypes, fitting a periodic function to the received data corresponding to the levels of the one or more lymphocyte subtypes in the blood samples of the patient, and extrapolating the fitted periodic function to a plurality of proposed treatment dates occurring subsequent to the observed time period; for each of the monocyte subtypes, fitting a periodic function to the received data corresponding to the levels of the one or more monocyte subtypes in the blood samples of the patient, and extrapolating the fitted periodic function to the plurality of proposed treatment dates occurring subsequent to the observed time period; for each of the lymphocyte subtypes, determining a state of the extrapolated fitted periodic function on each of the plurality of proposed treatment dates, wherein the state on a proposed treatment date is determined to be favorable if the extrapolated fitted periodic function is greater than a threshold value associated with the lymphocyte subtype on the proposed treatment date; for each of the monocyte subtypes, determining a state of the extrapolated fitted periodic function on each of the plurality of proposed treatment dates, wherein the state on a proposed treatment date is determined to be favorable if the extrapolated fitted periodic function is less than a threshold value associated with the monocyte subtype on the proposed treatment date; and identifying at least one of the plurality of proposed treatment dates as a favorable date to deliver the pharmacological treatment to the patient, wherein the identified favorable date to deliver the pharmacological treatment to the patient corresponds to the proposed treatment date on which a maximum number of the lymphocyte subtypes are determined to be in a favorable state and a maximum number of the monocyte subtypes are determined to be in a favorable state.

The method may further include developing a treatment plan for the patient based on the at least one favorable date to deliver the pharmacological treatment to the patient. The method may further include delivering the pharmacological treatment to the patient on the at least one favorable date to deliver the pharmacological treatment to the patient. The fitted periodic functions are sinusoidal periodic functions.

In another example, the disclosure is directed to a method of identifying one or more favorable dates to deliver a pharmacological treatment to a patient, comprising: receiving data corresponding to concentrations of one or more biological variables in blood samples from the patient over an observed time period; for each of the biological variables, fitting a periodic function to the received data corresponding to the concentration of the biological variable in the blood samples of the patient; for each of the biological variables, extrapolating the corresponding fitted periodic function to a plurality of proposed treatment dates occurring subsequent to the observed time period; for each of a first set of the one or more biological variables and on each of the plurality of proposed treatment dates, determining a state of the biological variable on the proposed treatment date, wherein the state is determined to be favorable if the corresponding fitted periodic function is greater than a corresponding threshold value; for each of a second set of the one or more biological variables and on each of the plurality of proposed treatment dates, determining a state of the biological variable on the proposed treatment date, wherein the state is determined to be favorable if the corresponding fitted periodic function is less than a corresponding threshold value; and identifying at least one of the plurality of proposed treatment dates as a favorable date to deliver the pharmacological treatment to the patient based on the determined state for each of the biological variables.

The method may further include, for each of the biological variables, determining a threshold value based on the received data corresponding to the concentration of the biological variable in the blood samples of the patient, wherein the state of the biological variable is determined to be UP if the fitted periodic function is greater than the threshold value on a proposed treatment date, and wherein the state of the biological variable is determined to be DOWN if the fitted periodic function is less than the threshold value on a proposed treatment date. The method may further include, wherein if the biological variable is a lymphocyte subtype, the state of the biological variable is favorable if the state is determined to be UP on the proposed treatment date, and wherein if the biological variable is a monocyte subtype, the state of the biological variable is favorable if the state is determined to be DOWN on the proposed treatment date.

In another example, the disclosure is directed to a method of cancer treatment, comprising administering chemotherapy treatment to a patient on a favorable treatment date identified based on a predicted lymphocyte-to-monocyte ratio in the blood of the patient on the favorable treatment date.

In another example, the disclosure is directed to a method of cancer treatment, comprising administering chemotherapy treatment to a patient on a favorable treatment date identified based on a predicted state of at least one lymphocyte subtype in the blood of the patient on the favorable treatment date, and on a predicted state of at least one monocyte subtype in the blood of the patient on the favorable treatment date.

The method may further include wherein the predicted state of the at least one lymphocyte subtype is favorable if a value of a periodic function fitted to concentration values of the at least one lymphocyte subtype in the blood of the patient over an observed period of time and extrapolated to a proposed treatment date is greater than a first threshold value on the proposed treatment date, and wherein the predicted state of the at least one monocyte subtype is favorable if a value of a periodic function fitted to concentration values of the at least one monocyte subtype in the blood of the patient over an observed period of time and extrapolated to a proposed treatment date is less than a second threshold value on the proposed treatment date.

The details of one or more examples are set forth in the accompanying drawings and the description below. Other features and/or advantages will be apparent from the description and drawings, and from the claims.

### BRIEF DESCRIPTION OF DRAWINGS

FIGS. 1A-1C are flowcharts illustrating an example overall process for determination of time(s) for delivery of chemotherapy treatment.

FIGS. 2A and 2B show the frequency of 9 example functions as concentration dynamics of 28 cytokines and 25 cell subtypes for 10 patients.

FIG. 3 shows the sum of ranks for each of the 10 patients compared with the clinical outcome for each individual patient.

FIG. 4 shows extrapolated relative CRP concentration (right axis, dashed bars) and relative first derivative of the fitted function on the day of treatment (left axis, black bars) as related to PFS of the patients.

FIG. 5 shows the relationship between progression free survival (PFS) time (days) and sum of ranks of IL-12p70 and CD197/CD206 ratio.

FIGS. 6 and 7 show nonlinear regression fitting of CD197/CD206 ratio time dependent fluctuations in patients #1 (PFS=916 days) and patient #2 (PFS=37 days).

FIGS. 8A-8C show synthetic virtual concentration/cell count curves showing dynamic of one variable in several patients.

FIGS. 9A and 9B show relative concentration (right axis, dashed bars) and relative first derivative of the fitted function on the day of treatment (left axis, black bars) as related to PFS of the patients.

FIG. 10 is a block diagram illustrating an example system for determination of time(s) for delivery of chemotherapy treatment.

FIG. 11 illustrates an example simulation which considered three different observation periods (10, 15 and 20 days), three various sampling frequency (every day, every other day and 1-2 days), one hundred amplitudes and twenty periods

FIGS. 12A-12C are graphs illustrating example frequency distribution of R² for various ranges and datasets.

FIGS. 13A-13C are graphs illustrating example frequency distribution of R² for an example 5-2-5 sample collection schedule.

FIG. 14 is a graph illustrating example frequency distribution of R² for an example 5-2-5 sample collection schedule.

FIG. 15 is a chart illustrating an example association between the 5-day period of actual chemotherapy application, time predicted by the example clustering algorithm and PFS in 8 melanoma patients.

FIGS. 16A-16C are example graphs illustrating counts of variables profiles for IL-12p70 (FIG. 16A), IL-17 (FIG. 16B) and CRP (FIG. 16C).

FIGS. 17A and 17B are example graphs illustrating example clustering of concentration profiles IL-1ra and IL-12p70 in Patient #1 (PFS=916 days) (FIG. 17A) and concentration profiles IL-1ra and IL-12p70 in Patient #2 (PFS=37 days) (FIG. 17B).

FIG. 18 is a flowchart illustrating another example process by which a controller may determine favorable treatment time(s) for delivery of chemotherapy treatment (or other type of pharmacological treatment) to a patient.

FIG. 19 is a graph illustrating the states of selected biological variables vs. progression-free survival (PFS) on the day of therapy administration for 14 patients in a clinical trial.

FIG. 20 is a block diagram illustrating another example system for determination of one or more favorable dates for delivery of pharmacological or other therapeutic treatment.

FIG. 21 is a flowchart illustrating another example process by which a controller may determine favorable treatment date(s) for delivery of chemotherapy treatment (or other type of therapeutic or pharmacological treatment) to a patient.

FIG. 22 is a graph illustrating example lymphocyte and monocyte oscillations and identification of a favorable date, Rₓ, for delivery of therapeutic treatment to a patient based on a prognostic value of lymphocyte/monocyte ratio.

FIG. 23 is a graph illustrating example lymphocyte (square-shaped data points) and monocyte (diamond-shaped data points) oscillations and identification of a favorable date, Rₓ, for delivery of therapeutic treatment to a patient.

FIG. 24 is a chart illustrating PFS and the state (UP or DOWN) for CDC11c.14 monocytes and CD3.4 lymphocytes.

FIG. 25 shows the relative difference of concentration/counts (up—black or down—white) of 5 immune parameters (VEGF, Treg cells, CD11c.14, CD3.8 and CD3.4 cells) before and after timed delivery of therapeutic treatment as described herein as related to disease progression (PFS in days).

FIGS. 26A and 26B show blood concentration range of CD3+4+ cells (FIG. 26A) and CD11c+14+ cells (FIG. 26B).

FIG. 27 shows mean concentration of CD3+4+ (triangles, solid line) and CD11c+14+ (squares, dashed line) cells for each patient sorted in ascending order of CD3+4+ concentration.

FIG. 28 shows the dynamic of lymphocyte-to-monocyte ratio in melanoma patients.

FIG. 29 shows receiver operating characteristic curve for lymphocyte-to-monocyte ratio (LMR) on the day of initiation of treatment with temozolomide.

FIG. 30 shows the association of PFS with LMR state (LMR>1 or LMR<1) is represented as a heat map.

FIGS. 31A and 31B show a distribution of FC values in melanoma patients before (CY1) and after (CY2) treatment.

FIG. 32 is a flowchart illustrating another example process by which a controller may determine favorable treatment date(s) for delivery of chemotherapy treatment (or other type of therapeutic or pharmacological treatment) to a patient.

### DETAILED DESCRIPTION

In general, the disclosure relates to planning delivery of chemotherapy or other pharmaceutical treatment. For example, systems and/or methods described herein analyze time-dependent fluctuations of at least one biological variable measured in blood samples of a patient and determine one or more favorable dates for delivery of pharmaceutical treatment to the patient. In some examples, the biological variables are immune variables.

The measurements of the one or more biological variables may be indicative of the level of systemic inflammation in cancer patients. In the examples described herein, the techniques are described with respect to patients with metastatic melanoma. However, the techniques may also be applied to patients with other types of cancer.

In some examples, to identify which of the biological variables are indicative of favorable time(s) to deliver treatment to these patients, the systems and/or methods ascertain whether or not one or more biological variables are stable or variable over time, and if variable, in what systemic immune context. That is, curve-fitting is applied to time series data for each patient to determine the best fit variable function for each of the measured biological variables.

Once the best fit variable function is established, the treatment planning techniques described herein therapeutically utilize the variation of one or more biological variables over time information and devise a treatment strategy which, by using timed administration of conventional cytotoxic therapy (chemotherapy), may augment anti-tumor immunity and affect clinical outcomes.

In an example clinical trial described herein, the patient population included patients with unresectable stage IV malignant melanoma. Eligible patients had unresectable, histologically confirmed stage IV disease, age over 18 years, measurable disease as defined by the Response Evaluation Criteria in Solid Tumors (RECIST), Eastern Cooperative Oncology Group (ECOG) performance status (PS) of 0-2, and life expectancy ≥3 months. Both newly diagnosed, previously untreated patients, as well as patients who have had prior therapy for their metastatic disease were enrolled.

Treatment was initiated with temozolomide (TMZ) 150 mg/m² on days 1-5 on cycle 1 and the dose was increased to 200 mg/m² for all subsequent cycles if tolerated. Patients were treated every 4 weeks until progression, unacceptable toxicity or patient refusal. Prior to initiation of first chemotherapy cycle, eligible patients underwent peripheral blood testing for immunological biomarkers (immune variables) every 2-3 days for a period of two weeks. The blood samples were tested for a total of 52 variables; that is, 52 measurements of cytokine concentrations and cell counts in blood samples. The 52 variables are listed in Table 1.

TABLE 1

| | Variable |
|---|---|
| 1 | IL-10 |
| 2 | IL-12p70 |
| 3 | G-CSF |
| 4 | IL-9 |
| 5 | VEGF |
| 6 | CD206 |
| 7 | IL-1ra |
| 8 | IL-13 |
| 9 | CD4/294 |
| 10 | CD11c/14 |
| 11 | CD197/CD206 |
| 12 | DR(hi) |
| 13 | IL-15 |
| 14 | IL-17 |
| 15 | IL-6 |
| 16 | IL-8 |
| 17 | Eotaxin |
| 18 | TGF-b (ng/ml) |
| 19 | CD11c/CD123 |
| 20 | Treg (% gated) |
| 21 | IL-4 |
| 22 | IL-5 |
| 23 | GM-CSF |
| 24 | MIP-1a |
| 25 | MIP-1b |
| 26 | CD3−/16+56 |
| 27 | CD3−/CD16− |
| 28 | TIM3:CD294 |
| 29 | DR/11c (DC1) |
| 30 | DR/123 (DC2) |
| 31 | B7-H1 (DRhi) |
| 32 | IL-7 |
| 33 | FGF |
| 34 | IFN-g |
| 35 | IP-10 |
| 36 | CD3/4 |
| 37 | CD3/8 |
| 38 | CD4/TIM3 |
| 39 | B7-H1 (DRlo) |
| 40 | Treg (% total) |
| 41 | CRP pmol/L |
| 42 | IL-1b |
| 43 | IL-2 |
| 44 | RANTES |
| 45 | TNF-a |
| 46 | CD3/62L |
| 47 | CD197 |
| 48 | MCP-1 |
| 49 | PDGF |
| 50 | CD3 |
| 51 | DR (lo) |
| 52 | CD3/69 |

The time series of six CRP concentration measurements was fitted to a sine curve. The curve was then extrapolated for two periods and the next consecutive peaks of CRP concentration were predicted. Based on the periodicity of CRP oscillations, TMZ chemotherapy was initiated prior to the estimated time of the next CPR peak, or on day 14 post-registration if the peak could not be identified.

Peripheral blood samples were obtained at baseline and every 2-3 days thereafter for 15 days prior to the first cycle of TMZ chemotherapy. In order to study the global behavior of the anti-tumor immune response, the samples were further analyzed for plasma concentration of 29 different cytokines/chemokines/growth factors and the percentage of 22 immune cell subsets. All biospecimens were collected, processed, and stored in uniform fashion following established standard operating procedures. To reduce inter-assay variability, all assays were batch-analyzed after study completion.

The data was obtained as follows. However, it shall be understood that the data could be obtained in other ways, and that the disclosure is not limited in this respect. Peripheral blood mononuclear cell (PBMC) immunophenotyping for immune cell subset analysis. Blood was separated into plasma and PBMC using a density gradient (Ficol-hypaque, Amersham, Uppsala, Sweden). Plasma samples were stored at −70° C., and PBMC were stored in liquid nitrogen. PBMC bio-specimens were analyzed for the frequencies of T cells (CD3+), T helper cells (CD3+4+), CTL (CD3+8+), natural killer cells (NK, CD16+56+), T helper 1 (Th1) cells (CD4+TIM3+), Th2 cells (CD4+294+), T regulatory cells (Treg, CD4+25+FoxP3+), type 1 dendritic cells (DC1, CD11c+HLA-DR+), DC2 (CD123+HLA-DR+), type 1 macrophages (M1, CD14+197+), type 2 macrophages (M2, CD14+206+) and for the activation status of these cell types. Immunophenotyping of PBMC was performed by flow cytometry using FITC- and PE-conjugated antibodies to CD3, CD4, CD8, CD16, CD56, CD62L, CD69, TIM3, CD294, HLA-DR, CD11c, CD123, CD14, CD197, CD206, and B7-H1 (Becton-Dickinson, Franklin Lakes, N.J.). In addition, intracellular staining for FoxP3 (BioLegend, San Diego, Calif.) was performed according to the manufacturer's published instructions. Data were processed using Cellquest® software (Becton-Dickinson, Franklin Lakes, N.J.). In order to access the Th1/Th2 balance PBMC were stained with anti-human CD4, CD294, and TIM-3. The stained cells were analyzed on the LSRII (Becton Dickinson Franklin Lakes, N.J.). The CD4 positive population was gated and the percent of CD4 cells positive for either CD294 or TIM-3 was determined. Preliminary data suggests that CD4/CD294 positive Th2 cells exclusively produce IL-4 and not IFN-γ upon PMA and ionomycin stimulation. Conversely, CD4/TIM-3 positive Th1 cells exclusively produce IFN-γ and not IL-4 following the same in vitro stimulation. Enumeration of Treg was performed using intracellular staining for FoxP3 of CD4/25 positive lymphocytes.

Protein levels for 29 cytokines, chemokines, and growth factors, including IL-1β, IL-1rα, IL-2, IL-4, IL-5, IL-6, IL-7, IL-8, IL-9, IL-10, IL-12(p70), IL-13, IL-15, IL-17, basic fibroblast growth factor (FGF), Eotaxin, granulocyte colony-stimulating factor (G-CSF), granulocyte-macrophage colony-stimulating factor (GM-CSF), interferon γ □IFN-γ), 10 kDa interferon-gamma-induced protein (IP-10), macrophage chemoattractant protein 1 (MCP-1), migration inhibitory protein 1α (MIP-1α), MIP-1β, □platelet-derived growth factor (PDGF), Regulated upon Activation Normal T-cell Expressed and Secreted (RANTES), tumor necrosis factor α (TNF-α), vascular endothelial growth factor (VEGF), CRP, and transforming growth factor beta (TGF-β1) were measured using the BioRad human 27-plex cytokine panel (Cat #171-A11127, Bio-Rad, San Diego Calif.) as per the manufacturer's instructions. Plasma levels of TGF-β1 were determined using the duoset capture and detection antibodies (R and D Systems Minneapolis, Minn.) as per manufacturer's instructions. Briefly, plasma samples were treated with 2.5 N Acetic acid and 10M urea to activate latent TGF-β1 followed by neutralization with NaOH and HEPES. The activated samples were added to plates, which had been coated with a mouse anti-human TGF-β1. After incubation the wells were washed and biotinylated chicken anti-human TGF-β1 detection antibody was added. The color was developed using streptavidin-HRP and R and D systems substrate kit. Plasma levels of TGF-β1 were calculated using a standard curve from 0-2000 pg/ml.

All plasma cytokine measurements were performed in duplicate. Normal values for plasma cytokine concentrations were generated by analyzing 30 plasma samples from healthy donors (blood donors at the Mayo Clinic Dept. of Transfusion Medicine). A set of three normal plasma samples (standards) were run along side all batches of plasma analysis in this study. If the cytokine concentrations of the “standard” samples differed by more than 20%, results were rejected and the plasma samples re-analyzed.

The data for each of the variables was then applied to a curve fitting process to determine whether each cyctokine concentration/cell count followed a predictable variation over time. For example, the data for each variable was applied to each of the functions shown in Table 2:

TABLE 2

| | |
|---|---|
| F1 | Linear function y = ax + b |
| F2 | Exponential Fit: y = ae\hat{ }(bx) |
| F3 | Exponential Association: y = a(1 − exp(−bx) |
| F4 | Logistic Model: y = a/(1 + b * exp(−cx)) |
| F5 | Quadratic Fit: y = a + bx + cx\hat{ }2 |
| F6 | Sinusoidal Fit: y = a + b * cos(cx + d) |
| F7 | Rational Function: y = (a + bx)/(1 + cx + dx\hat{ }2) |
| F8 | Gaussian Model: y = a * exp((−(b − x)\hat{ }2)/(2 * c\hat{ }2)) |
| F9 | MMF Model: y = (a * b + c * x\hat{ }d)/(b + x\hat{ }d) |
| F0 | No Fit |
| F0 | No DATA |

In the example described herein, CurveExpert 1.4 software (Daniel G. Hyams Hixson, Tenn.) and GraphPad Prizm 4.0 software (GraphPad Software Inc. La Jolla Calif.) were used to construct time-dependent profiles of plasma cytokine concentrations and immune cell counts by fitting data points to the selected mathematical functions. Both software packages use Levenberg-Marquart (LM) algorithm to solve nonlinear regressions to fit experimental data to a model curve. The correlation coefficient r=√(Sₜ−Sᵣ)/Sₜ calculated by CurveExpert may be used as the first criterion for goodness of fit, where Sₜ considers the distribution around a constant line and is calculated as Sₜ=Σ(y−yᵢ)² and Sᵣ considers the deviation from the fitting curve and is calculated as Sᵣ=Σ(yᵢ−f(xᵢ))². GraphPad Prizm was used to obtain R² values, 95% confidence intervals for the variables of the fitted functions, and 95% confidence bands for the fitted curves. R² is calculated as R²=1−Sᵣ/Sₜ. These parameters may be used as selection criteria in different steps of the analysis as described below.

Although specific commercially available software packages are described herein to perform the curve fitting analysis, it shall be understood that other software packages or custom software could also be used to perform the curve fitting analysis, and that the disclosure is not limited in this respect. In addition, mathematical methods other than an Levenberg-Marquart (LM) analysis, such as Fourier transform, autocorrelation methods, or other mathematical of determining or identifying a periodic pattern in a data set, may be used can be used to reveal periodical pattern of concentration and cell count fluctuation and define the function.

The purpose of the curve fitting analysis is to determine whether any of the measured immune variables change in a predictable fashion following a cyclical pattern (dynamic equilibrium of immunity and cancer). Therefore, the goal of the curve fitting analysis is to assess whether concentrations of plasma cytokines/chemokines and immune cells fluctuate, and if so, to determine whether these fluctuations follow a mathematically predictable cyclical pattern. To that end, the plasma levels for the 52 immune variables (29 different cytokines/chemokines/growth factors and the percentage of 22 immune cell subsets) in serial blood samples collected every 2-3 days prior to initiation of TMZ therapy were measured in 10 patients with metastatic malignant melanoma. Of the 12 enrolled patients, number of data points was inadequate for curve-fitting analysis in two patients; one patient was hospitalized shortly after enrollment, and the other had an insufficient number of successive blood samples obtained prior to initiation of TMZ therapy. Technical reproducibility was assessed by the coefficient of variation among duplicates (average coefficient of variation was 5.13% for 1593 data points).

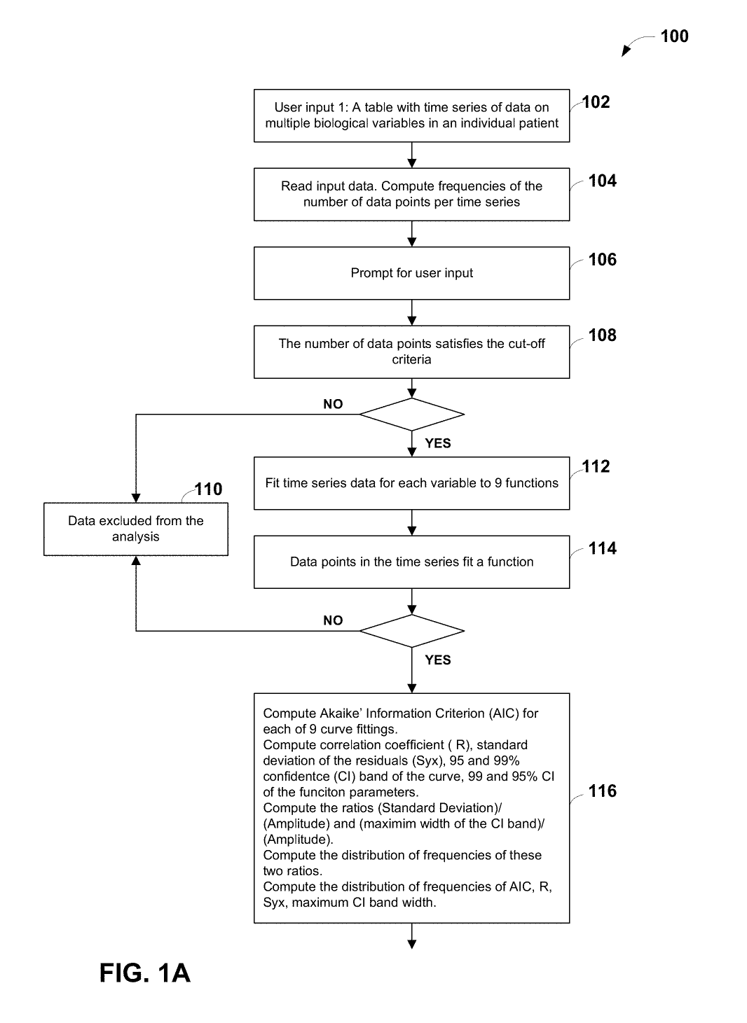

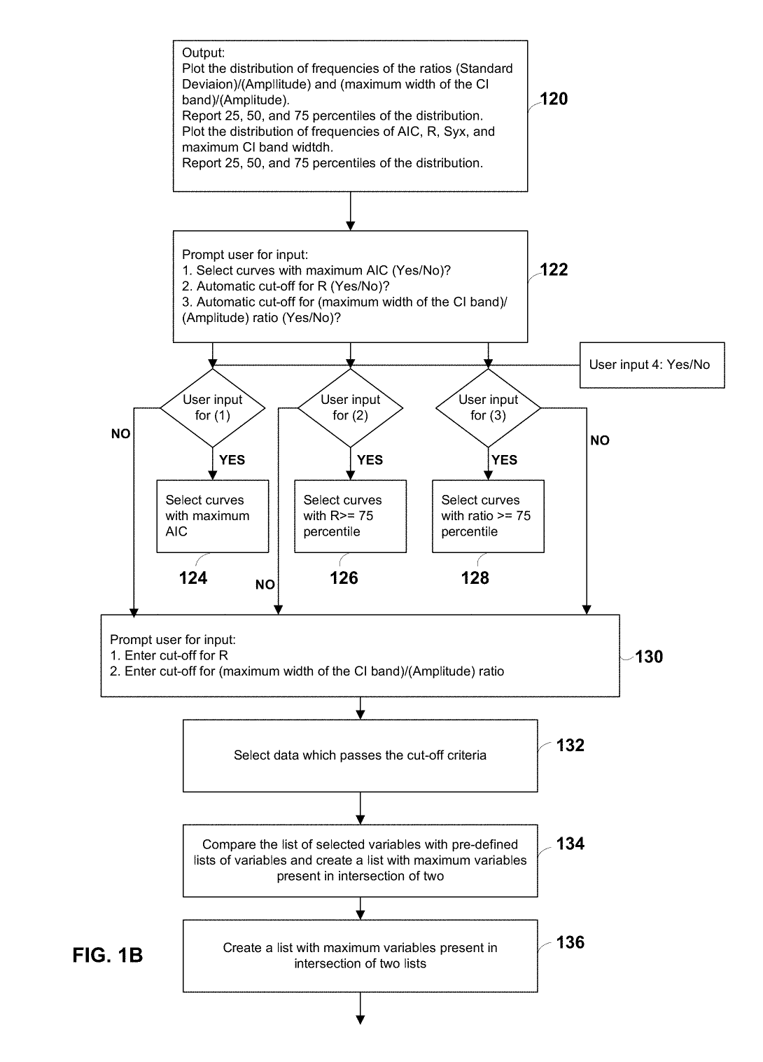

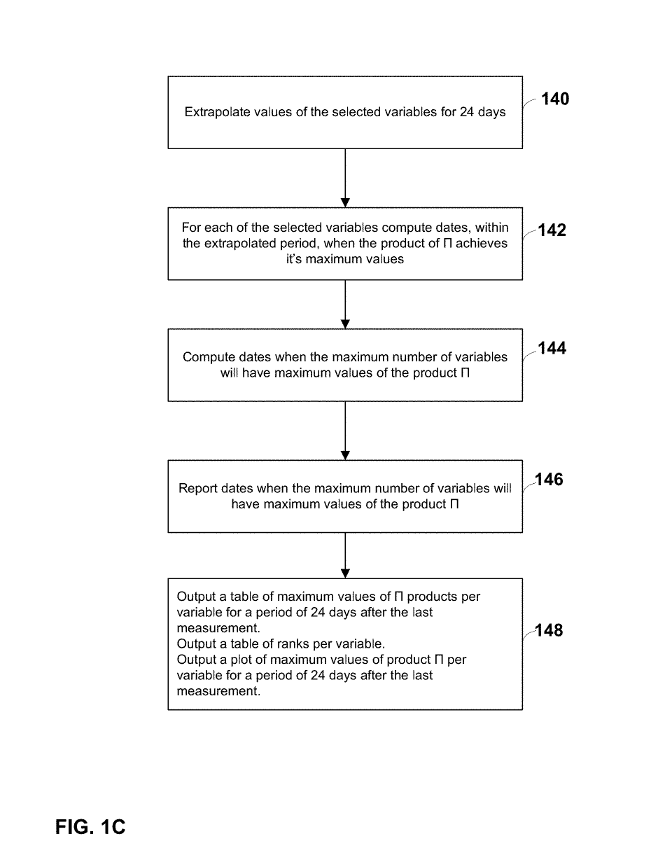

FIGS. 1A-1C are flowcharts illustrating an example overall process 100 for determination of favorable times for delivery of chemotherapy or other pharmacological treatment. For purposes of the present description, cytokine concentration or cell counts will be denoted as “immune variables” and cytokine concentration or cell count measured in an individual patient on a specific day as a data point. Time-dependent profiles for each variable and each patient were constructed by fitting the data points to each of 10 possible functions (e.g., the 9 mathematical functions plus “no fit” function listed in Table 2).

FIG. 1A shows the process by which presence of a regular pattern in fluctuation of cytokines' concentration and cell counts is determined. FIG. 1B shows the process of determining the correlation between clinical outcome and the presence of a pattern in the variance of the immune variables. FIG. 1C shows an example process by which a proposed time of therapy for a particular patient may be determined based on the curve fitting(s) for one or more selected immune variables.

The curve fitting analysis was performed based on 6 or 7 sequential measurements (time points) for each variable/patient over a period of 15-days. The “goodness of fit” of the measured variables with a mathematically predicted function was estimated statistically using the correlation coefficient calculated by CurveExpert 1.4 software (REF/source). The cut-off criteria for good fit were computed as follows: (a) the frequency distribution of the correlation coefficient was computed across all profiles and all patients; and (b) the value of the 75^{th} percentile (0.86) was accepted as a cut-off to eliminate profiles which did not fit a model well.

As shown in FIG. 1A, the process receives time series of data on one or more biological immune variables in an individual patient (102) and a date of treatment start. To ensure that each time series includes sufficient data to perform each curve fitting, the process computes the frequencies of the number of data points per time series (104). If the number of data points does not satisfy a user input cut-off criteria, the data may be excluded from the analysis.

If the number of data points satisfies the user input cut-off criteria (106, 108), the process fits the time series data for each immune variable to each of a set of mathematical functions (112). In this example, the process fits the time series data to each of the 9 functions listed in Table 2. However, it shall be understood that more or fewer functions may be used, and that other functions not listed in Table 2 may also be used, and that the disclosure is not limited in this respect.

If the data points fit a function (114), the process may compute various parameters indicative of the “goodness” of the fit of the time series data to each of the functions (116). For example, the process may compute Akaike's Information Criterion (AIC) for each of 9 curve fittings; compute a correlation coefficient (R), a standard deviation of the residuals (S_{yx}), 95 and 99% confidence (CI) band of the curve, 99 and 95% CI of the function parameters; compute the ratios (Standard Deviation)/(Amplitude) and (maximum width of the CI band)/(Amplitude); compute the distribution of frequencies of these two ratios; and/or compute the distribution of frequencies of AIC, R, S_{yx}, maximum CI band width.

As shown in FIG. 1B, the process may next report and/or plot the distribution of frequencies of the ratios (Standard Deviation)/(Amplitude) and (maximum width of the CI band)/(Amplitude); report 25, 50 and 75 percentiles of the distribution; plot the distribution of frequencies of AIC, R, S_{yx}, and maximum CI band width; report 25, 50 and 75 percentiles of the distribution (120). It shall be understood that more or fewer of these parameters may be computed and/or plotted, and that other parameters not specifically shown herein may be determined, and that the disclosure is not limited in this respect.

The process may next prompt user for input (122). For example, the process may prompt the user to input one or more of the following: 1. Select curves with maximum AIC (Yes/No)? (124); 2. Automatic cut-off for R (Yes/No)? (126); 3. Automatic cut-off for (maximum width of the CI band)/(Amplitude) ratio (Yes/No)? (128).

If the user does not enter automatic cut-offs, the process may prompt user for input 913). For example, the process may prompt the user to: 1. Enter cut-off for R; and/or 2. Enter cut-off for (maximum width of the CI band)/(Amplitude) ratio.

The process may then select the immune variables corresponding to the data series which pass the cut-off criteria (132). The process may then compare the list of selected immune variables with lists of pre-defined variables (determined by, for example, the ranked list of immune variables) (134). The process may then find an intersection set of the two lists which contains the maximum number of immune variables (136). The process may then create a list of these immune variables and continue the analysis with this list.

The resulting list contains those immune variables having the highest correlation with PFS for that particular patient.

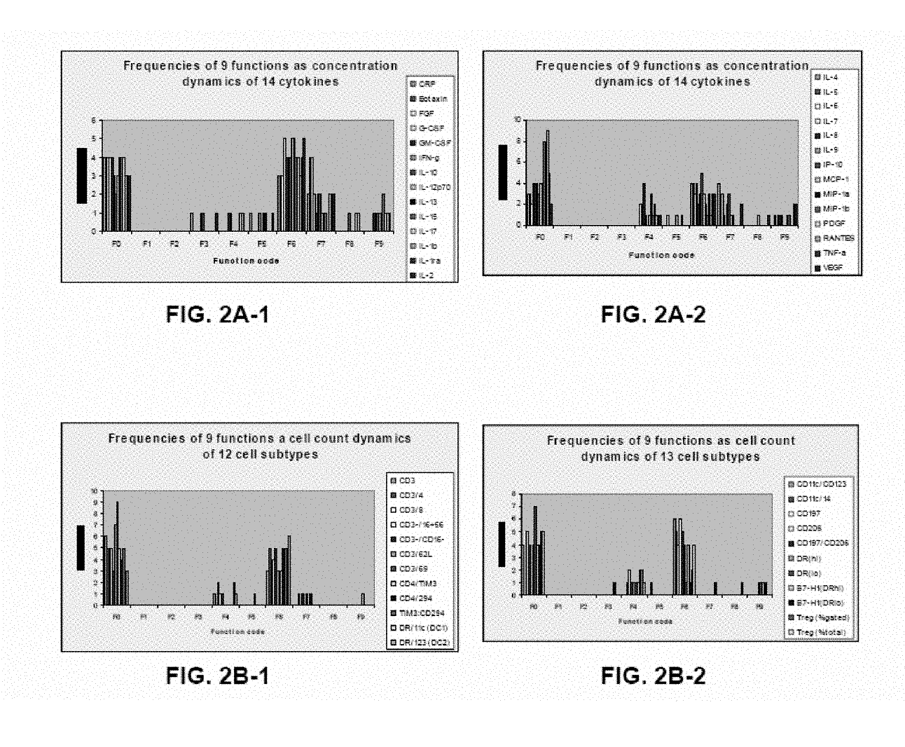

FIGS. 2A-1 and 2A-2 show the frequency of the 9 example functions as concentration dynamics of 14 cytokines and 14 cytokines, respectively, for 10 patients. FIGS. 2B-1 and 2B-2 show the frequency of the 9 functions as cell count dynamics of 12 cell subtypes and 13 cell subtypes, respectively, for 10 patients. The cytokine legend and color code is described on the right-side of figure. Function codes: F1=Linear function y=ax+b; F2=Exponential Fit: y=aê(bx); F3=Exponential Association: y=a(1−exp(−bx); F4=Logistic Model: y=a/(1+b*exp(−cx)); F5=Quadratic Fit: y=a+bx+cx̂2; F6=Sinusoidal Fit: y=a+b*cos(cx+d); F7=Rational Function: y=(a+bx)/(1+cx+dx̂2); F9=Gaussian Model: y=a*exp((−(b−x)̂2)/(2*ĉ2)); F10=MMF Model: y=(a*b+c*x̂d)/(b+x̂d); F0=No Fit/No data.

The example distributions shown in FIGS. 2A and 2B frequencies of all 9 mathematical models (functions) shows that most time-dependent profiles fit sinusoidal or rational functions.

In order to establish whether an ordered pattern of fluctuation correlates with clinical outcome (progression free survival or PFS), an index of fitness is assigned to each variable, patients are ranked by the sum of indices, and the correlation coefficient between this rank and the PFS is calculated. In one example, the assigned index was 1 if the profile fitted a function well (correlation coefficient≥0.86) and the function was biologically possible. Functions with infinite growth or infinite decline were considered biologically implausible as their extrapolation produces biologically impossible values (e.g. <0) for plasma cytokine concentrations or cell count frequencies and were assigned an index of zero (0). The index was −1 if a profile did not fit any function. Using these criteria, the sum of these indices was then calculated for each immune variable per individual patient.

For example, if IL-10 concentration dynamically fitted to cosine, rational or logistic functions in 7 patients and fitted an exponential growth (biologically impossible) function in one patient, this would produce a score of 7 (7×1+0=7). Table 3 shows the rank for each of the 52 immune variables in the example clinical trial.

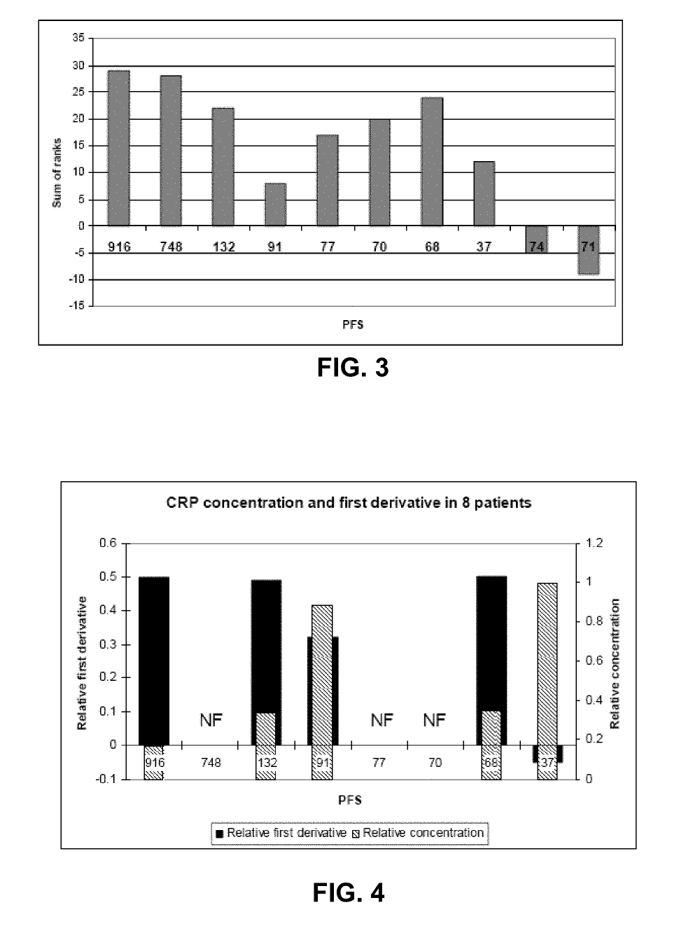

FIG. 3 shows the sum of ranks for each of the 10 patients compared with the clinical outcome for each individual patient. The data suggests that the patients with the highest rank (fluctuation of cytokine concentrations and/or cell counts follows an ordered pattern) experienced the best clinical outcomes (PFS of 916 and days for ranks 29 and 28, respectively). Surprisingly, the subjects with the lowest (−5 and −9, respectively) rank score (entirely random fluctuation of cytokine concentrations/cell counts) identified by this method were the two patients with metastatic ocular melanoma. These two patients were not studied further given the inability to fit them to any mathematical model.

Separate analysis of the remaining eight patients with metastatic cutaneous melanoma resulted in a correlation coefficient between the total individual score and PFS of 0.72. In a similar way scores (sum or indices) were assigned to each variable. In this case indices were summed across patients per individual variable. Table 3 shows the resulting rank for each of the 52 example immune variables.

TABLE 3

| Rank | Variable |
|---|---|
| 7 | IL-10, IL-12p (70), G-CSF |
| 6 | IL-9, VEGF, CD206 |
| 5 | IL-1rα, IL-13, IL-15, IL-17, CD4/294, CD11c/14, CD197/CD206, |
| | DR (hi) |
| 4 | IL-6, IL-8, Eotaxin, TGF-b, Treg (% gated) CD11c/CD123 |
| 3 | IL-4, IL-5, GM-CSF, MIP-1a, MIP-1b, CD3−/16+56+, |
| | CD3−/CD16−, DR/11c (D1), DR/123 (D2), TIM3: CD294, B7-H1 |
| | (DRhi) |
| 2 | IL-7, FGF, IFN-g, IP-10, CD3/4, CD3/8, CD4/TIM3, B7-H1 |
| | (DRlo), Treg (% total) |
| 1 | CRP, IL-1b, IL-2, RANTES, TNF-α, CD3/62L, CD197 |
| 0 | MCP-1, PDGF, CD3, DR (lo) |
| −1 | CD3/69 |

Determining which immune variables correlate with clinical outcome. In order to understand if certain of the measured immune variables of immune function had a greater/lesser impact on survival, as measured by cyclical function, additional analyses were performed on the 14 variables assigned a score of 5 or greater in the 8 patients with metastatic cutaneous melanoma (see, e.g., Table 3).

As described above, the index assigned to each variable was 1 if the profile fits a function, 0 for time dependent profiles of variables which fitted biologically impossible functions, and −1 if a profile did not fit any function. As the maximum theoretical score of an immune variable was 8 in this example (8 patients), the cut-off of 5 was chosen because it eliminated those variables which fit a function in <50% of patients. In the case of larger trials (more patients) the cutoff could be chosen appropriately. The maximum score obtained for the remaining variables was 7. These included IL-1rα, IL-9, IL-10, IL-12(p70), IL-13, IL-15, IL-17, G-CSF, VEGF, Th2 T-helper lymphocyte subset (CD4/294), CD11c-positive monocytes (CD11c/14), the ratio of polarized M1/M2 macrophages (DD197/CD206) and DR(hi).

FIG. 1C illustrates an example process by which further analysis was performed on eight patients on variables with the score 5 or greater. The plasma cytokine concentration or the cell count was extrapolated on the day of treatment for the 14 selected variables in the eight patients analyzed (140). The first derivative of the fitted function on the day of treatment was calculated. The first derivative shows whether the function at that point is increasing (positive value), decreasing (negative value) or is not changing (zero) and the magnitude of the first derivative reflects the magnitude of the trend.

The range of plasma cytokine concentrations/cell counts varied significantly across patients. In order to be able to compare these concentrations in different patients, the concentrations/cell counts may be convereted into relative values by using the formula:

relative conc (“conc”)=(Cₘₐₓ−Cₑₓ)/(Cₘₐₓ−Cₘᵢₙ), where
Cₘₐₓ is the maximum concentration within the observed time period,
Cₘᵢₙ is the minimum concentration within the same period, and
Cₑₓ is the extrapolated concentration on the day of treatment.

The same conversion was applied to first derivative values. In the cases when both maximum and minimum first derivative were negative the following formula may be applied:

relative derivative (“der”)=−1*(1−(Dₘₐₓ−Dₑₓ)/(Dₘₐₓ−Dₘᵢₙ)), where
Dₘₐₓ is the maximum derivative within the observed time period,
Dₘᵢₙ is the minimum derivative within the same period, and
Dₑₓ is the derivative of the function for the extrapolated point corresponding to the day of treatment in order to compensate for the subtraction of two negative numbers.

The initial hypothesis was that application of treatment near the CRP concentration peak may be therapeutically advantageous by predicting the correct time point in the cycle when chemotherapy will selectively deplete replicating Tregs and other immunosuppressive elements and “unblock” the anti-tumor immune response. However, final data analysis showed no correlation between PFS and CRP concentration or the first derivative of the fitted function (see, e.g., FIG. 4) (correlation coefficients −0.47 and −0.36 respectively).

In this example, a single parameter may be used to characterize both the magnitude of change and the trend of the fluctuation for a given biological variable. This parameter may then be used to find a relationship between the fluctuation of plasma cytokines/immune cellular elements and clinical outcome and guide personalized “timed” chemotherapy delivery. In some examples, this parameter (referred to as index Pi or Π) may be obtained by exponentiating the relative concentration and the first derivative and calculating their product with the formula:

Π=e^{der×T}×e^{conc}, where
e^{der} is the number e (2.7182818 . . . ) raised to the power of the relative derivative,
e^{conc} is the number e raised to the power of relative concentration, and
T is function period in days to correct for variable period length.

The index Pi, as a product based on both the relative concentration and the relative derivative, takes into consideration both the magnitude of the concentration and the dynamic trend of a given variable at a precise time point in the immune response cycle, hence describing the time-dependent fluctuation of a certain immune biomarker more accurately than the protein concentration or cell count alone.

In the above example, index Pi is a product of exponentiated values, therefore it is converted into a sum by the transformation: e^{der×T}×e^{conc}=e^{(der×T)+conc}. Generally, in these examples the parameters of interest are der and conc, and the exponent alone may be taken as follows:
Π=(der×T)+conc, where
der is the relative derivative,
conc is the relative concentration, and
T is function period in days to correct for variable period length.

In other examples, a parameter Π could be computed that does not include the period (T). For example, T could be left out of either of the above equations, or out of other appropriate equations. Other equations may also be used. In general, an equation may include the value and magnitude of the trend without zeroing the product (unless both values are actually zero—which makes a zero legitimate result). That is, an index Pi may be calculated by any formula or numerical transformation which produces a linear or non-linear dependency of the result on both arguments (relative derivative and relative concentration) with the limitation that the result is zero only when both arguments equal zero. If one of the arguments equals zero, the equation does not produce a zero result.

In another example, relative values of the concentration and derivative can be calculated from the maximum and minimum values of the curve, fitted to the experimental data points.

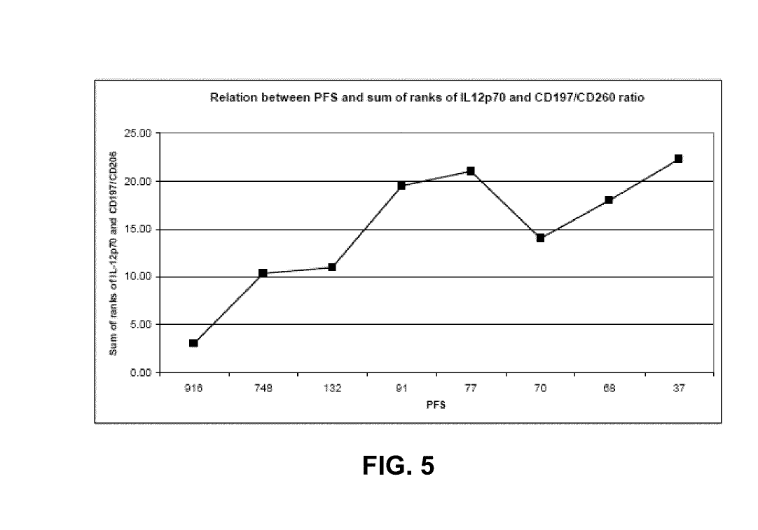

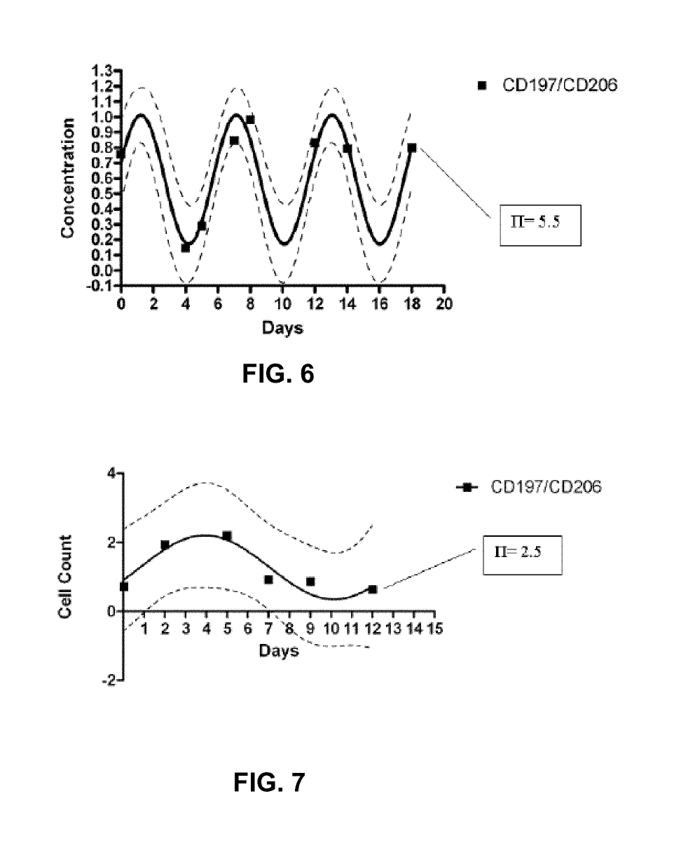

In terms of clinical application, the aim was to determine a relationship between concentration and dynamic trend of the variable at the day of treatment with clinical outcome. With this in mind, the goal was to find the variables with the highest correlation between the product Π on the day of treatment and PFS. In order to do that, the products Π were ranked in descending order for each measured immune variable. If an immune variable did not fit a biologically possible function, then the product could not be calculated and since 14 immune variables were analyzed and the lowest rank for a product was 14, it follows was the next lowest rank for a product which could not be calculated was 15. Because this rank is weighted by the proportion of non-fitted variables in a given patient, a weighted rank was used calculated as 15*(number of immune variables which do not fit a function)/(total number of measured variables). In this example, the correlation coefficient was used to assess the association between the rank of each of these 14 variables and the patients' PFS. In this example, two immune variables, the concentration of IL12p70 and the ratio of CD197/CD206 positive cells (ratio of polarized M1/M2 macrophages) had the highest correlation coefficients of −0.73 and −0.62, respectively. This was further supported by a correlation coefficient of −0.83 between the sum of the ranks for these two variables and PFS. Four patients (50%) with the sum of ranks of these two variables below 15 had average PFS of 466, whereas the other four with sum of ranks above 15 had average PFS of 68 (see, e.g., FIG. 5), suggesting that the value of the product Π on the day of treatment correlated favorably with clinical outcome. For instance, the product Π on the day of treatment for the patients at the two extremes were 5.5 in the patient with the highest PFS (916 days; corresponding rank=1) and 2.5 in the subject with the lowest PFS (37 days; corresponding rank=10) (see, e.g., FIGS. 6 and 7) Therefore, application of treatment at a time point when this product is elevated, meaning that the concentration is high and also on the rise, results in improved outcome.

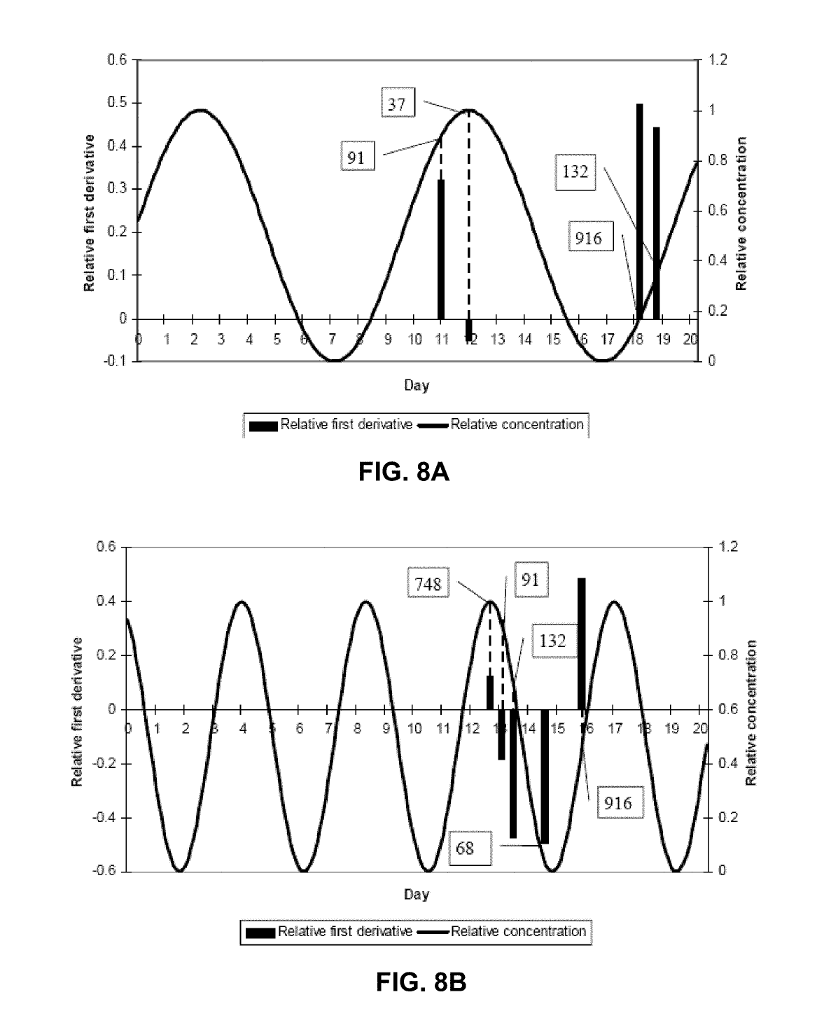

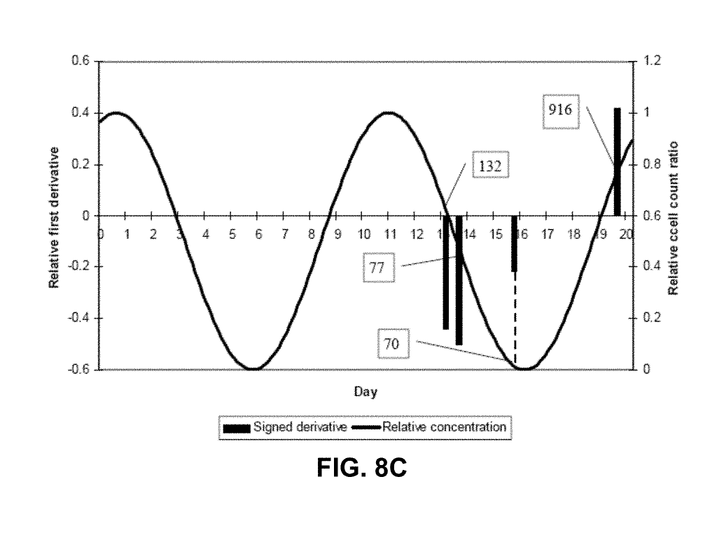

To better understand how the concentration of a cytokine or cell count and the trend for increase or decrease of these variables (first derivative of the fitted function) are related to the clinical outcome, the values of these variables in patients with different PFS were compared. A fitted cosine curve was computed where all four parameters of the cosine function (a, b, c and d) were average values of the corresponding parameter across patients being compared and a variable being analyzed. The resulting curve represented averaged concentration/cell count dynamics for several patients on a relative concentration scale (calculation of relative concentration is described above). First derivatives of the fitted function on the treatment day were also plotted on a relative scale (FIGS. 8A-8C). In effect, the plot shows relative concentration and relative first derivative on the treatment day for several patients with different PFS. Concentration/cell count and first derivative plots were constructed for CRP, IL-12p70 and CD197/CD206 for patients in whom these variables fitted a cosine function. These figures demonstrate, that the clinical outcome (PFS) directly correlated with concentration or first derivative for the given measurements (FIGS. 8A-8C).

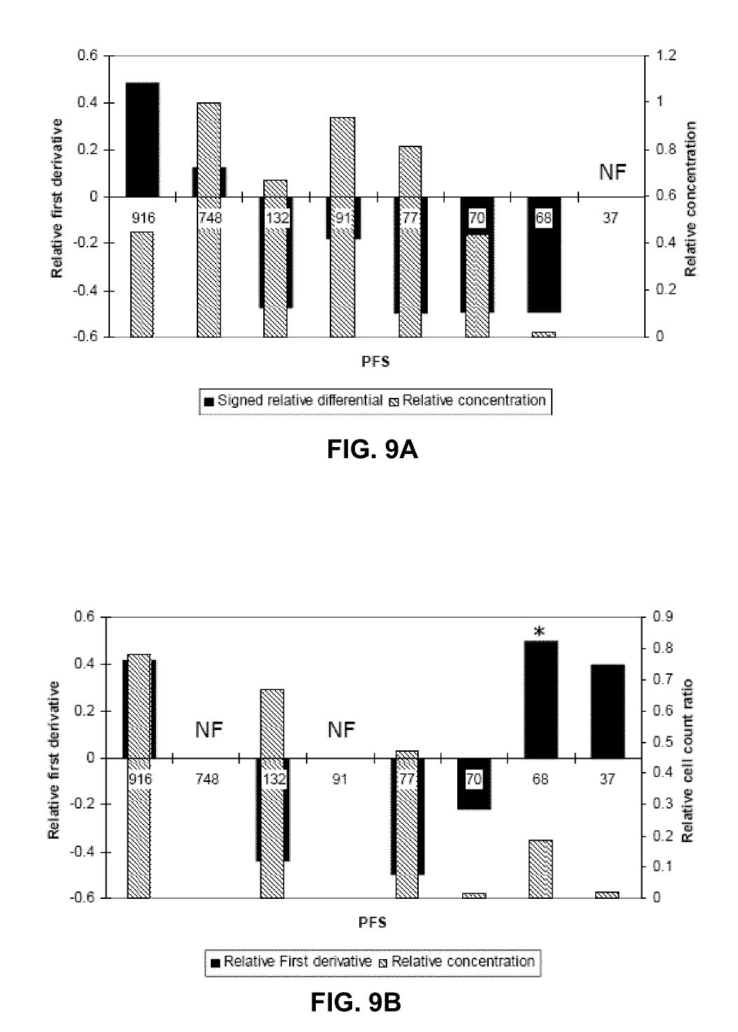

In attempt to further generalize this observation, concentration/cell count ratio and first derivative of on the day of treatment across 8 patients for IL-12p70, CRP and CD197/CD206 were compared. The values were compared as relative values for a given variable in each patient. FIGS. 9A and 9B demonstrate improved clinical outcome in those patients in whom the treatment was applied at a concentration peak or strong increase trend of IL-12p70 and CD197/CD206.

Patterns of periodicity of sinusoidally fluctuating immune variables. Since a large proportion of time dependent profiles were fitted to cosine curves when a rather non-stringent criterion (the correlation coefficient) was used, only those data which fitted cosine curves with the value of R2 greater than the 75 percentile were selected. A similar technique was used for calculating cut-off value of the correlation coefficient: (a) the frequency distribution of the correlation coefficient was computed across profiles of all 14 variables analyzed; and (b) the value of the 75^{th} percentile (0.91) was accepted as a cut-off to eliminate profiles which did not fit a model well. As a result, seven profiles were eliminated where the cosine function period was longer than the observation time (14 days). Distinct rhythms were evident for the time-dependent fluctuation (days) of the corresponding plasma cytokine concentrations/cell counts. Table 4 shows the periods in days of the eight cosine curves which satisfied the selection criteria in this example. The shortest period is 3 days and all other periods except one are multiples of 3: 6, 9 and 12. One exception in this example is a 4 day period of IL12p70 in patient 1.

TABLE 4

| Patient | | CD197/ | IL- | IL- | CRP | CD11c/ | IL- | CD4/ |
|---|---|---|---|---|---|---|---|---|
| number | PFS | CD206 | 12p(70) | 17 | ng/mL | 14 | 1ra | 294 |
| 1 | 916 | 6 | 4 | | | | | |
| 4 | 748 | | | | | | | |
| 6 | 132 | | | | | 3 | | |
| 5 | 91 | | | | | | | 4 |
| 12 | 77 | | | 12 | | | 4 | |
| 10 | 70 | 12 | | | | | | |
| 7 | 68 | | | | | | | 3 |
| 2 | 37 | 3 | | 6 | 9 | | | |

The data in Table 4 show that distinct rhythms were evident for the time-dependent fluctuation (days) of the corresponding plasma cytokine concentrations/cell counts, specifically the ratio of polarized M1/M2 macrophages (CD197/CD206) (30), Interleukin-12 (IL-12p70), Interleukin-17 (IL-17), C-reactive protein CRP), CD11c-positive monocytes (CD11c/14) and Th2 helper T lymphocyte cell subset (CD4/294). For the majority of patients/variables, these rhythms followed a predictable pattern which was a multiple of 3 days (3, 6, 9 and 12 days, respectively) for most of plasma cytokines and cell counts. A few patients demonstrated a 4 day periodicity for IL-12p70, IL-1ra and CD4/294.

Determining the number and frequency of blood draws needed to accurately detect sinusoidal fluctuations in immune variables. The extrapolation of the obtained curves (FIG. 1C) for the time length of two periods (6, 12, 18 and 24 days correspondingly) demonstrated that every day sampling for at least 24 days would achieve an R square of 0.9 for cosine curve fitting. A data series collected with this frequency and for this period of time may allow more reliable analysis of the dynamics of those variables which fluctuate with amplitude not less then 45% of the mean value of the variable during the whole time of the observation (24 days). Only time-dependent concentration profiles with periods 12 days or shorter may be reliably analyzed under the described conditions. This analysis outlines the parameters of study design (frequency and duration of sample collection) necessary to directly test the hypothesis of the impact of timed chemotherapy delivery based on fluctuating immune variables (ongoing validation study).

Referring again to FIG. 1C, once the process extrapolates values for each of the selected immune variables, the process may compute the date(s) when the product Π achieves its maximum values for each of the selected immune variables within the extrapolated time period (142). The process next computes the dates when the maximum number of immune variables will have maximum values of the product Π (144). The process may report dates when the maximum number of immune variables will have maximum values of the product Π (146). These dates may correspond to a proposed day of treatment that has the best correlation with the patient's PFS.

The process may also output a report/table/plot of extrapolated and/or maximum values of Π products per variable for a period of 24 days after the last measurement, output a table of ranks or products per immune variable and output a plot of maximum values of product Π per variable for a period of 24 days after the last measurement (148).

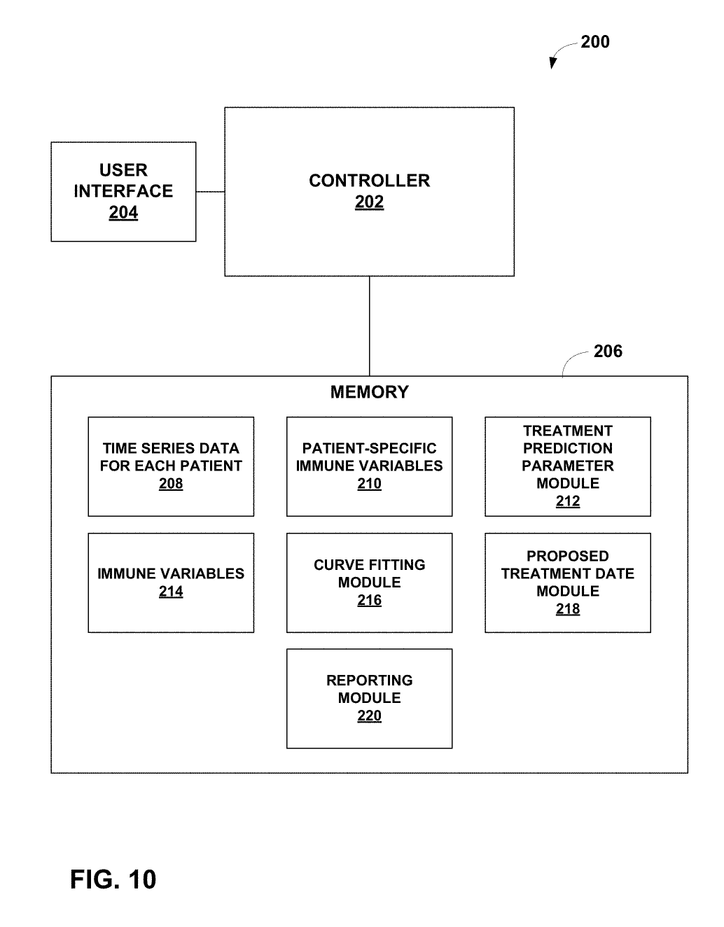

FIG. 10 is a block diagram illustrating an example system 200 for determination of favorable times for delivery of chemotherapy treatment. The system includes a controller 202 which processes the data and determines predicated favorable treatment times based on the biological parameter data for one or more patients. The system also includes a user interface 204 through which a user may input various process parameters and/or may view reports of the results of the analysis of time series data for one or more patients. The results may be presented in report format, and may include text, plots, graphs, charts, or other meaningful way of presenting the results. The user interface may also permit a user to input process parameters and/or data to be used by the system. A memory 206 stores the data and programming modules needed to analyze the time series data for one or more patients. For example, the memory may store the time series data for one or more patients 208, a list of the potential immune variables 214, and the patient-specific immune variables that fit a periodic function for that patient 210. The memory may also include a treatment prediction parameter module 212, a curve fitting module 216, a proposed treatment date module 218, and a reporting module 220 may generate reports regarding each patient's predicted favorable treatment times. These reports may be printed, transmitted to a local or remote computer and/or displayed on a local or remote computer.

The memory may also include programming modules such as a curve fitting module, a reporting module, a treatment prediction parameter (Π) module and a proposed treatment date module. Curve fitting module receives time series data of immune variable concentration for an observed time period for each of a plurality of identified immune variables and fits a periodic function to the time series data corresponding to each of the plurality of identified immune variables. Treatment prediction parameter module performs all of the calculations necessary to determine the treatment prediction parameter (Π), such as defining a relative concentration of the fitted periodic function, defining a relative derivative of the fitted periodic function and calculating the treatment prediction parameter based on the relative concentration and the relative differential.

Proposed treatment date module may choose the proposed treatment date such that the treatment prediction parameter (Π) is maximized. Reporting module may generate screen displays or printable reports including the proposed date of treatment that maximizes the treatment prediction parameter and/or other presentations of the raw data, intermediate data, or final results. The reporting module may allow the user to create customized reports depending upon the format and/or data the user wishes to view.

The system shown in FIG. 10 also includes a controller that, by following the programming modules stored in the memory, analyzes the time series data and determines proposed dates for timed delivery of chemotherapy as described herein.

The example study discussed herein describes the time-dependent (kinetic) relationship between the tumor and host immune response in 10 patients with metastatic malignant melanoma. The data analysis suggested that most biomarkers show a temporal variation, implying that these immune variables oscillate repeatedly, in an apparent predictable fashion. This is consistent with previously published reports of episodic “rhythmic” changes in hematology and immunobiology which follow a circadian (24 hour), infradian (greater than 24 hours—for example seven days or circaseptan), seasonal, or circannual (yearly) pattern. The use of single time point studies to describe the state of immune homeostasis in patients with cancer may be overly simplistic and potentially misleading. Therefore, the temporal variation of measured biomarkers and the pattern of change (and not only the degree of change itself) may better define an individual's response to illness.

The techniques described herein may provide evidence that rhythms exist in immune responses to malignant disease and suggest the possibility that such rhythms may be relevant to therapeutic success. Disruption of such biorhythms may have clinical consequences. These observations are consistent with the findings that patients with disorganized (non-curve-fitting) anti-tumor immune responses (see, e.g., FIG. 3) experienced a significantly decreased survival (PFS of 71 and 74 days, respectively), relative to those in whom the measured immune variables followed a predictable biorhythm (coefficient of correlation 0.72). In this example, it appeared that best clinical outcomes were observed in the two patients who best maintained a well synchronized anti-tumor immune response possibly overcoming global immune dysfunction of malignancy. Timed delivery of chemotherapy in that context may have allowed for a more precise therapeutic intervention leading to putative depletion of immune down-regulatory signals in favor of effective anti-tumor immunity.

In this example, distinct infradian rhythms were found in the fluctuations of most variables fitted to cosine functions which were in fact multiples of 3-4 days. The contribution of circadian variation to the fluctuation of immune variables was minimized in the example study by collection of blood samples at approximately the same time of day (between 8 and 10 AM); therefore, the rhythms observed in the example study are unlikely to be influenced by daytime/nighttime schedule.

By extrapolating the principle of chronotherapy to the anti-tumor immune response, it is possible that coupling treatment with these rhythms will improve the therapeutic index of cancer chemotherapy. It was originally posited that timed application of chemotherapy at a certain point in the immune cycle, based on the fluctuation of the CRP concentration, could selectively ablate the cycling suppressive elements of immunity, thus releasing the patient's immune system from down-regulation. However, the data (such as that presented herein) demonstrated no significant correlation between PFS and CRP concentration on the day of treatment. In this example, in order to accurately predict the fluctuation of the immune response and successfully time chemotherapy administration, one needs to consider not only the magnitude of change in concentration or immune cell frequency but also the dynamic change of a particular immune variable. In order to better characterize this time-dependent change, the analysis was extended to 29 other cytokines/chemokines/growth factors and 22 immune cell subsets and studied 1593 additional data points measured over 15 days in 10 patients with metastatic melanoma. By using mathematical modeling and curve fitting analysis a single parameter (Π) was defined that describes both the magnitude of change in concentration and the trend for increase or decrease of a given immune biomarker. This parameter may then be used to identify the variables for which application of chemotherapy at a distinct time-point in the immune cycle correlated with improved PFS.

CRP was initially an attractive candidate given its well established quantification methodology, ease of measurement, as well as previously described periodic fluctuations in healthy individuals as well as patients with chronic viral infections or cancer. The example data analysis, however, showed that there may be no correlation between CRP changes and clinical outcome (PFS) (correlation coefficient −0.60). Unexpectedly, two other variables, concentration of IL12p70 and the ratio of CD197/CD206 positive cells (ratio of polarized M1/M2 macrophages) exhibited satisfactory correlation with PFS in these examples, emerging as potential candidate biomarkers for timed administration of chemotherapy. Other biological variables, including some of those described herein, may also be appropriate biomarkers, depending at least in part upon the patient.

It shall therefore be understood that other immune variables not described herein may also, upon further study, exhibit satisfactory correlation with PFS, and that the disclosure is not limited in this respect.

The example study described herein shows that IL-12 fluctuates in a predictable pattern in patients with cancer (4 day period) and that application of TMZ therapy at a particular time-point when IL-12 is at a concentration peak or shows a strong positive trend (positive first derivative of the fitted function) may result in enhanced treatment effect and improved clinical outcome. The additional immunomodulatory properties of TMZ (in addition to its anti-tumor activity) may augment immunological responsiveness through destruction of regulatory T cells, disruption of homeostatic T cell regulation, or abrogation of other inhibitory mechanisms. Timed administration of this agent at a particular time-point in the immune response cycle when IL-12 shows a positive trend (2 out of the 4-day period), may selectively suppress Treg who lag behind T effectors in their clonotypic expansion. By that time, effector T cells may have proliferated and become activated and may be therefore less susceptible to the effects of TMZ chemotherapy.

In the example described herein, curve simulations using function parameters obtained in nonlinear regression fitting of cosine curves to the sample data with periods of 3 to 4 days. This simulation sought to (a) further assess the significance of curve fitting to experimental data; and (b) get a more accurate estimate of the minimum number of data points sufficient for reliable curve fitting, which may allow better planning for a future clinical trial.

Based on the extended example simulation data, an example list of candidate biomarkers, may include, for example, CRP, IL-10, IL-12p70, G-CSF, IL-9, VEGF, IL-1ra, IL-13, IL-15, IL-17, and immune cell subsets such as CD4/294, CD11c/14, CD197/CD206, CD206 and DR(hi).

In summary the data suggests that: (a) patients with stage IV melanoma exhibit a dynamic, not static, anti-tumor immune response; (b) an ordered pattern of change in plasma concentration of various cytokines/chemokines/growth factors and immune cell subsets was observed in patients with the longest PFS; (c) the fluctuations of most variables fit cosine functions with periods which are multiples of 3-4 days; and (d) delivery of cytotoxic therapy (TMZ) at a defined time in the biorhythmic immune oscillation appears to correlate with improved clinical outcome. The product between the relative concentration of an immune variable and the first derivative takes into consideration both the magnitude of the concentration and the dynamic trend of a given variable and could be used to guide personalized “timed” drug delivery. The data presented herein provide the basis for the design of experimental conditions for testing the hypothesis of timed chemotherapy delivery at a specific phase of the immune cycle.

In a more specific example, a cosine curve simulator (CCS) software module generates simulated cosine/sine curves using function parameters obtained in experiments measuring time-dependent concentration of a selected group of proteins in human blood samples. As discussed above, the simulator takes as an input time series measurements of concentrations of biological variables samples drawn from a number of patients. The other input is distribution of frequencies of technical errors of various magnitudes which was also measured in the experiment. The software outputs curves corresponding to 9 mathematical functions fitted to the input data series. Each fitted curve is supplemented with goodness of fit parameters. The software also outputs a table and a plot of probabilities of cosine curve detection as related to the amplitude, function period, frequency of sampling and length of the observation period.

One purpose of the CCS is to assess confidence bounds of the parameters of the data sets (period of observation, frequency of blood sampling, range of detectable periods of concentration fluctuation, range of detectable amplitudes of concentration fluctuation) for detection of data fitting to 9 mathematical functions.

The CCS algorithm may receive input as described above. The average value and standard deviation is calculated for each biological variable (concentration of a cytokine, chemokine, growth factor or a cell count of a specific cell type) across samples. A range of average+/−2 standard deviations is calculated for each parameter in the cosine function. There are 4 parameters in the cosine function f(x)=A+B*cos(C*x+D): parameter A determines the vertical shift of the curve, parameter B determines the amplitude, parameter C determines the period and D defines phase shift.

In one example, the range for parameter B is divided into 100 increments, and range for parameter C is divided into 20 increments to produce periods in the range from 1 to 20 days with 1 day increment. The CCS simulates a set of data points (which correspond to concentration of a protein or cell count) for all possible combinations of period and amplitude for each variable. Further, data may be simulated for three periods of observation: 10 day, 15 days and 20 days and for three frequencies of blood sampling: every day, every other day and with 1 to 2 day interval. Such a simulation will generate 936,000 data sets in total (52 variables*100 amplitudes*20 periods*3 observation periods*3 sampling frequencies). Collectively these data sets may be referred to as “Series A”. A signed experimental error is added to the ideal value of the function. The error value and frequency follows the distribution of error values obtained in the experiment and the sign is random.

R squared (R²) and standard error may be calculated for each simulated data set. The CCS generates a table and a histogram of distribution of frequencies of R². Further, CSS may generate another series of data sets—“Series B”. Each set of data points in this series may have the same combination of parameters (52 combinations of amplitude, period, observation period, sampling frequency. One combination per biological variable). However, in this example, the value of the function is not calculated by the cosine formula, but rather is a random number. This random number satisfies all above named parameters.

The curve-fitting as described above may then be applied to the simulated data. For example, curve-fitting may be applied to each data set to 9 mathematical functions (linear function, exponential function, exponential association, logistic model, Morgan-Mercer-Flodin (MMF) model, quadratic function, cosine function, rational function, Gaussian model) and reports which data sets fit any of the functions with R squared above 75^{th} percentile cut-off. The list of these data sets (IDs) may then uploaded into the CCS. Using “Series B” the CCS computes p-value for each simulated data set from the uploaded list. CSS outputs a table of simulated datasets with their parameters and associated p-values. These p-values represent the probability that a data set with a given combination of parameters is fitted uniquely to a cosine curve by chance alone.

A common problem for mathematical modeling of clinical data is the limited number of data points. Developing a model of a dynamic process requires a time series of measurements. Translated into the terms of a clinical setting this means blood or tissue samples collected with certain frequency over some period of time. It is common that the frequency and observation period allowed by the clinical standards are not sufficient to develop a mathematically sound model. For example, fitting protein concentration in blood measured six times during a period of two weeks to a cosine curve produces ambiguous results. Simulation and modeling study allows one to define experimental parameters to more reliably determine the function of a dynamic trend.

Fitting of 6 or 7 data points to a function with four parameters (sinusoidal and rational functions) is ambiguous even if the goodness-of-fit metrics are satisfactory (R² and coefficient of variation are close to 1.0, confidence interval is narrow, etc.). A straightforward way to resolve this ambiguity is to increase the number of data points. However, in a clinical setting this solution has strict limitations. In many situations human samples (blood or tissue) cannot be collected for long enough periods of time and frequently enough to obtain a time series of data points which would unambiguously satisfy stringent curve fitting criteria.

The techniques described herein may also determine sampling frequency, observation period, curve amplitude and period for one or more biological parameters that fit a function to within a desired goodness of fit. These sampling parameters may then be used to determine a schedule for the real-world collection of blood or tissue samples from patients that will be sufficient to adequately determine desired treatment times. Such a sample collection schedule results in a sufficient number of time points to arrive at a sufficiently accurate determination of desired treatment times while keeping the burden for patients as low as possible. In other words, given the maximum possible number of data points, determine sampling frequency, observation period, curve amplitude and period (for periodical function) which fit a function with high probability not by chance alone.

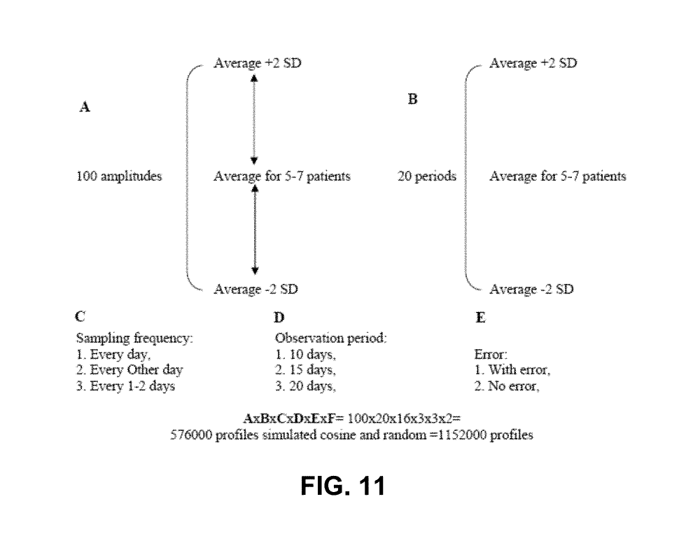

Time series of data points were simulated with input parameters derived from the example clinical data. FIG. 11 illustrates an example simulation which considered three different observation periods (10, 15 and 20 days), three various sampling frequency (every day, every other day and 1-2 days), one hundred amplitudes and twenty periods. In the example study, the following variables fitted cosine curves by defined selection criteria and had periods equal or shorter than 12 days: CD197/CD206 and IL12p70 (5 patients); CD4/294 and IL-15 (4 patients); CRP, IL-10, CD11c/14, CD206, IL-17, IL-13 (3 patients); IL-1ra, Il-9, G-CSF and VEGF (2 patients) and DR(hi) (one patient). Taking this into account, the amplitudes for a given variable were simulated as follows. The average of the parameter B, which defines the amplitude of the cosine function, was calculated across all patients in whom the time series for the variable fitted cosine curve. The interval B_{avg}+/−two standard deviations was calculated and divided into 100 fragments (see, e.g., FIG. 11.). Each of the 100 values of parameter B was used in the cosine equation to produce a profile with specific amplitude. Twenty different periods were simulated by the same technique. Each data series was simulated with or without experimental error. The error was calculated from the values of coefficient of variation maintaining the same distribution of error values as was obtained in the experiment. The error was added to or subtracted from the simulated value in random order. Time series for 16 variables which fitted cosine curve with R² above the 80 percentile cut-off in at least 7 out of 8 patients were simulated. Two sets of time series were simulated according to the described design. In the first set (Cosine profiles) concentration/cell count values were calculated by the cosine formula. In the second set (Random profiles) values were produced by the generator of random numbers within the set amplitude range. As result, 576000 data series of cosine profiles and 576000 data series of random profiles were obtained. All these profiles were fitted to the following five functions: logistic function, quadratic function, cosine function, rational function, Gaussian function, and MMF function (Morgan-Mercer-Flodin) and R² was recorded for each fitting.

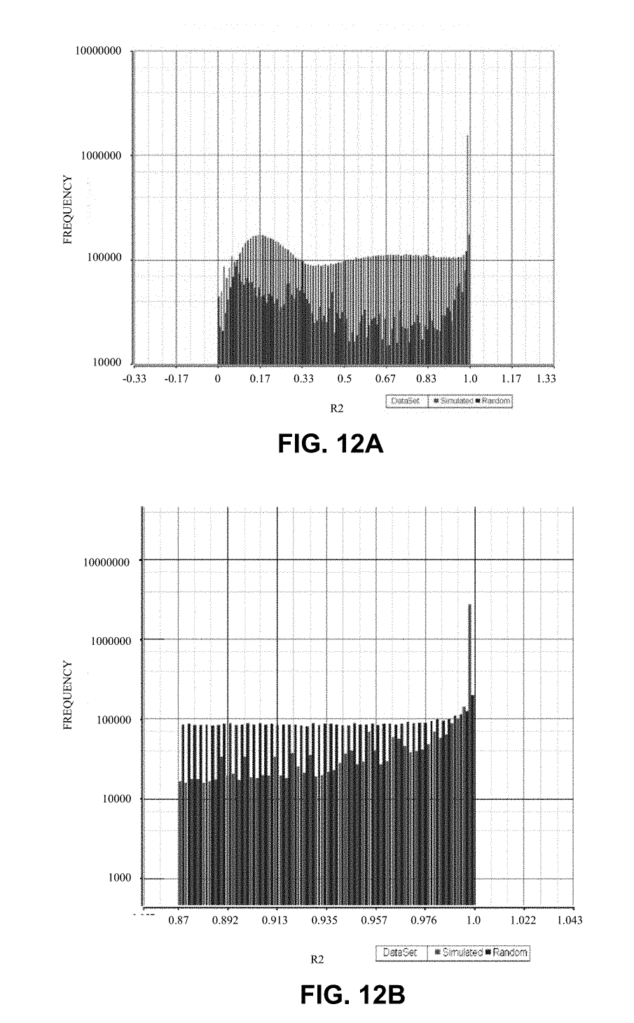

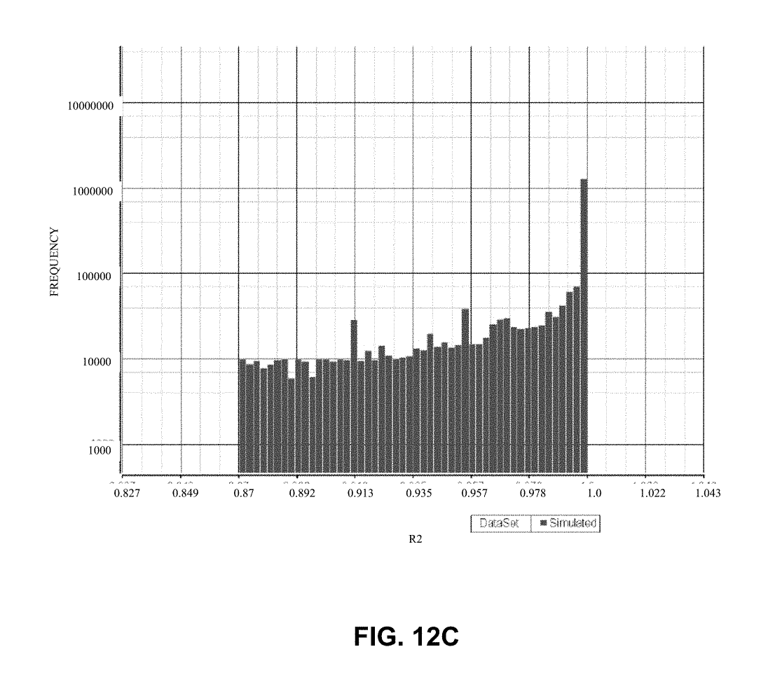

FIGS. 12A-12C are graphs illustrating the frequency distribution of R2 for various ranges and datasets. To determine potential clinical schedules for collection of data that would result in sufficiently accurate determination of desired treatment times, the proposed clinical schedules with multiple combinations of parameters were analyzed. The distribution of R² of the curve fitting in random and cosine data sets (see FIG. 12A) was computed and analyzed. Since the most of time series of measurements in original experiment fitted cosine curve, the properties of R² distribution for cosine function will now be described. The analysis of the R² distribution may permit identification of conditions (period, amplitude, sampling frequency, observation period, etc.) which predominantly produce true positive and true negative solutions as well as those which produce false positive and false negative solutions. A solution is the conclusion whether or not a time series of data points fits a cosine curve based on the value of R². Simulated profiles computed by the cosine formula produced true positive and false negative solutions when R² was high or low correspondingly. Likewise, random profiles produced false positive and true negative solutions. As a result, ranges of R² values corresponding to high sensitivity and specificity of the solutions can be determined. One of the goals of the simulation study was to determine the cutoff values of R² which allow one to achieve best combination of specificity and sensitivity.

A small number of time series (10185 profiles=0.0088% of the total number of profiles) formed straight lines and were excluded from further analysis. For the cosine profiles, about 81.7% (461998 out of 565821) of R² values lie in the range 0.980-1.0 (FIG. 12B). Of those, values obtained from fitting data series without introducing an error comprised 50%. The 90^{th} percentile of the R² values for the cosine profiles was 1.0 and 0.905 for the random profiles. The overall 90^{th} percentile of the R² values in the range from 0 to 0.98 was 0.87. R² values in the range from 0.87 to 1.0 were then considered. In one example, it may be reasonable to use the 90^{th} percentile of R2 subset as cut-off criteria for discriminating between random set of data points and those calculated by the cosine formula. This cutoff (rather than a more stringent 0.98) prevents having a larger number of false negative results. In other examples, other appropriate R2 cutoff could be used. The resulting subset of R² values contains ambiguous solutions (false positives and false negatives), the majority of which are introduced by profiles generated with observation period of 10 days and every other day blood sampling frequency. When all profiles generated with both of these conditions are removed, then only simulated cosine profiles fit cosine function with R² in the interval 0.8995 to 0.995 (FIG. 12C). No other tested observation period or sampling frequency produces significant number of R2 in this interval from random profiles.

As expected, the proportion of R² above the 90^{th} percentile cut-off obtained from fitting cosine profiles is higher for profiles with greater number of time points, that is, longer observation period or frequent blood sampling. This is a limiting factor in a clinical trial because blood samples cannot be taken during a long period of time with high frequency. This calls for an experimental design which would be a compromise between clinical requirements and demands of the curve fitting methods. Such a design is a sample collection schedule which allows a sufficient number of time points but keep the burden for patients as low as possible. A schedule satisfying these conditions is 5 sequential days when blood samples are collected, then 2 days of rest followed by another 5 days of sample collection. Such a collection schedule will be referred to herein as the “5-2-5 schedule.”

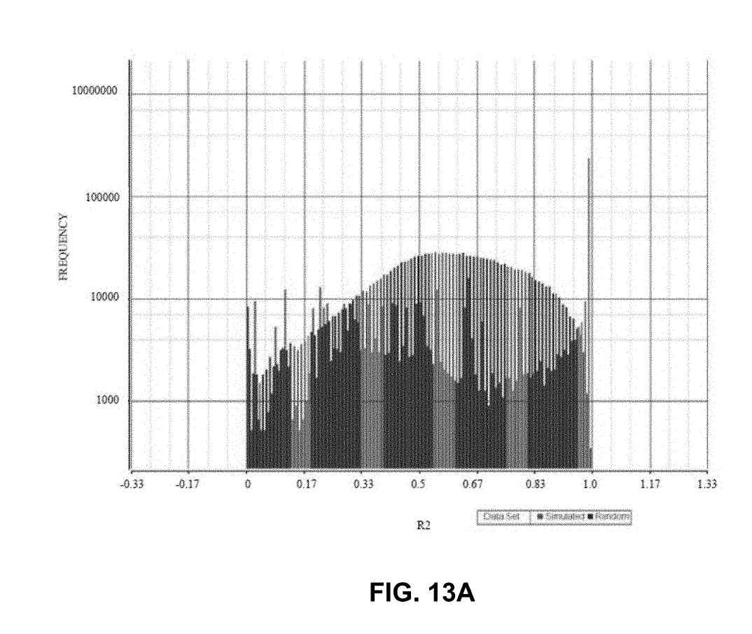

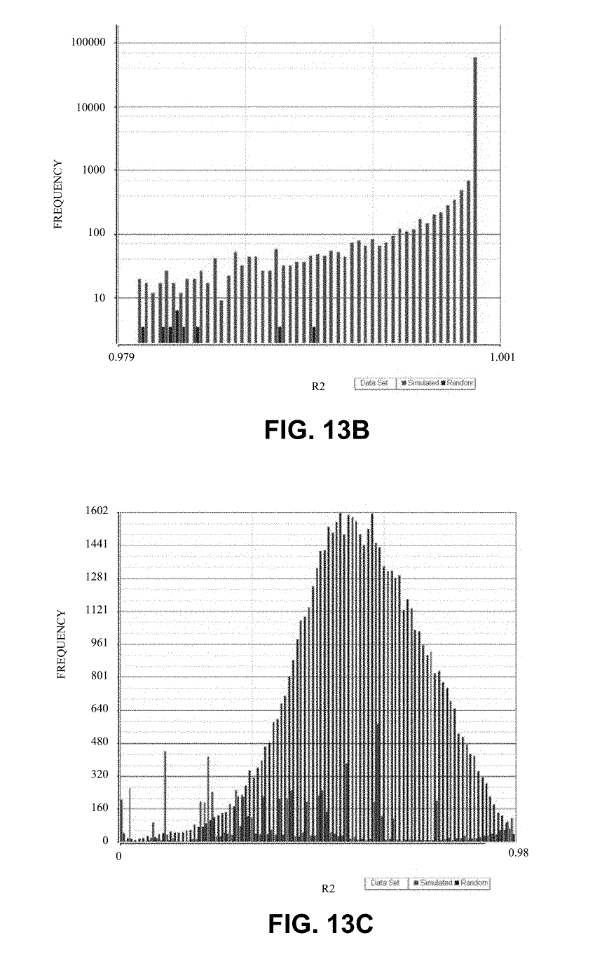

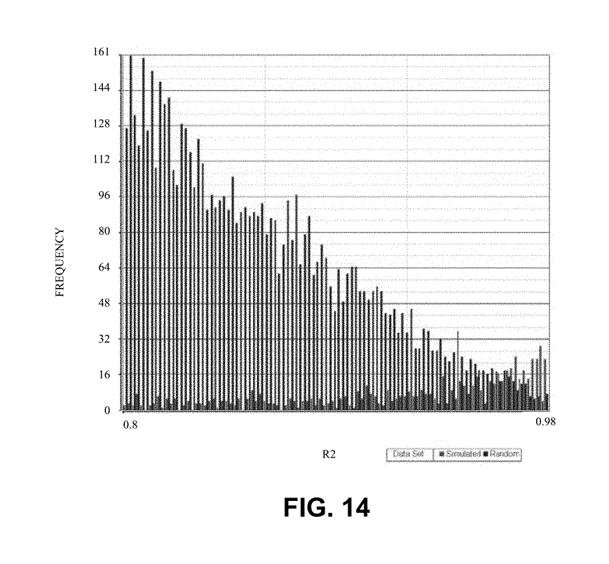

The 5-2-5 schedule gives 6 degrees of freedom for data fitting to a cosine function. Time series were simulated for this schedule. FIGS. 13A-13C are graphs illustrating the frequency distribution of R² for an example simulated 5-2-5 sample collection schedule. All R² values (56119 out of 56128) above 0.980 were generated by fitting simulated cosine profiles (FIG. 13A). The R² obtained from fitting the random profiles to the cosine function were largely prevalent in the range 0.000-0.980. The distribution of R² in this range is quasi-normal (FIG. 13C). The 90^{th} percentile of the subset of R² values in the range from 0 to 0.980 is 0.8055 (FIG. 13B). It follows, that if 90^{th} percentile is selected as a cut-off criteria for discriminating between random set of data points and those calculated by the cosine formula, then ambiguous solutions will lie in the R² value range from 0.8055 to 0.980 (FIG. 14). The receiver operating characteristic (ROC) analysis of 16 variables for this interval of R² values was determined. The best performing variable was IL-1ra (area under the curve (AUC)=0.955) and the worst performing variable was CRP (AUC=0.734) as shown in Table 5.

TABLE 5

| | |
|---|---|
| Variable | AUC |

| | |
|---|---|
| IL-1ra | 0.955 |
| IL-17 | 0.91 |
| CD197/CD206 | 0.886 |
| IL-9 | 0.884 |
| VEGF | 0.875 |
| CD11c/14 | 0.856 |
| IL-12p70 | 0.854 |
| CD206 | 0.844 |
| IL-10 | 0.844 |
| IL-13 | 0.837 |
| G-CSF | 0.824 |
| CD11c/CD123 | 0.806 |
| CD4/294 | 0.795 |
| IL-15 | 0.785 |
| DR (hi) | 0.778 |
| CRP | 0.734 |

Since the hypothesis in this example was that maximums of index Ø indicate active state of the immune response to tumorigenesis which is favorable for therapeutic treatment, the process may identify time periods when maximum number of variables have maximum cumulative value of index Ø. The process may account for variability of periods, increase and decrease rate of the change of immune variables such as concentration and cell counts as well as variability of the amplitude. Considering the intrinsic flexibility of a biological system in general, time periods corresponding to the set properties of immune parameters may be determined as intervals of time when the probability that immune parameters satisfy the set properties is elevated. Time intervals may be determined within the observation period as well as predicted in the future. The probability may gradually diminish in the vicinity of its peak value following normal or non-normal distribution.

Various methods can be used to identify the time periods of increased probability. In one example, a clustering algorithm, such as modified K-means clustering or other clustering algorithm, may be applied to find these time intervals for the time series generated in the 5-2-5 simulation. In this example, this method identified two days within a 12 day observation period when the cumulative index had maximum value. The same analysis may then be performed on the data obtained from patients with long PFS (916 days; Patient #1 and 841 days; patient #4) and short PFS (68 days; Patient #7 and 70 days Patient #10). Time series of three variables were clustered: concentration profiles of IL-1ra, IL-12p70 and counts of CD206+ cells for these four patients. Since time series obtained from the clinical trial had only 7 or 6 data points, 3 or 4 additional data points were extrapolated to match the same number of points (10) as were analyzed in the simulated 5+2+5 data set. The extrapolated values were computed using Fourier analysis. Clustering produced 1-3 days with maximum cumulative value of index Ø for each patient, as shown in Table 6. In another example, Markov Chain Monte Carlo (MCMC) method can be applied to identify time intervals when the probability that immune parameters satisfy the set properties is elevated. In this case, the random walk step of the MCMC is used to find the sought time intervals at a future time. In yet other examples, Bayesian methods or Multiobjective optimization can be applied to find these time intervals. It shall be understood, therefore, that the disclosure is not limited in this respect.

TABLE 6

| | | | Days | Minimum difference |
|---|---|---|---|---|
| Patient | | Treatment | predicted by | between treatment and |
| number | PFS | day | clustering | clustering days |
| 1 | 916 | 18 | 6, 21 | −3 |
| 4 | 841 | 11 | 13.5; 8.5 | −2.5 |
| 7 | 68 | 14 | 3.2; 9.8; 20.5 | 4.2 |
| 10 | 70 | 15 | 6.14 | 8.9 |
| 2 | 37 | 12 | 14, 6, 0.5 | −2 |
| 5 | 91 | 14 | 8.6 | 5.4 |
| 6 | 32 | 17 | 8.1 | 9 |
| 12 | 77 | 20 | 5.1, 24.2 | −4 |

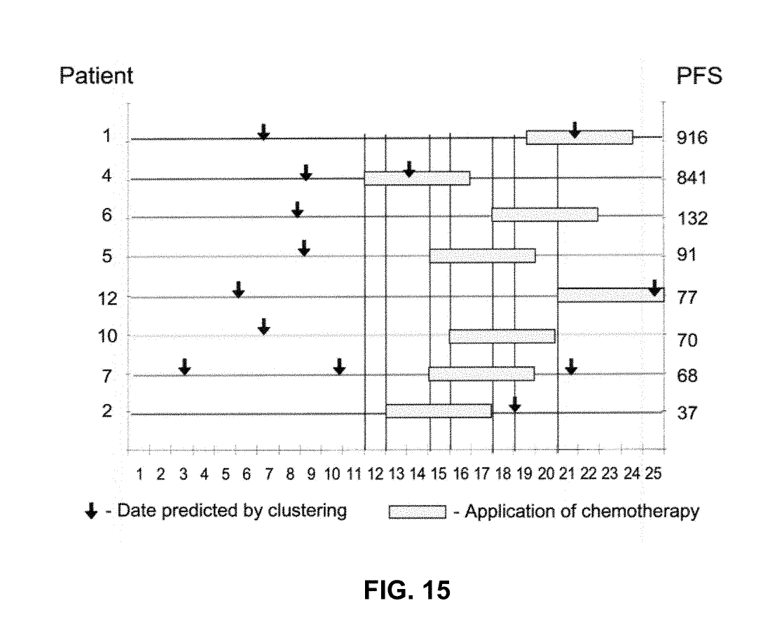

FIG. 15 is a chart illustrating the association between the 5-day period of actual chemotherapy application, time predicted by the example clustering algorithm and PFS in 8 melanoma patients. An example clustering method was applied to preliminary data obtained in a pre-clinical trial on 8 stage IV melanoma patients. Progression-free survival (PFS) time varied from 37 days to 916 days in these patients. Favorable time for chemotherapy application predicted with by the clustering algorithm fell within the 5-day period of chemotherapy application in two patients with the longest PFS (Patients #1 and #4). In all other patients except one, chemotherapy was applied several days before or after the days predicted by the clustering. In one patient, the day predicted by the algorithm fell on the last day of chemotherapy application (Patient #12).

It is noteworthy that treatment days were very close to the days identified by clustering in patients who had long PFS (Patients #1 and #4 in FIG. 15). In patients with relatively shorter PFS the treatment was delivered 6.6 (Patient #7) and 8.5 (Patient #10) days earlier than predicted by clustering (FIG. 15). Only profiles which fit cosine function with correlation coefficient greater than 0.86 were used. Based on this criterion IL-1ra was eliminated from clustering in Patients #1, 4 and 7 and the IL-12p70 profile was eliminated in Patient #10.

The techniques described herein for selecting one or more immune variables which may be as predictors of patient's response to pharmaceutical treatment, such as chemotherapy. The basic principle of the method is to accumulate and analyze the knowledge on performance of each of the measured variables in each patient in whom the measurements and the treatment were performed. This accumulation is achieved through creation of a database in which time series of measurements and progression-free survival (RFS) time are recorded. In some examples, the algorithm computes and enters into the database the R² value of the fitting of each time series to the cosine function. Next, frequency distribution of R² values is computed and the R² value of the 75^{th} percentile may be defined. This value serves as a cut-off for selecting variables in the next steps of the algorithm. Depending on required stringency of variable selection, a higher (or lower) R² cut-off level can be selected, for example, 80^{th} or 90^{th} percentile (or lower than 75th percentile).

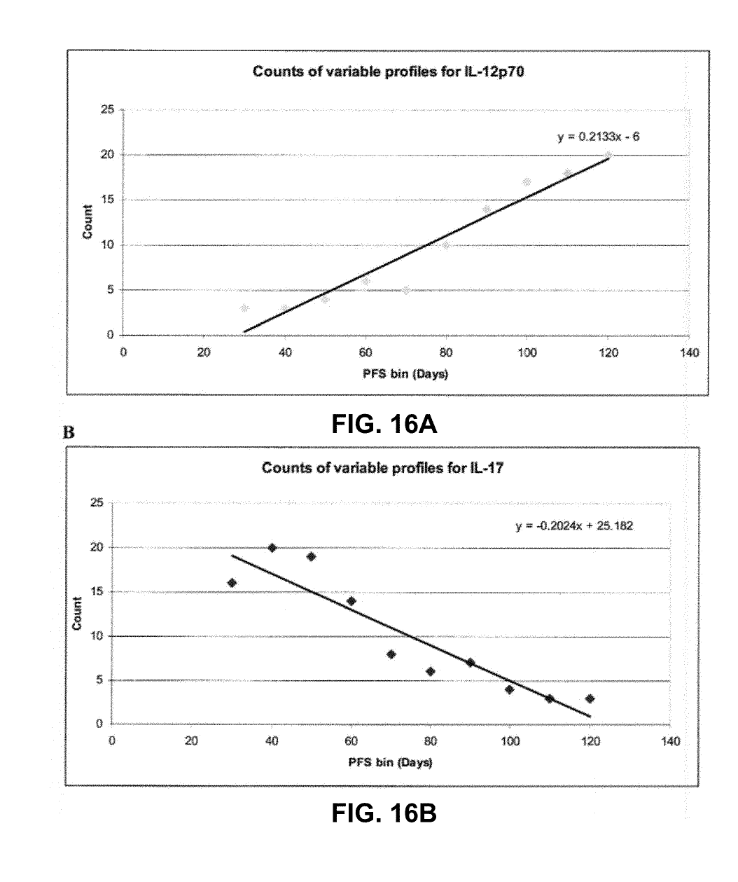

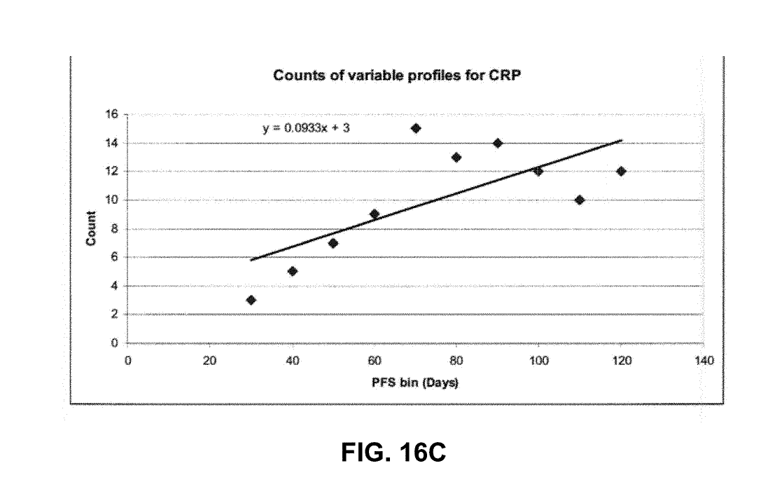

In another example, in order to select immune variables to be used as discriminators in the clustering algorithm, the algorithm may divide the whole range of PFS longevities into the number of bins ten times less than the number of patients. For each bin the algorithm counts profiles of each variable with R² above the cut-off value and the sum of Π indices on the treatment start date for these variables (see, e.g., Table 7 and Table 8). Next, the linear regression analysis is performed both on the counts of each variable with R² above the cut-off value and on the sums of Π indices and the slope of the regression line is computed. Variables with high positive value of the sum of the slopes (for example, IL-12, IL-1ra and CD206 in Table 7) have positive correlation (PC) with PFS (see, e.g., the graph for IL-12p70 in FIG. 16A), variables with high negative sum of the slopes (for example, IL-17 and IL-10 in Table 7) have negative correlation (NEC) (see, e.g., the graph for IL-17 in FIG. 16B), and variables with sum of the slopes close to zero (for example, IL-13, IL-15 and CRP in Table 7) have no correlation (NOC) with PFS (see, e.g., the graph for CRP in FIG. 16C). In this example, the cut-off for PC variables is the 75^{th} percentile (mean+0.67× Standard Deviation) of all sum values and for the NEC the cut-off is the 25^{th} percentile (mean−0.67×Standard Deviation). Alternatively, to decrease the stringency of the variable selection either cut-off of the slopes for only regression line of the counts, or only slopes for sums of Π indices can be considered.

TABLE 7

| | | | | | | | | | | | Num of | | | |
|---|---|---|---|---|---|---|---|---|---|---|---|---|---|---|
| Variable ↓ | Counts of variable profiles | | | | | | | | | | patients | Slope | Mean | SD |
| IL-12 | 3 | 3 | 4 | 6 | 5 | 10 | 14 | 17 | 18 | 20 | 100 | 2.13 | 0.51 | 1.80 |

| | | | | | | | | | | | | | |
|---|---|---|---|---|---|---|---|---|---|---|---|---|---|
| IL-13 | 8 | 9 | 10 | 6 | 8 | 15 | 10 | 12 | 9 | 13 | 100 | 0.45 | 75^{th} percentile |
| IL-15 | 8 | 13 | 10 | 9 | 10 | 12 | 9 | 12 | 8 | 9 | 100 | −0.09 | 1.72 |
| IL-17 | 16 | 20 | 19 | 14 | 8 | 6 | 7 | 4 | 3 | 3 | 100 | −2.02 | |
| IL-10 | 18 | 20 | 16 | 12 | 10 | 8 | 6 | 3 | 3 | 4 | 100 | −2.00 | |
| IL-1ra | 2 | 3 | 4 | 3 | 8 | 9 | 10 | 19 | 22 | 20 | 100 | 2.37 | |
| CD206 | 2 | 2 | 4 | 5 | 7 | 10 | 12 | 17 | 20 | 21 | 100 | 2.34 | 25^{th} percentile |
| CRP | 3 | 5 | 7 | 9 | 15 | 13 | 14 | 12 | 10 | 12 | 100 | 0.93 | −0.69 |

| | | | | | | | | | | |
|---|---|---|---|---|---|---|---|---|---|---|
| PFS bin→ | 30 | 40 | 50 | 60 | 70 | 80 | 90 | 100 | 110 | 120 |

TABLE 8

| Variable ↓ | Sum of Π indices | | | | | | | | | | Total | Slope | Mean | SD |
|---|---|---|---|---|---|---|---|---|---|---|---|---|---|---|
| IL-12 | 10 | 11 | 17 | 19 | 33 | 62 | 74 | 93 | 138 | 157 | 614 | 16.90 | 4.63 | 12.60 |

| | | | | | | | | | | | | | |
|---|---|---|---|---|---|---|---|---|---|---|---|---|---|
| IL-13 | 12 | 17 | 22 | 34 | 27 | 42 | 67 | 87 | 112 | 142 | 562 | 13.78 | 75^{th} percentile |
| IL-15 | 15 | 14 | 23 | 17 | 19 | 21 | 16 | 20 | 18 | 19 | 182 | 0.29 | 13.07 |
| IL-17 | 196 | 173 | 152 | 163 | 110 | 83 | 63 | 54 | 37 | 22 | 1053 | −20.20 | |
| IL-10 | 63 | 67 | 54 | 57 | 62 | 68 | 59 | 57 | 61 | 64 | 612 | −0.04 | |
| IL-1ra | 34 | 47 | 59 | 72 | 84 | 98 | 124 | 157 | 178 | 205 | 1058 | 18.90 | |
| CD206 | 13 | 12 | 17 | 22 | 26 | 32 | 43 | 52 | 57 | 68 | 342 | 6.40 | 25^{th} percentile |
| CRP | 23 | 34 | 42 | 54 | 52 | 48 | 53 | 47 | 41 | 34 | 428 | 1.00 | −3.81 |

| | | | | | | | | | | |
|---|---|---|---|---|---|---|---|---|---|---|
| PFS bin→ | 30 | 40 | 50 | 60 | 70 | 80 | 90 | 100 | 110 | 120 |

TABLE 9

| | Slope for the | Slope for | | | |
|---|---|---|---|---|---|
| | number of | the sum of | | | |
| Variable | counts | PI | Sum | Mean | SD |
| IL-12 | 2.13 | 16.90 | 19.03 | 5.14 | 14.0 |

| IL-13 | 0.45 | 13.78 | 14.23 | 75^{th} percentile |
|---|---|---|---|---|
| IL-15 | −0.09 | 0.29 | 0.21 | 14.56 |
| IL-17 | −2.02 | −20.20 | −22.22 | |
| IL-10 | −2.00 | −0.04 | −2.04 | |
| IL-1ra | 2.37 | 18.90 | 21.27 | |
| CD206 | 2.34 | 6.40 | 8.74 | 25^{th} percentile |
| CRP | 0.93 | 1.00 | 1.93 | −4.28 |

Tables 7-9 illustrate data corresponding to example procedures that may be used to select immune variables that will may used as discriminators in the clustering algorithm. The range of PFS time is divided into a number of bins (clusters) 10 times less than the number of patients. In this example there were 100 patients and so the PFS times were divided into 10 PFS bins (see, e.g., the last row of Table 7).

Temporal profiles which fit the cosine function with R² greater than selected cut-off are counted for each RFS bin and the slope of the regression curve of the counts is computed. Table 7 shows the mean and standard deviation (SD) of the slope values for all variables. These are used to calculate the 75^{th} percentile (mean+0.67×Standard Deviation) and the 25^{th} percentile (mean−0.67× Standard Deviation) of the slope values. In this example, variables for which the slope values were above the 75^{th} percentile include IL-12, IL-1ra, and CD206. Variables for which the slope values were below the 25^{th} percentile include IL-17 and IL-10.

Table 8 shows the sums of Π indices on the first treatment day for temporal profiles which fit the cosine function with R² greater than selected cut-off are computed for each RFS bin and the slope of the regression curve of the sums is computed. The mean and standard deviation (SD) of the slope values for all variables are computed and are used to calculate the 75^{th} percentile (mean+0.67× Standard Deviation) and the 25^{th} percentile (mean−0.67× Standard Deviation) of the slope values. In this example, variables for which the slope values were above the 75^{th} percentile include IL-12, IL-13 and IL-1ra. Variables for which the slope was below the 25^{th} percentile include IL-17.

Table 9 shows the sum of the slope values computed in Table 7 and Table 8 for each variable. The mean and standard deviation (SD) of the sums for all variables are computed and are used to calculate the 75^{th} percentile (pink) and the 25^{th} percentile (blue) of the slope values. In this example, variables for which the sum of the two slope values were above the 75^{th} percentile include IL-12 and IL-1ra. Variables for which the sum of the two slopes that were below the 25^{th} percentile include IL-17.

Variables with slopes above the cut-off value(s) identified in any one or more of the sums shown in Table 7, Table 8 or Table 9 may be used as discriminators in the clustering algorithm.

In addition, although the examples given herein include those variables with positive correlation, those variables having negative correlation may also be taken into account. For example, reciprocal changes in positive and negative correlated variables may be expected. That is, for those biologic variables with negative correlation, the process may want to treat when they are at lower concentration, low abundance, or showing a declining trend, for example.

Time-dependent fluctuations' profiles of the selected immune variables are used to determine the optimum time of chemotherapy delivery by using the following method. Cosine profiles of the fluctuations may be clustered with the aim to find time window, during which the frequency of peak values of the index Ø is the highest. The clustering is done by the K-means method with modifications. K-means clustering requires a priori knowledge of the number of clusters in which the objects (profiles) will be grouped. By this method, the number of groups is determined from the number of full function periods which fit into one observation period. The maximum possible number of groups equals the maximum number of function periods and the minimum number of groups equals the minimum number of function periods which fit into one observation period. The algorithm computes the number of clusters for the whole range of integers from the maximum to the minimum numbers. For each iteration (number of clusters) and for each variable the algorithm calculates the dates when the Π index has maximum value. These dates are used as centroids for K-means clustering. Since the result of K-means clustering depends on the order of initial centroids, the example modification performs clustering for all possible combinations of centroids and then computes the date when the sum of indices for all clustered cosine profiles was maximal. Next, the algorithm computes the dates with maximum sum of relative Π indices across all possible combination of centroids and all numbers of clusters. These dates are outputted as favorable dates for chemotherapy application for a given patient and a given set of immune variables (FIG. 2).

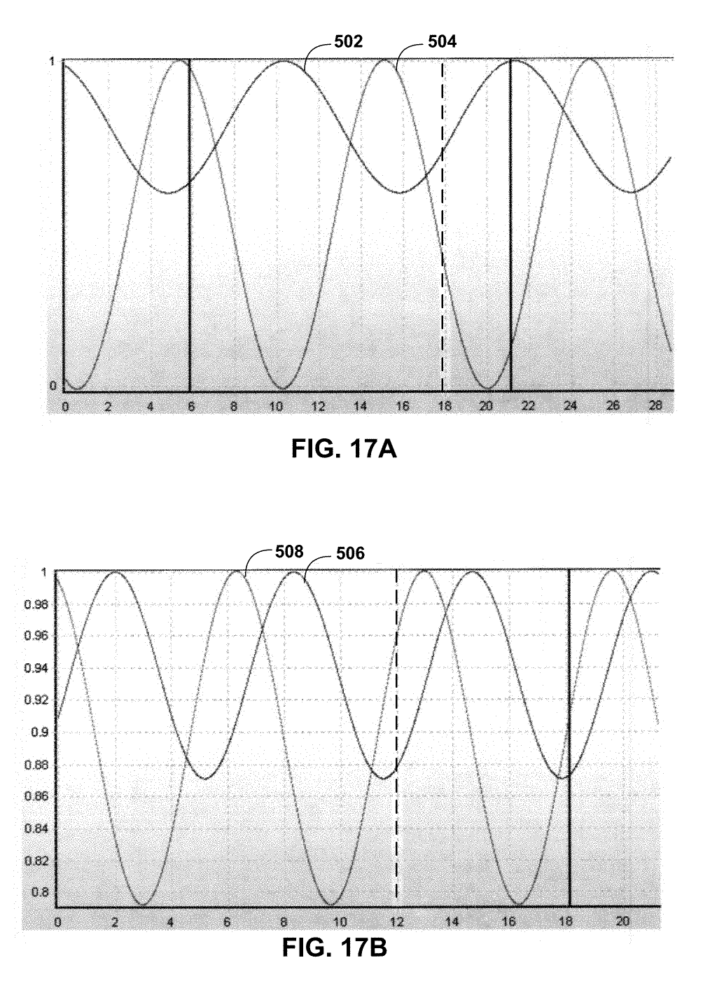

FIGS. 17A and 17B are graphs illustrating example clustering of concentration profiles IL-1ra (502) and IL-12p70 (504) in Patient #1 (PFS=916 days) (FIG. 17A) and concentration profiles IL-1ra (506) and IL-12p70 (508) in Patient #2 (PFS=37 days) (FIG. 17B). Black vertical lines represent dates, predicted by the clustering algorithm; dashed vertical lines represent dates when chemotherapy was started. In this example, three variables were clustered, but profiles for only two variables are shown on the plots for each patient. This resulted from filtering out profiles which did not satisfy the threshold criteria (in this case the goodness-of-fit criterion (R² value)) for a specific variable in an individual patient. The corresponding graph illustrating the association between the 5-day period of chemotherapy application, time predicted by the clustering algorithm and progression-free survival time in 8 melanoma patients is shown in FIG. 15.

Although in FIGS. 17A and 17B the variables used to determine treatment time(s) are the same (e.g., IL-1ra and IL-12p70) for each of the two patients, it shall be understood that this need not be the case. For example, the analysis may determine that for certain patients only one immune variable satisfies the threshold criteria, while for other patients two or more immune variables may satisfy the threshold criteria. In addition, the immune variables satisfying the threshold criteria may be different for different patients. The determination of favorable treatment times may therefore be patient-specific in the sense that only those biological variables satisfying desired threshold values may be used to determine favorable treatment times for each individual patient.

The example systems and/or methods described herein analyze time-dependent fluctuations of at least one biological variable measured in blood samples obtained from clinical patients and determine one or more favorable times for the pharmacological treatment of the patient. The systems and/or methods determine favorable time(s) for chemotherapy delivery based on serial measurements of one or more biological variables. In some examples, the biological variables are immune variables.

Each new series of experimental measurements may be processed according to the described workflow. This iterative computation of simulated parameters based on ever growing experimental evidence may iteratively enhance statistical power accuracy of p-values and overall precision in detecting functions to which the data fits. This, in turn, may enhance the accuracy of prediction of one or more favorable date(s) for chemotherapy treatment.

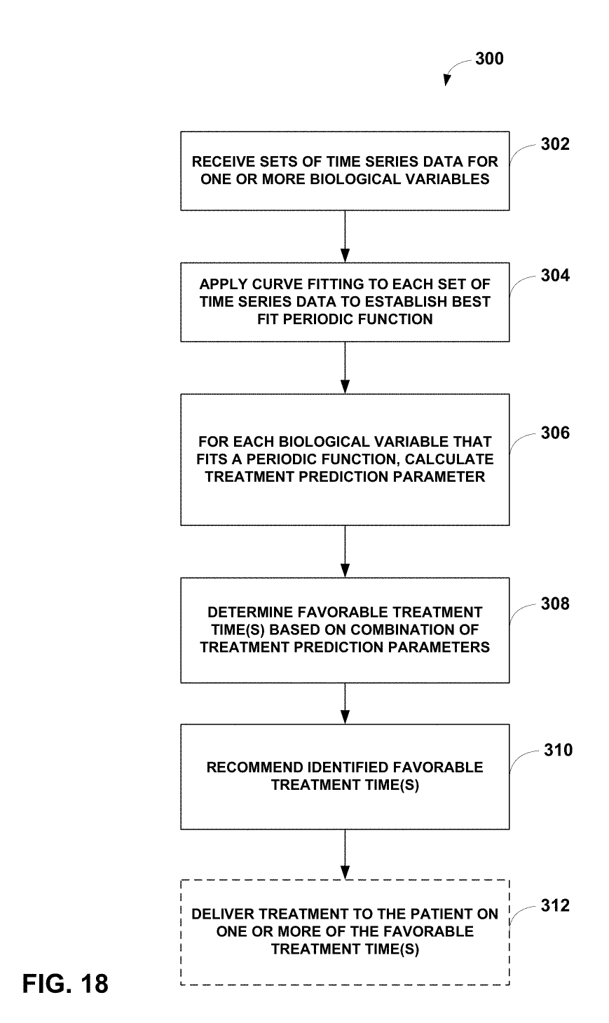

FIG. 18 is a flowchart illustrating an example process 300 by which a controller, such as controller 202 of system 200 shown in FIG. 10, may determine favorable treatment time(s) for delivery of chemotherapy treatment (or other type of pharmacological treatment) in a patient. The controller may receive sets of time series data for one or more biological variables (302). The biological variables may include, for example, immune variables. The immune variables may include, for example, IL-10, IL-12p(70), G-CSF, IL-9, VEGF, CD206, IL-1ra, IL-13, IL-15, IL-17, CD4/294, CD11c/14, CD197/CD206, and/or DR(hi). However, other immune or biological variables may also be included, and the disclosure is not limited in this respect.

The controller may apply curve fitting to each set of time series data to establish a best fit periodic function (304), if any. That is, the controller may determine whether each set of time series fits a periodic function. The controller may also determine the best-fit periodic function, if any, for each set of time series data. The periodic function may include, for example, a sinusoidal function, such as a sine or cosine function, any of the periodic functions described herein, or any other periodic function. For each biological variable that fits a periodic function, the controller may calculate a treatment prediction parameter (for example, the parameter or index Ø as described herein) (306). The treatment prediction parameter may be based on, for example, the relative concentration of the biological variable and the relative derivative of the best fit periodic function. The controller may determine one or more relatively more favorable treatment time(s) based on a combination of the treatment prediction parameters (308). For example, the controller may sum or otherwise combine the treatment prediction parameters to arrive at a combined treatment prediction parameter. The controller may further generate a report, display, or otherwise communicate a recommendation as to the one or more identified favorable treatment times to deliver the treatment to the patient (310). In some examples, the treatment may be delivered to the patient on one or more of the identified favorable treatment time(s) (312).

In another example, the determination of one or more favorable dates for delivery of therapeutic treatment to a specific patient is based on the “state” of one or more biological variables of the patient on the proposed treatment date(s). In this example, the predicted state of a biological variable on a proposed date of treatment refers to whether the concentration of the biological variable is predicted to be greater than a threshold value on the proposed date of treatment (state=HIGH or UP), or less than the threshold value on the proposed date of treatment (state=LOW or DOWN).

Predicting “states” of one or more biological variables may result in more accurate prediction of a favorable day for therapy as the actual concentration of the biological variable need not be predicted; rather, only the state of the periodic pattern (UP or DOWN) needs to be predicted. This may result in higher accuracy based on a reasonable number of data points (e.g., the number of daily blood draws from oncological patients) achievable with real world patients. In this example, therefore, one or more date(s) favorable for delivery of therapeutic treatment to a patient may be based on the determination of whether the states of certain biological variables are High (UP) or Low (DOWN) on the proposed treatment dates, rather than on predicting the concentration of the biological variables on the proposed treatment dates. Hence, more accurate prediction of a favorable time for therapy may be achieved with a smaller number of data points.

Each biological variable may have an associated threshold value. In other words, the threshold value may be different for each biological variable. Thus, the predicted state of a particular biological variable on a proposed date of treatment refers to whether the concentration of the biological variable is predicted to be greater than a threshold value associated with that biological variable on the proposed date of treatment (state=HIGH or UP), or less than the threshold value associated with that biological variable on the proposed date of treatment (state=LOW or DOWN).

In this manner, as one example, a method of cancer treatment may include administering chemotherapy treatment to a patient on a favorable treatment date identified based on a predicted state of at least one biological variable in the blood of the patient on the favorable treatment date. As another example, a method of cancer treatment may include administering chemotherapy treatment to a patient on a favorable treatment date identified based on predicted states of a plurality of biological variables in the blood of the patient on the favorable treatment date.

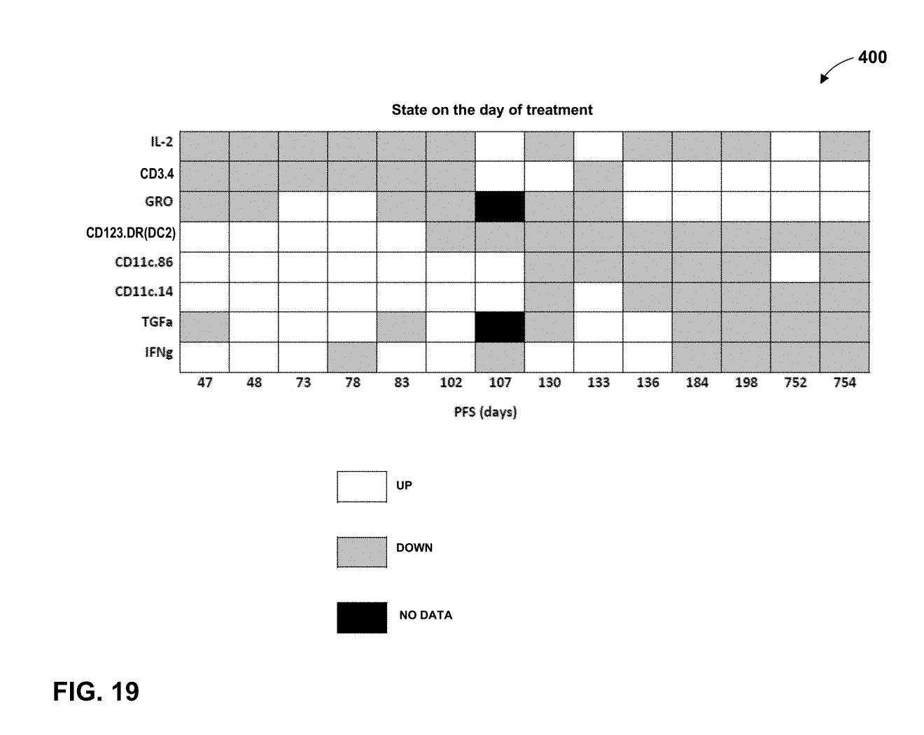

FIG. 19 is a graph illustrating the states of selected biological variables vs. progression-free survival (PFS) on the day of therapy administration for 14 patients in a clinical trial. FIG. 19 illustrates that patients having the highest progression free survival correlated with a first set of biological variables having a concentration state of “UP” on the day of therapeutic treatment and a second set of biological variables having a concentration state of “DOWN” on the day of therapeutic treatment. In this example, the first set of biological variables includes CD3.4 and GRO and the second set of biological variables includes IL-2, CD123.DR(DC2), CD11c/86, CD11c/14, TGFa, and IFNg. It shall be understood, however, that other biological variables may also be included in either the first or the second set of biological variables if more or different data were available.

In general, the disclosure is directed to the idea that progression free survival may be correlated with one or more conditions. One such condition may include that favorable date(s) for delivery of therapeutic treatment correspond to proposed dates on which a maximum number of a first set of biological variables have a concentration state of UP on the proposed day of therapeutic treatment. Another such condition may include that favorable dates for delivery of therapeutic treatment correspond to proposed dates on which a maximum number of a second set of biological variables have a concentration state of DOWN on the proposed day of therapeutic treatment. These conditions may be referred to as “favorable states” for delivery of treatment to the patient.

In addition, favorable date(s) for delivery of therapeutic treatment may also correspond to dates on which a minimum number of the first set of biological variables have a concentration of DOWN on the proposed day of therapeutic treatment and/or a minimum number of the second set of biological variables have a concentration state of UP on the day of therapeutic treatment. These conditions may be referred to as “unfavorable states” for delivery of treatment to the patient. One or more favorable date(s) for delivery of a therapeutic treatment may be identified by maximizing the number of favorable states and/or minimizing the number of unfavorable states specific to the patient.

Cluster or classification analysis, or other technique known to those of skill in the art, may be used to find combinations of the maximum number of favorable states of biological variables and/or a minimum number of unfavorable states.

In a retrospective analysis of data obtained in a clinical trial, a percentage of correctly predicted states across 19 patients for 50 cytokines and cells which satisfied the biomarker criteria technically reproducible and have periodical profiles in at least 50% of the patients was calculated. The average rate of correctly predicted states in this example was 69% as opposed to 40% of correct predictions with curve fitting methods.

A Cox proportional hazards analysis was applied to assess the correlation between the state of each of the selected 50 potential biomarkers with PFS of the patients. The correlation was statistically significant for the following 8 biomarkers: CD3.4 (p-value=0.0018); CD11c.14 (p-value=0.014); GRO (p-value=0.0177); IFNg (p-value=0.015); TGFa (p-value=0.0068); IL-2 (p-value=0.03); CD11c.86 (p-value=0.047), CD123.DR (p-value=0.0001. The states for each of these biological variables from this example data set are shown in FIG. 19.

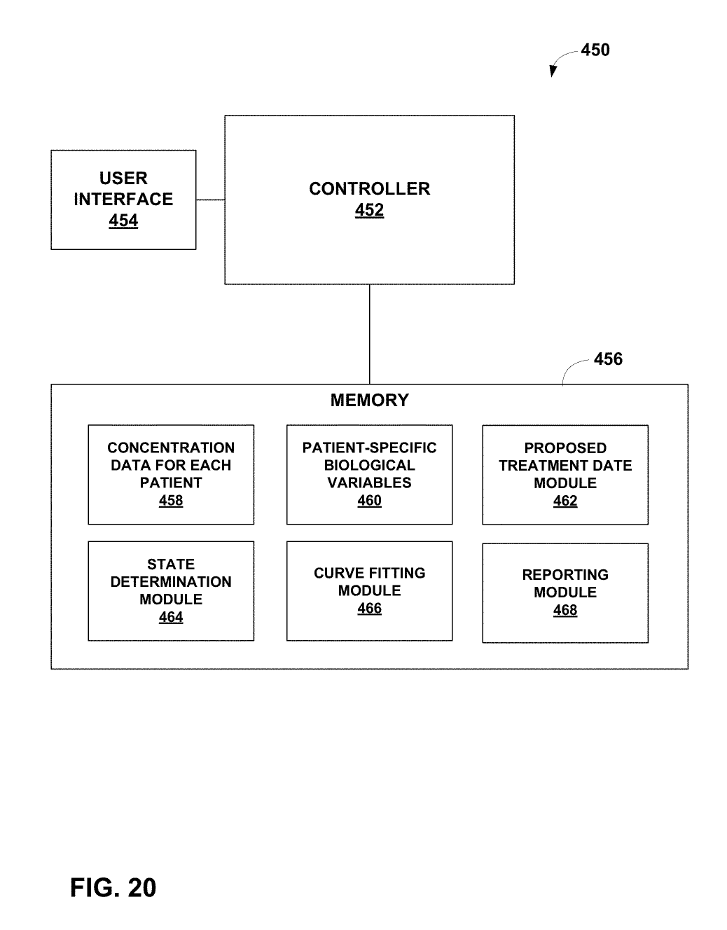

FIG. 20 is a block diagram illustrating an example system 450 that determines one or more favorable dates for delivery of pharmacological or other therapeutic treatment based on an analysis of the states of one or more biological variables on the proposed dates of treatment. In one example, the biological variables may be one or more lymphocyte subtypes and/or one or more monocyte subtypes. In another example, system 450 may also determine one or more favorable dates for delivery of pharmacological or other therapeutic treatment based on a lymphocyte-to-monocyte ratio on the proposed dates of treatment.

System 450 includes a controller 452 (including one or more processors or other computing elements) which processes the biological variable data for a patient and determines one or more favorable treatment dates to deliver a therapeutic treatment to the patient. The system may include a user interface 454 through which a user may input various process parameters and/or may view reports of the results of the analysis of time series data for one or more patients. The results may be presented in report format, and may include text, plots, graphs, charts, or other meaningful way of presenting the results. The user interface may also permit a user to input process parameters and/or data to be used by the system.

A memory 456 or other computer readable storage media stores the data and programming modules that, when executed by the controller 452, analyze the data corresponding to concentrations of one or more biological variables in blood samples from of one or more patients and determine one or more favorable dates for delivery of pharmacological or other therapeutic treatment for each patient. For example, memory 456 may store the concentration data 458 corresponding to concentrations of one or more biological variables in the blood samples for each patient. Memory 456 may also include data identifying the patient-specific biological variables for which a periodic function was fitted for each patient 460. Memory 456 may also include a curve fitting module 466, a proposed treatment date module 462, and a reporting module 468. Reporting module 468 may generate reports regarding each patient's predicted favorable treatment times. These reports may be printed, transmitted to a local or remote computer and/or displayed on a local or remote computer.

Curve fitting module 466 includes computer-readable instructions that, when executed by controller 452, permit the controller 452 to analyze the concentration data for each biological variable obtained for each patient over an observed time period and fit the data to a periodic function. The periodic function may include, for example, a sinusoidal function, such as a sine or cosine function, any of the periodic functions described herein, or any other periodic function. Curve fitting module 466 further includes computer-readable instructions that, when executed by controller 452, permit the controller 452 to extrapolate the fitted periodic function to a plurality of proposed treatment dates occurring subsequent to the observed time period. Curve fitting module 466 may use a Levenberg-Marquardt (LM) algorithm, or LM augmented with extended Kalman filter or with unscented Kalman filter or any other method of fitting and extrapolating the periodic function. The fitted periodic functions may be different for the different biological variables (e.g., different sinusoidal functions having different periods and/or amplitudes). Thus, each biological variable may have an associated fitted periodic function.

State determination module 464 includes computer-readable instructions that, when executed by controller 452, permit the controller 452 to determine the state of the biological variable for each of the plurality of proposed treatment dates based on the extrapolated fitted periodic function. In this example, the “state” of the biological variable refers to whether the concentration of the biological variable in the patient is “UP” on the proposed date of treatment, or “DOWN” on the proposed date of treatment. The purpose is to predict the state of the biological variable at future days on which treatment may be delivered.

In another example, state determination module 464 includes computer-readable instructions that, when executed by controller 452, permit the controller 452 to determine a lymphocyte-to-monocyte ratio for each of the plurality of proposed treatment dates based on the extrapolated fitted periodic function.

Proposed treatment date module 462 includes computer-readable instructions that, when executed by controller 452, permit controller 452 to determine one or more favorable treatment date(s) for a patient based on the states of the patient-specific biological variables. In one example, the proposed treatment date module 462 may identify date(s) with a maximum number of favorable states. In another example, the proposed treatment date module 462 may identify date(s) with a maximum number of favorable states and/or a minimum number of unfavorable states. The date(s) when these conditions are met may be recommended as the one or more favorable dates for delivery of the pharmacological or other therapeutic treatment to the patient.

In another example, proposed treatment date module 462 includes computer-readable instructions that, when executed by controller 452, permit controller 452 to determine one or more favorable treatment date(s) for a patient based on lymphocyte-to-monocyte ratio(s) on the one or more proposed treatment dates.

Reporting module 468 may generate screen displays or printable reports including the one or more proposed date(s) of treatment and/or other presentations of the raw data, intermediate data, or final results. The reporting module may allow the user to create customized reports depending upon the format and/or data the user wishes to view.

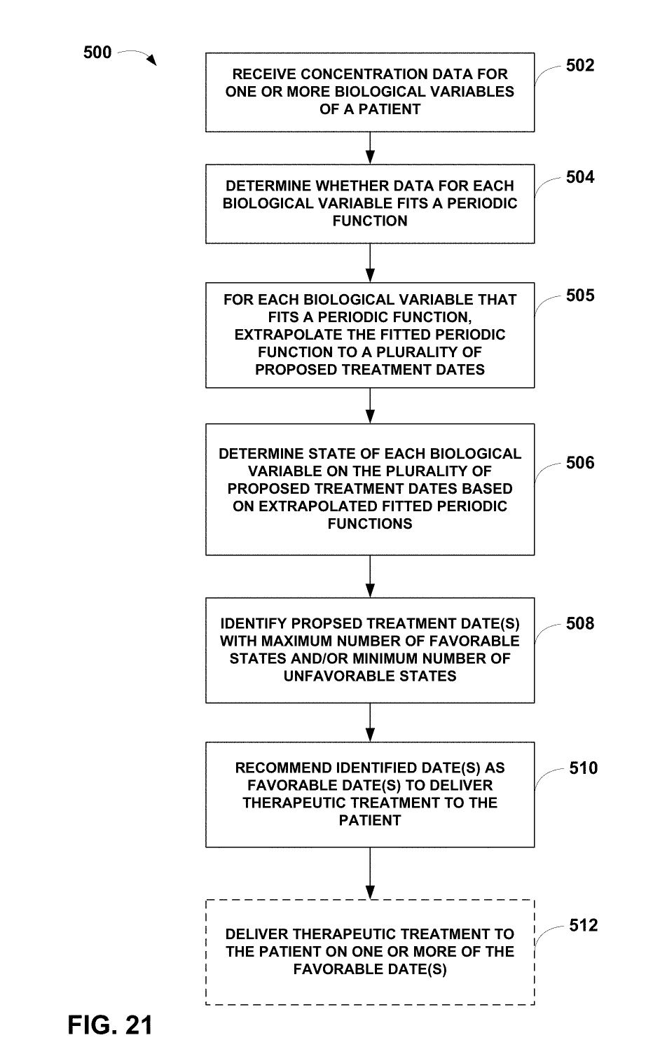

FIG. 21 is a flowchart illustrating an example process 500 by which a controller, such as controller 452 of system 450 shown in FIG. 20, may determine favorable treatment date(s) for delivery or administering of pharmacological or other therapeutic treatment, such as a chemotherapy treatment, to a patient. The controller may receive sets of time series data corresponding to concentrations of one or more biological variables in blood samples from the patient over an observed time period (502). The data may be obtained, for example, based on analysis of blood samples taken from the patient over a plurality of days or other observed time period. The biological variables may include, for example, one or more of IL-2; IL-10; IL-12p(70); G-CSF; IL-9; VEGF; CD206; IL-1ra; IL-13; IL-15; IL-17; CD3.4; CD3.8; CD4/294; CD11c/14; CD197/CD206; GRO; CD123.DR(DC2); CD11c/86; TGFa; IFNg; DR(hi); and/or any of the other biological variables listed in Table 1 or otherwise listed herein; and/or any other biological variables known to those of skill in the art. The biological variables measured in the samples of the patient may depend in part upon the disease or condition for which the patient is being treated, the type and/or frequency of the proposed treatment, or other factors. Thus, it shall be understood that any immune variable, biological variable, growth factor or counts of sub-populations of blood cells may also be included, in any combination, and the disclosure is not limited in this respect.

The controller (or other computing or processing system) analyzes the concentration data for each biological variable obtained from the patient to determine whether the data fits a period function (504). For example, the controller may determine whether the data fits a sinusoidal function. However, it shall be understood that the periodic function may be any periodic function, and that the disclosure is not limited in this respect. The controller may use any of a number of mathematical techniques known to those of skill in the art to detect a periodic pattern and fit a periodic function.

For each biological variable that fits a periodic function, the controller extrapolates the fitted periodic function to a plurality of proposed future treatment dates (505). The proposed treatment dates are future dates occurring subsequent to the observed time period during which the concentration data was collected. For example, the process may extrapolate the periodic function 5, 10, 15 or 20 days ahead of the observed time period. In this way, the process may analyze the data to identify which proposed treatment date in the near future is favorable for delivery of therapeutic treatment. In some examples, the process may extrapolate the periodic function for more or fewer days depending in part upon, for example, the periodicity of the periodic function, the number of data points obtained during the observed time period, the biological variables under analysis, the disease or condition for which the patient is being treated, the type and/or frequency of the proposed treatment, and other factors.

For each biological variable that fits a periodic function, the controller determines the state of the biological variable for each of the plurality of proposed treatment dates based on the extrapolated fitted periodic function (506). Again, in this example, the “state” of the biological variable refers to whether the concentration of the biological variable in the patient is greater than a predefined value on the proposed date of treatment (state=HIGH or UP), or less than the predefined value on the proposed date of treatment (state=LOW or DOWN). The purpose is to predict the state of the biological variable at future days on which treatment may be delivered.

In one example, states may be defined as (1) UP (greater than a threshold value associated with the biological variable) or (0) LOW (less than the threshold value associated with the biological variable). The controller may use any of a number of mathematical techniques to predict the states on the plurality of proposed future treatment dates. For example, the controller may analyze the time series data using various state predicting algorithms such as curve fitting by Levenberg-Marquardt (LM) algorithm, or LM augmented with extended Kalman filter or with unscented Kalman filter, expectation-maximization (EM) algorithms, machine learning applications, and any other method of detecting the state of the variable on future days. The states of the biological variables which are favorable for the treatment may be pre-defined based on previous clinical trial, such as shown and described above with respect to FIG. 19.

In one example, the threshold value dividing the two states (HIGH/UP and LOW/DOWN) is the median between the centroids of two clusters resulting from hierarchical clustering of all data points in a series. In another example, the threshold dividing the two states is an inter-cluster value defined as one-half the distance between the minimum values of the upper cluster and the maximum value of the lower cluster. A concentration value greater than the threshold dividing the two states is defined as the HIGH or UP state and a value less than the threshold dividing the two states is defined as the LOW or DOWN state. The process may compute a state for a given biological variable at any point in a time series (periodic function fitted to the data obtained during the observed time period) or for the extrapolated fitted periodic function for the proposed treatment dates.

The controller may determine one or more favorable treatment date(s) based on the states of the biological variables of the patient (508). In one example, the controller may identify one or more date(s) with a maximum number of favorable states. In another example, the controller may identify one or more date(s) with a maximum number of favorable states and/or a minimum number of unfavorable states. The date(s) when these conditions are met may be recommended as one or more favorable date(s) to deliver the pharmacological or other therapeutic treatment to a patient (510). In recommending favorable treatment date(s) (510) the process may also establish a treatment plan for the patient based on the one or more favorable date(s) to deliver the therapeutic treatment to the patient. For example, the controller may generate a report, display, or otherwise communicate a recommendation as to the one or more favorable date(s) to deliver the treatment to the patient and/or the treatment plan for the patient based on the favorable date(s) to deliver the treatment to the patient (510). In some examples, treatment may further be delivered to the patient on one or more of the identified favorable treatment date(s) (512).

In one example, fitting of the data to the sine curve may be done using a Levenberg-Marquart (LM) algorithm with an unscented Kalman filter (UKF) for noise reduction. The example algorithm predicts the state of the extrapolated fitted sine curve on a proposed future treatment date.

The LM least squared fitting algorithm for sinusoidal functions of the form f(t)=a+b*cos(c(t)+d) is sensitive to the initial parameters, especially the angular velocity parameter. If the initial parameter is too low, the algorithm converges to a solution with insufficient amplitude and a relatively high residual (doesn't go through or is not close to many of the points). If the initial (c) parameter is too high, the algorithm converges to a very rapidly oscillating sinusoid with a relatively low residual, but which does not make much biological sense.

Applying a Kalman filter to the LM process, may allow the system to account for noise in the process calculating optimal LM parameters for a sinusoid. That is to say, the system takes into account that the initial parameters have some noise associated with them and the algorithm combines this noisy guess with the noisy measurements at time (t) to give the true parameters for the sinusoid.

A more specific example is as follows:

Let the measured protein biological variable levels be the result of an underlying process with variables a, b, c and d (the parameters of the cosine function).

Let the underlying process be the LM algorithm with initial parameters a0, b0, c0, d0.

Let the function that takes us from the underlying state space to the measurement space be a+b*cos(c(t)+d).

As per the UKF algorithm, create sigma parameters for a0, b0, c0, and d0. For example, a Gaussian random variable centered around 0 with user specified variances for the a, b, c, and d parameters may be used.

Then for each actual measurement,

(1) Run all the sigma parameters through the LM algorithm. This results in a distribution of parameters (parameter distribution) which represent the predictions for the parameters when the sample is actually measured.

(2) Calculate the mean and variance of the parameter distribution generated in step 1.

(3) Create a predicted measurement distribution where each measurement in the distribution is calculated by using the function a+b*cos(c(t)+d) where a, b, c, and d are obtained from the prediction of the parameter distribution in the previous step, and t is the time for the current measurement.

(4) Calculate the mean and variance of the measurement distribution.

(5) Calculate the covariance of the measurement and parameter distribution.

(6) Calculate the Kalman factor K by dividing the mean variance of the measurement distribution by the covariance of the measurement and state distribution.

(7) Modify each state in the parameter distribution from step 1 such that Parameters x=Parameters x+K (actual measurement−mean Predicted Measurement (from step 4) and go back to step 1.

Those of skill in the art may recognize that the example given above is a modified version of a UKF. The example modifies that LM algorithm due to the use of the LM algorithm as a state function. The LM algorithm may also be written as a matrix root procedure.

In this example, the sigma parameters are not deterministic. They are generated by adding/subtracting the initial guess to/from a Gaussian Random Variable centered around 0 with a user specified variance.

In some examples, the sigma parameters are calculated once and are maintained in an array as they go through each iteration. This is different from the typical UKF implementation in which the average and variance of the parameters is calculated and is used to generate a new parameter distribution on the next iteration of the process. This may help to minimize the use of the Gaussian random variable since it may adversely affect the final solution depending on which sigma points are generated.

This may also help the accuracy of the LM algorithm. The LM algorithm is extremely sensitive to initial parameters when dealing with sinusoidal functions especially with respect to the angular momentum parameter. This is because if a sine function with an angular velocity of w (i.e. a+b*cos(w(t)+d) fits a set of points, then a sine function of integer multiples of w also fit the points. As a result, the parameter distribution may have a big variance and if an average is calculated and regenerated the parameter distribution to the solution may begin to stray very far from the initial guess. As a result, the example algorithm does not tune the predicted variance of the parameter states on each iteration, although it does tune the predicted state. After the last iteration, the example algorithm need not average the parameters in the parameter distribution for the final answer, it may choose the parameters that gives smallest residual.

The systems and methods described herein utilize spontaneously developed anti-tumor immunity by synchronizing delivery of therapy with dynamic (oscillating) changes of host immune homeostatic response. This synchronization may therapeutically deplete the elements of immune tolerance, and favor active anti-tumor immunity.

The role of the peripheral blood lymphocyte-to-monocyte ratio (LMR) may be an independent predictor of survival in many different hematological malignancies as well as solid tumors. In melanoma, for example, these two biomarkers may be independent predictors of relapse after surgical resection of stage III and IV disease.

For example, the lymphocyte-to-monocyte ratio (LMR) may be used for timing of therapy delivery as a surrogate of host immunity (i.e., CD3.4 lymphocytes) and immunosuppressive tumor microenvironment (i.e., CD14 tumor-associated monocytes co-expressing the dendritic marker CD11c). In melanoma, tumor-associated monocytes exhibit phenotypic and functional deficiencies that negatively affect their immune function. Monocytes are sensitive to the subgroup of methylating anticancer drugs such as TMZ due to a defect in base excision repair (BER) cellular mechanism which removes N-alkylating lesions from DNA. After exposure to methylating genotoxins, such as TMZ, the monocytic population is specifically depleted, whereas non-proliferating PBLCs and other blood compartments seem to be protected. Depletion of such cells, therefore, can be achieved by timed administration of TMZ just before or at the peak of concentration of CD11c.14 cells while non-proliferating host CD3.4 cells are at their low point.

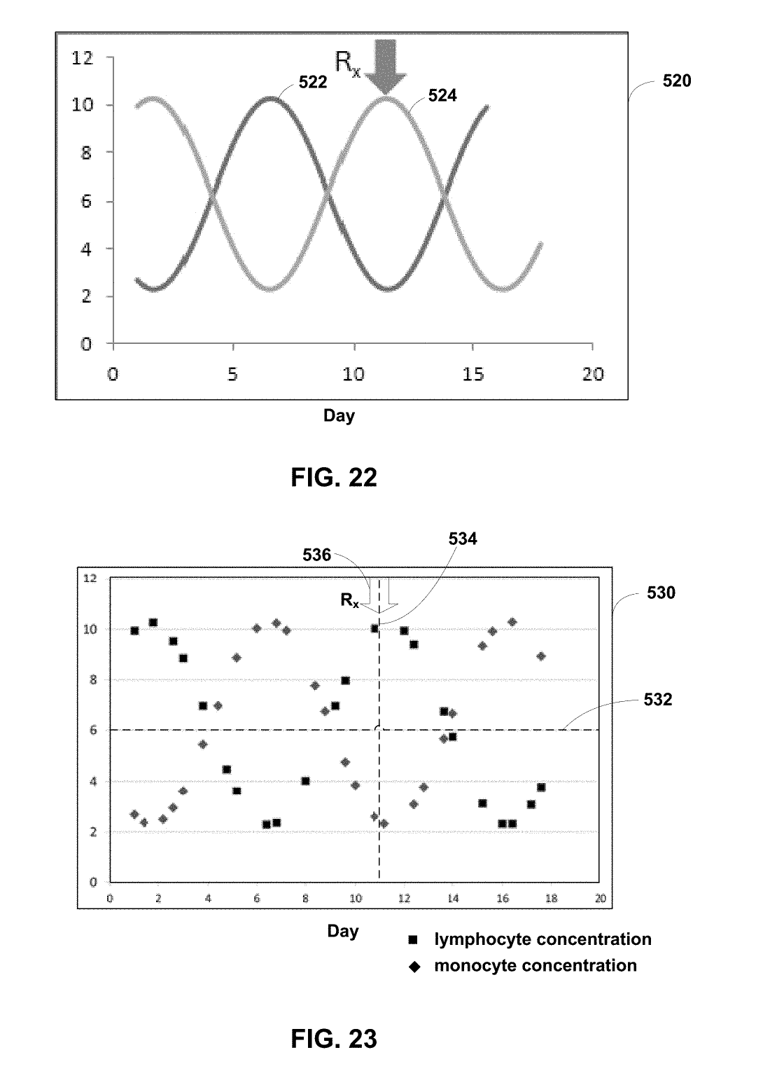

FIG. 22 is a graph 520 illustrating example lymphocyte (524) and monocyte (522) oscillations and identification of a favorable date, Rₓ, for delivery of therapeutic treatment to a patient based on a prognostic value of lymphocyte-to-monocyte ratio. In FIG. 22, the graph days 1-10 are the observed time period during which concentration data is obtained from blood samples of the patient. Days 11-20 are the dates for which the periodic function fitted to the data obtained during the observed time period (days 1-10 in this example) is extrapolated (days 11-20 in this example). The system and method described herein finds a day in the nearest future, when concentration of subsets of lymphocytes (CD3.4 cells) will be elevated (e.g., state=UP) and concentration of monocytes (CD11c14) will be diminished (e.g., state=DOWN). This day will be determined as a favorable date, Rₓ, for administration of therapy, such as chemotherapy in the melanoma example. In this example, the identified next favorable date, Rₓ, for chemotherapy delivery is day 11.

FIG. 23 is a graph 530 illustrating example lymphocyte (square-shaped data points) and monocyte (diamond-shaped data points) oscillations and identification of a favorable date, Rₓ, for delivery of therapeutic treatment to a patient. To define the state of a biological variable (lymphocytes and monocytes in this example), the algorithm takes time series data points (data corresponding to biological variable concentration taken over an observed time period) as an input and applies hierarchical clustering to this dataset. For example, two clusters may be generated using Euclidian distance as a measure of distance between data points. One-half of the distance between the minimum value of the upper cluster and the maximum value of the lower value may be returned as an inter-cluster value (ICV), indicated by reference numeral 532 in FIG. 23. A (concentration) value greater than the ICV is defined as the “UP” state (1) and a value less than the ICV is defined as the “DOWN” state (0). The ICV 532 in FIG. 23 is shown as being the same for both lymphocytes and monocytes; however, it shall be understood that each biological variable may have its own associated ICV or other threshold value. The process may compute a state for a given biological variable at any time point in a time series based on the periodic function fitted to the concentration data obtained during the observed time period. In the example of FIG. 23, the observed time period for which concentration data was obtained are Days 1-10.

The process may further predict the state of a biological variable for future proposed treatment dates based on the extrapolated fitted periodic function. In the example of FIG. 23, the extrapolated dates are Days 11-20. For each biological variable that fits a periodic function, the system extrapolates the fitted periodic function to a plurality of proposed future treatment dates. As described above, the proposed treatment dates are future dates occurring subsequent to the observed time period during which the concentration data was collected. For example, the process may extrapolate the periodic function 5, 10, 15 or 20 days ahead of the observed time period. In this way, the process may analyze the data to identify which proposed treatment date in the near future is favorable for delivery of therapeutic treatment. In some examples, the process may extrapolate the periodic function for more or fewer days depending in part upon, for example, the periodicity of the periodic function, the number of data points obtained during the observed time period, the biological variables under analysis, the disease or condition for which the patient is being treated, the type and/or frequency of the proposed treatment, and other factors.

The system further determines the state (UP or DOWN) on one or more proposed treatment dates based on the extrapolated periodic function. The system analyzes the states to identify a proposed treatment date when concentration of subsets of lymphocytes (e.g., CD3.4 cells indicated by the square-shaped data points) will be elevated (e.g., greater than a threshold value or state=UP) and concentration of monocytes (e.g., CD11c14, indicated by the diamond-shaped data points) will be diminished (e.g., less than a threshold value or state=DOWN). This day may be identified as a favorable date, Rₓ (Day 11 in this example), for administration of chemotherapy or other pharmacological treatment.

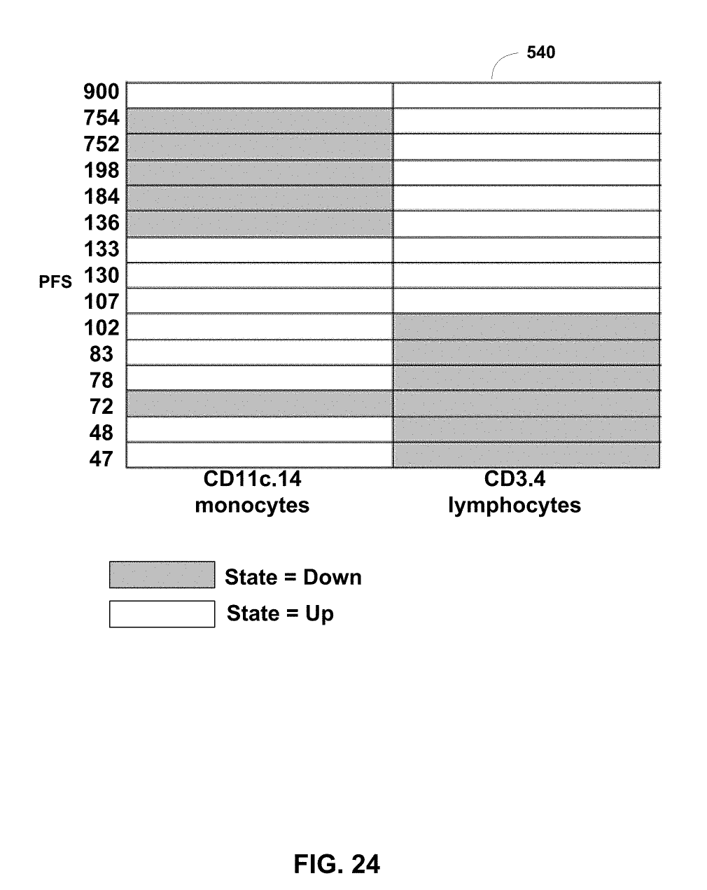

A retrospective Cox proportional hazards analysis of the data obtained in a clinical trial found statistically significant correlation between the state (UP or DOWN) of the CD3.4 (p-value<0.05) and CD11c14 (p-value<0.036) cell counts on the day of chemotherapy administration (TMZ) and progression-free survival (PFS) of the patients. FIG. 24 is a chart illustrating PFS and the state (UP or DOWN) for CDC11c.14 monocytes and CD3.4 lymphocytes. FIG. 24 shows that higher PFS is correlated with administration of chemotherapy on dates on which the CD11c.14 state is DOWN and the CD3.4 state is UP.

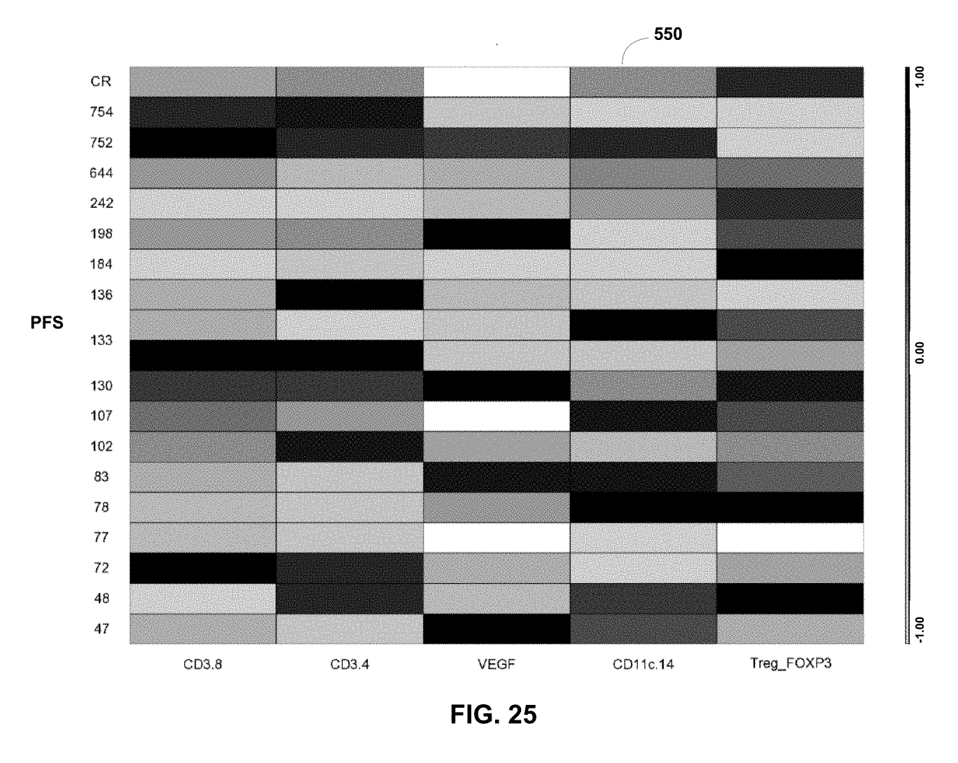

FIG. 25 shows the relative difference of concentration/counts (up—black or down—white) of 5 immune parameters (VEGF, Treg cells, CD11c.14, CD3.8 and CD3.4 cells) before and after timed delivery of therapeutic treatment as described herein as related to disease progression (PFS in days). CR stands for complete response. The color scale ranges from 1 (100% post-treatment increase relative to the pre-treatment value) to −1 (100% post-treatment decrease relative to the pre-treatment value).

Positive immune modulation with timed delivery of therapeutic treatment may also be obtained; that is, timed delivery of therapeutic treatment may help to modulate or maintain the favorable lymphocyte-to-monocyte ratio. The data in FIG. 25 shows positive immunomodulatory effect of timed TMZ administration in most patients with good disease control (PFS longer than 4 months), reflected in an increase in the percentage of CD8 effector T cells and CD4 T helper lymphocytes, along with a decrease in the “negative” immunological markers of response such as immunosuppressive CD11c.14 monocytes, Tregs, and the pro-angiogenic factor VEGF.

The lymphocyte-to-monocyte ratio (LMR) may be defined in several different ways. For example, the lymphocyte-to-monocyte ratio may include a ratio of the total predicted lymphocyte count on the proposed day of treatment to the total predicted monocyte concentration on the proposed day of treatment. As another example, the lymphocyte-to-monocyte ratio may include a ratio of the predicted percentage of lymphocytes (to the total white blood cell count) to the predicted percentage of monocytes (to the total white blood cell count).

The lymphocyte-to-monocyte ratio may be based on all known or measurable lymphocyte subtypes and/or all known or measurable monocyte subtypes. As another example, the lymphocyte-to-monocyte ratio may be based on a defined set of one or more lymphocyte subtypes and/or a defined set of one or more monocyte subtypes. The defined set of one or more lymphocyte subtypes and/or the defined set of one or more monocyte subtypes may be chosen based on an association with clinical outcome. As another example, the lymphocyte-to-monocyte ratio may apply a weighting function to the one or more lymphocyte subtypes in the defined set of one or more lymphocyte subtypes and/or the one or more monocyte subtypes in the defined set of one or more monocyte subtypes. As such, each of the one or more lymphocyte subtypes in the defined set of lymphocyte subtypes may have an associated weight, such that certain of the one or more lymphocyte subtypes in the set are weighted relatively more heavily than other of the one or more lymphocyte subtypes in the set. Similarly, each of the one or more monocyte subtypes may have a different associated weight, such that certain of the one or more lymphocyte subtypes in the set are weighted relatively more heavily than other of the one or more lymphocyte subtypes in the set. The weighting factor applied to each lymphocyte subtype and/or to each monocyte subtype may be based on the relative strength of an association for each lymphocyte subtype and/or each monocyte subtype to clinical outcome.

As another example, the lymphocyte-to-monocyte ratio may be based on the predicted state (e.g., UP or DOWN) of the one or more lymphocyte subtypes in the defined set of lymphocyte subtypes on the proposed date of treatment, and/or the predicted state (e.g., UP or DOWN) of the one or more monocyte subtypes in the defined set of monocyte subtypes on the proposed date of treatment. For example, the lymphocyte-to-monocyte ratio may be based on the number of lymphocyte subtypes in the UP (favorable) state and/or the number of monocyte subtypes in the DOWN (favorable) state. In one example, the proposed treatment date may be chosen as the date on which the maximum number of the one or more lymphocyte subtypes are predicted to be in an UP state and a maximum number of the one or more monocyte subtypes are predicted to be in a LOW state. In another example, weights may be applied to the predicted states of the one or more lymphocyte subtypes in the defined set of lymphocyte subtypes, and/or to the predicted states of the one or more monocyte subtypes in the defined set of monocyte subtypes, such that certain of the one or more lymphocyte subtypes are weighted relatively more heavily than other of the one or more lymphocyte subtypes in the defined set of lymphocyte subtypes, and/or certain of the one or more monocyte subtypes are weighted relatively more heavily than other of the one or more monocyte subtypes in the defined set of monocyte subtypes.

Further, the particular lymphocyte subtypes and/or monocyte subtypes used to define or calculate the lymphocyte-to-monocyte ratio may be different for different types of cancers, for different types of therapy, or may be specific to each patient.

In this manner, in one example, a method of cancer (or other disease) treatment may include administering chemotherapy treatment to a patient on a favorable treatment date identified based on a predicted lymphocyte-to-monocyte ratio in the blood of the patient on the favorable treatment date. In another example, a method of cancer treatment may include administering chemotherapy treatment to a patient on a favorable treatment date identified based on a predicted state of at least one lymphocyte subtype in the blood of the patient on the favorable treatment date, and on a predicted state of at least one monocyte subtype in the blood of the patient on the favorable treatment date.

In the following study, the dynamics in the immune system of patients with stage IV melanoma was examined by performing serial concentration measurements of cytokines and immune cell sub-types in peripheral blood. We then analyzed outcomes of chemotherapy administration as related to LMR in the blood on the day of treatment initiation. The results showed that progression-free survival is significantly improved in patients who received chemotherapy on the day when LMR was elevated.

Temozolomide (TMZ) is an oral alkylating chemotherapeutic agent which is administered for metastatic melanoma as 200 mg/m2 daily dose over 5 days with cycles repeated every 28 days. These standard TMZ doses result in serum concentrations of up to 50 μM, which have been shown in preclinical models to cause dose-dependent apoptosis preferentially in proliferating monocytes which are depleted of MGMT (O6-methylguanine-DNA methyltransferase), a repair enzyme. Kinetic studies show that TMZ induced DNA strand breakage and apoptosis of susceptible immune cells is detectable at 4 hours after oral administration. However, therapeutical doses of TMZ did not impair non proliferating peripheral blood lymphocytes or the function of either CD8+ effector T-cells and dendritic cells (DCs) in vitro. One may therefore utilize the immunomodulatory properties of classical cytotoxic agents by administering these drugs in such a way that will stimulate the intrinsic anticancer immune response. Timed administration of this agent may selectively suppress immunosuppressive elements (such as tumor-associated monocytes) when cytotoxic chemotherapy is timed with mitosis, a time when they are particularly vulnerable to the alkylating agent. When monocytes undergo rapid clonal expansion they seem to do so in a synchronous logarithmic fashion and thus are all vulnerable to anti-mitotic agents. Once these immune suppressive cells have been removed by therapeutic intervention, the immune response is “unblocked” leading to restoration of immune balance. Oscillations of LMR may be used therapeutically to identify the correct time point in the immune cycle to deliver cytotoxic chemotherapy, which would be when LMR is elevated and immune suppressive monocytes are starting to proliferate to induce the next down-swing in the anti-cancer immune response. Although TMZ would theoretically inhibit desirable effector T cells as well, timed administration of this agent may selectively suppress proliferating monocytes while effector T cells proliferate and become activated before administration, and are as such replenished in MGMT and less susceptible to the effects of TMZ chemotherapy. The administration may be oral or intravenous. Moreover, the kinetics of MGMT changes in proliferating lymphocytes and monocytes and the induction of apoptosis by TMZ over days indicate that timed delivery of this agent in the context of infradian immune dynamics may achieve a controlled immune depletion and generate positive immunomodulatory effects in addition to its direct cytotoxicity.

Eligible patients had unresectable, histologically confirmed stage IV disease, age over 18 years, measurable disease as defined by the Response Evaluation Criteria in Solid Tumors (RECIST), Eastern Cooperative Oncology Group (ECOG) performance status of 0-2, and life expectancy≥3 months (See Table 10). Both previously untreated patients and patients who have had prior therapy for their metastatic disease (excluding prior exposure to TMZ) were eligible. All patients provided signed informed written consent, and the study was approved by the Mayo Clinic Rochester Institutional Review Board. The TMZ dose was 150 mg/m2 on days 1-5 of cycle 1 and was increased to 200 mg/m2 on days 1-5 each month for all subsequent cycles if well tolerated. Patients were treated every 28 days until progression, unacceptable toxicity or patient refusal. Prior to initiation of first chemotherapy cycle, eligible patients underwent serial daily peripheral blood testing for 10 days of 69 immune biomarkers (42 cytokines, and 27 immune cell subtypes) per time point/patient. At the end of the sample collections, all data was jointly analyzed and a day for individualized TMZ delivery was calculated according to the proposed model (below). Patients received their therapy during the time, predicted by the model. All patients were followed for clinical outcomes.

Time-dependent profiles of blood concentration of IL-12p70, CD197/CD206, IL-1ra were built based on a series of pre-treatment measurements. The data was fitted to a sine curve and the extrapolated for a period of 10 days. Chemotherapy was initiated on the day when the extrapolated value of the combination of these immune parameters was at maximum. The results are shown in Table 11.

TABLE 10
Contingency table used for Fisher exact test.

| | PFS <4 months | PFS >4 months | Total |
|---|---|---|---|
| LMR >1 | 3 | 6 | 9 |
| LMR <1 | 7 | 1 | 8 |
| TOTAL | 10 | 7 | 17 |

TABLE 11
Patients' demographics and dates on the study MC1076

| | | | Enrollment | Started | Progression | |
|---|---|---|---|---|---|---|
| Patient ID | Sex | Age | date | TMZ | date | PFS |
| MC1076_1 | M | 76 | Apr. 11, 2011 | Apr. 28, 2011 | Jun. 27, 2011 | 77 |
| MC1076_2 | M | 70 | May 17, 2011 | Jun. 2, 2011 | Sep. 27, 2011 | 133 |
| MC1076_3 | M | 63 | Jun. 6, 2011 | Jun. 28, 2011 | Dec. 21, 2011 | 198 |
| MC1076_4 | M | 71 | Jun. 23, 2011 | Jul. 6, 2011 | CR | CR |
| MC1076_5 | M | 68 | Sep. 16, 2011 | Oct. 8, 2011 | TBD | 644 |
| MC1076_6 | M | 75 | Nov. 4, 2011 | Nov. 23, 2011 | Jul. 3, 2012 | 242 |
| MC1076_7 | F | 69 | Dec. 5, 2011 | Dec. 21, 2011 | Aug. 7, 2012 | 184 |
| MC1076_8 | M | 65 | Dec. 8, 2011 | Dec. 28, 2011 | Jan. 24, 2012 | 47 |
| MC1076_9 | M | 75 | Dec. 9, 2011 | Dec. 27, 2011 | Mar. 1, 2012 | 83 |
| MC1076_10 | F | 36 | Jan. 6, 2012 | Jan. 26, 2012 | Nov. 2, 2012 | 301 |
| MC1076_11 | F | 77 | Aug. 20, 2012 | Sep. 9, 2012 | Jan. 3, 2013 | 136 |
| MC1076_12 | F | 83 | Sep. 10, 2012 | Sep. 27, 2012 | CR | CR |
| MC1076_13 | F | 39 | Sep. 12, 2012 | Oct. 11, 2012 | Oct. 4, 2014 | 752 |
| MC1076_14 | M | 62 | Sep. 20, 2012 | Oct. 11, 2012 | Dec. 31, 2012 | 102 |
| MC1076_15 | M | 81 | Oct. 10, 2012 | Oct. 30, 2012 | Dec. 27, 2012 | 78 |
| MC1076_16 | F | 67 | Oct. 16, 2012 | Nov. 7, 2012 | Nov. 24, 2012 | 39 |
| MC1076_17 | M | 70 | Oct. 17, 2012 | Nov. 7, 2012 | Feb. 1, 2013 | 107 |
| MC1076_18 | F | 62 | Nov. 6, 2012 | Dec. 3, 2012 | Jan. 17, 2013 | 72 |
| MC1076_19 | F | 84 | Nov. 23, 2012 | Dec. 12, 2012 | Jan. 15, 2013 | 53 |
| MC107_20 | F | 60 | Jan. 2, 2013 | Jan. 23, 2013 | Feb. 19, 2013 | 48 |
| MC1076_21 | F | 76 | Jan. 7, 2013 | Jan. 27, 2013 | Mar. 28, 2013 | 80 |
| MC107622 | M | 63 | Jan. 15, 2013 | Feb. 1, 2013 | Mar. 28, 2013 | 72 |
| MC1076_23 | M | 72 | Feb. 4, 2013 | Feb. 20, 2013 | Apr. 18, 2013 | 73 |
| MC1076_24 | M | 60 | Feb. 22, 2013 | Mar. 13, 2013 | Jul. 2, 2013 | 130 |

The same measurements as in melanoma patients were performed in 3 healthy individuals, as shown in Table 12.

TABLE 12
Demographics for healthy individuals

| Patient ID | Sex | Age |
|---|---|---|
| H_1 | F | 51 |
| H_2 | F | 56 |
| H_3 | F | 57 |

In order to study the global behavior of the anti-tumor immune response in metastatic melanoma, peripheral blood samples obtained prior to initiation of TMZ therapy were subsequently analyzed for plasma concentration of 42 different cytokines and 22 immune cell subsets (described below). All biospecimens were collected, processed, and stored following established and validated standard operating procedures in our laboratory 4. To reduce inter-assay variability, all assays were batch-analyzed after study completion. All blood samples were collected at approximately the same time of day (between 0800 h and 01000 h) in order to minimize the contribution of circadian variation to the fluctuation of immune parameters.

Peripheral blood mononuclear cell (PBMC) immunophenotyping for immune cell subsets. Blood was separated into platelet poor plasma and PBMC using a density gradient (Ficol-hypaque, Amersham, Uppsala, Sweden). Plasma samples were stored at −70° C., and PBMC were stored in liquid nitrogen. Immunophenotyping of PBMC was performed by flow cytometry using FITC- and PE-conjugated antibodies to CD3, CD4, CD8, CD16, CD56, CD62L, CD69, TIM3 (T-cell immunoglobulin domain and mucin domain 3), CD294, HLA-DR, CD11c, CD123, CD14, CD197, CD206, and B7-H1 (Becton-Dickinson, Franklin Lakes, N.J.). In addition, intracellular staining for FoxP3 (BioLegend, San Diego, Calif.) was performed according to the manufacturer's instructions. Data were processed using Cellquest® software (Becton-Dickinson, Franklin Lakes, N.J.). PBMC bio-specimens were analyzed for the frequencies of T cells (CD3+), T helper cells (CD3+4+), CTL (CD3+8+), natural killer cells (NK, CD16+56+), T helper 1 (Th1) cells (CD4+TIM3+), Th2 cells (CD4+294+), T regulatory cells (Treg, CD4+25+FoxP3+), type 1 dendritic cells (DC1, CD11c+HLA-DR+), type 2 dendritic cells (DC2, CD123+HLA-DR+), type 1 macrophages (M1, CD14+197+), type 2 macrophages (M2, CD14+206+) and for the activation status of these cell types. In order to access the Th1/Th2 balance we stained PBMC with anti-human CD4, CD294, and TIM-3. The stained cells were analyzed on the LSRII (Becton Dickinson Franklin Lakes, N.J.). The CD4 positive population was gated and the percent of CD4 cells positive for either CD294 or TIM-3 was determined. Our preliminary data suggests that CD4/CD294 positive Th2 cells exclusively produce IL-4 and not IFN-γ upon PMA and ionomycin stimulation (data not shown). Conversely, CD4/TIM-3 positive Th1 cells exclusively produce IFN-γ and not IL-4 following the same in vitro stimulation. Enumeration of Treg was performed using intracellular staining for FoxP3 of CD4/25 positive lymphocytes.

Plasma cytokine profiling. Protein levels for 42 cytokines, chemokines, and growth factors, including interleukin 1-alpha IL-1a, IL-1b IL-1ra, IL-2, IL-3, IL-4, IL-5, IL-6, IL-7, IL-8, IL-9, IL-10, IL-12(p40) IL-12(p70), IL-13, IL-15, IL-17A, basic fibroblast growth factor (FGF-2), Eotaxin, granulocyte colony-stimulating factor (G-CSF), granulocyte-macrophage colony-stimulating factor (GM-CSF), interferon (IFN-gamma), IFNa2, 10 kDa interferon-gamma-induced protein (IP-10), macrophage chemoattractant protein 1 (MCP-1), MCP-3, migration inhibitory protein 1 (MIP-1a), MIP-1b platelet-derived growth factor (PDGF-AA), PDGF-AB/BB, Regulated upon Activation Normal T-cell Expressed and Secreted (RANTES), tumor necrosis factor alpha (TNF-α), TNF-b, epithelial growth factor (EGF), Flt3 ligand, fractalkine, growth related oncogene (GRO), monocyte derived chemokine (MDC), soluble CD40L, transforming growth factor-alpha (TGF-a) vascular endothelial growth factor (VEGF) were measured using the Millipore human 42-plex cytokine panel (Cat # HCYMAG-60K-PX42, Millipore, Billerica, Mass.) as per manufacturer's instructions. Transforming growth factor beta (TGF-β1) was measured separately using a quantitative ELISA test and CRP concentration was measured in real time using a clinical laboratory test 20. All plasma cytokine measurements were performed in duplicates. Normal values for plasma cytokine concentrations were generated by analyzing 30 plasma samples from healthy donors (blood donors at the Mayo Clinic Dept. of Transfusion Medicine). A set of three normal plasma samples (standards) was run alongside all batches of plasma analysis in this study. If the cytokine concentrations of the “standard” samples differed by more than 20%, results were rejected and the plasma samples re-analyzed.

The MC1076 study was designed to assess the anti-tumor activity of timed administration of TMZ. A two-stage phase II clinical trial design will be used to test the hypothesis that the 4 month PFS rate is at most 45% against the alternative hypothesis that the 4 month PFS rate is at least 65%. Progression-free survival was defined as the time from registration to documentation of disease progression or death without disease progression documented.

Serial concentration measurements of peripheral blood biomarkers were analyzed using custom developed software to construct time-dependent profiles of plasma cytokine/immune cell counts. We applied a two-step algorithm to detect oscillatory patterns in these profiles in study MC1076. At the first step we used an algorithm for assessment of periodicity in short data sets. We considered 3 published algorithms (autocorrelation, autocorrelation enhanced with Fisher's g-statistic assessment of significance, and coherence function analysis (CFA)) for assessment of periodicity in short data sets. We performed a computer simulation study aimed to compare false discovery rates (FDR) of these algorithms. The study has demonstrated that CFA produced the lowest FDR. Therefore, in our further analyses CFA was used to detect oscillatory patterns in time-dependent concentration profiles. At the next step, data series that were defined as oscillatory by the CFA, were fitted to a cosine curve using Levenberg-Marquardt algorithm. The R² value was used as a “goodness-of-fit” criterion. A profile was defined as oscillatory if the R² value was greater than the 75^{th} percentile of the distribution of all R² for all cytokines across all patients. Partek software (Partek Inc. St Louis, Mo.) was used for data formatting, editing and visualization.

Analysis of infradian (multi-day) dynamics in systemic immunity of patients with metastatic melanoma. Based on data, both published and from our lab, we hypothesized that the anti-tumor systemic immune response in patients with cancer is not a static but a dynamic event. In order to study the global temporal behavior of systemic anti-tumor immunity, we conducted a phase II clinical trial (MC1076; NCT01328535). In this study, patients with metastatic melanoma underwent a period of immunological monitoring consisting of 10 daily PB collections (10 mL) over 12 days prior to initiation of therapy with TMZ. There was no blood collection on weekend days. At the end of the sample collection period, samples were batched analyzed (to minimize inter-assay variability for a given patient) for measurement of dynamic changes of plasma cytokines and immune cell subsets using established, standardized, and Good Laboratory Practice (GLP)-validated methodology in our laboratory.³⁵ Time-dependent profiles of immune variables (cytokines and immune cell subsets) were then analyzed and an optimal day for initiation of TMZ therapy was prospectively computed by the algorithm described below. The overall goal of this study was to assess if immune system of melanoma patients undergoes periods of up- and down-regulation and, if so, how this dynamic is related to clinical outcome. The study aimed to validate the hypothesis that the time when several regulators of immune system have elevated blood levels is optimal for the administration of chemotherapy. The list of these regulators included IL-12p70, IL-10, VEGF, G-CSF, IL-10, IL-12p70, IL-1ra, GM-CSF, IL-13, IL-17, IL-9, G-CSF, IL-15, IL-17, GRO, IL-12p40, IL-2, IFNg, IL-1a, IP-10, IL-7, IL-4, IL-6, IL-1ra.

Retrospective analysis of lymphocyte-to-monocyte ratio in patients from MC1076 study. Of the multitude of measured immune parameters in the study MC1076, the ratio of the counts of CD3+/4+ cells to the counts of CD11c+/14+ cells was found to be statistically significantly related to clinical outcome. Since the MC1076 study was not designed specifically to assess the relevance of the lymphocyte-to-monocyte ratio during the pre-treatment period and particularly on the day of therapy administration to clinical outcome, we performed a retrospective analysis of the data collected in this study. The analysis aimed to assess i) whether lymphocyte-to-monocyte ratio is steady (for example is steadily greater than 1) or it is variable over time; ii) whether there exists a correlation between clinical outcome assessed by progression-free survival time (PFS) and the lymphocyte-to-monocyte ratio on the day of chemotherapy administration. To obtain this ratio we used the concentration of CD3+CD4+ lymphocytes as a marker of host immunity and concentration of CD11c+CD14+ as an indicator of tumor-associated monocytes (CD14 tumor-associated monocytes co-expressing the dendritic marker CD11c). The LMR was calculated as a ratio of concentrations of CD3+4+ lymphocytes to CD11c+CD14+ monocytes for each day during the 12-day pre-treatment period and for the day of therapy initiation. Blood samples on the first day of therapy were collected from only 17 patients out of 24. Therefore, we have no LMR values for 7 patients on the day of therapy initiation.

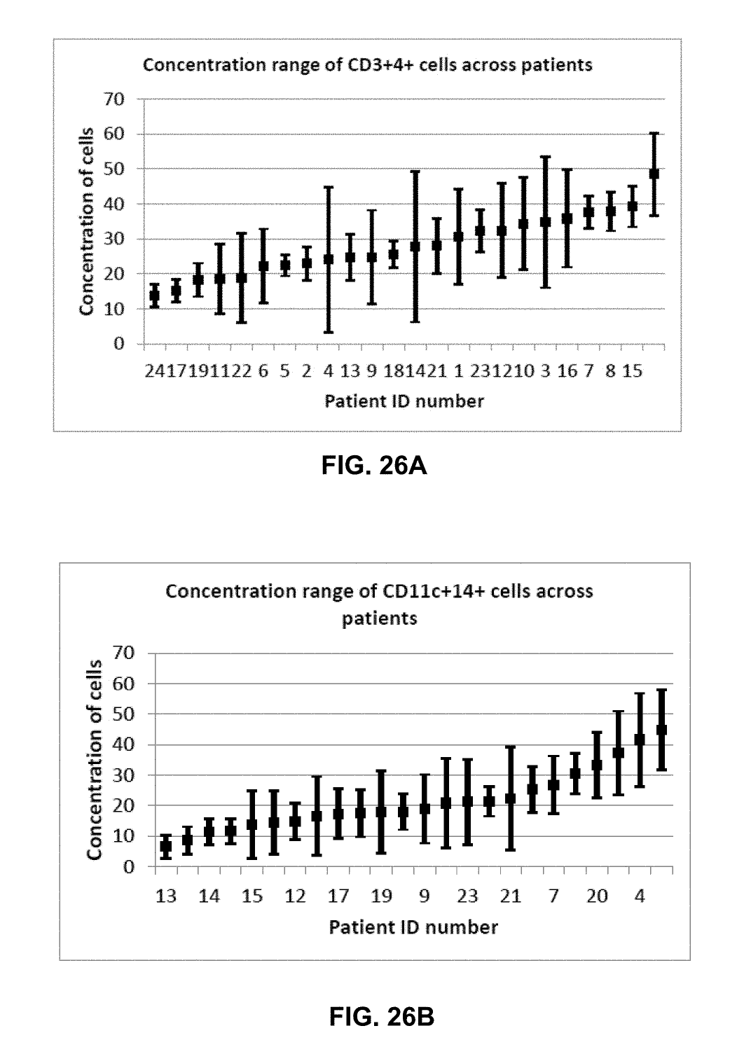

The data (Table 11) showed that the range of concentrations of either type of cells varies across patients (See FIGS. 26A and 26B). There is also a significant trend (correlation coefficient=0.94, N=24) of correlation between the average frequencies of the two cell types.

FIGS. 26A and 26B show blood concentration range of CD3+4+ cells (FIG. 26A) and CD11c+14+ cells (FIG. 26B). Vertical bars represent minimum to maximum interval of concentrations for each patient. The tick in the middle of the bar represents mean concentration. The data shows that the range of concentrations of either type of cells varies across patients (FIGS. 26A and 26B).

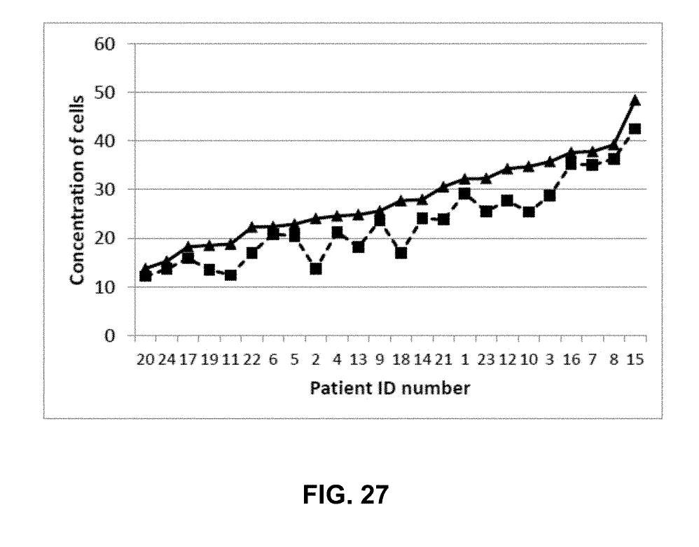

FIG. 27 shows mean concentration of CD3+4+(triangles, solid line) and CD11c+14+(squares, dashed line) cells for each patient sorted in ascending order of CD3+4+ concentration. Correlation coefficient between concentration values is 0.94. The data shown in FIG. 27 indicates that there is also a significant trend (correlation coefficient=0.94, N=24) of correlation between the average frequencies of the two cell types.

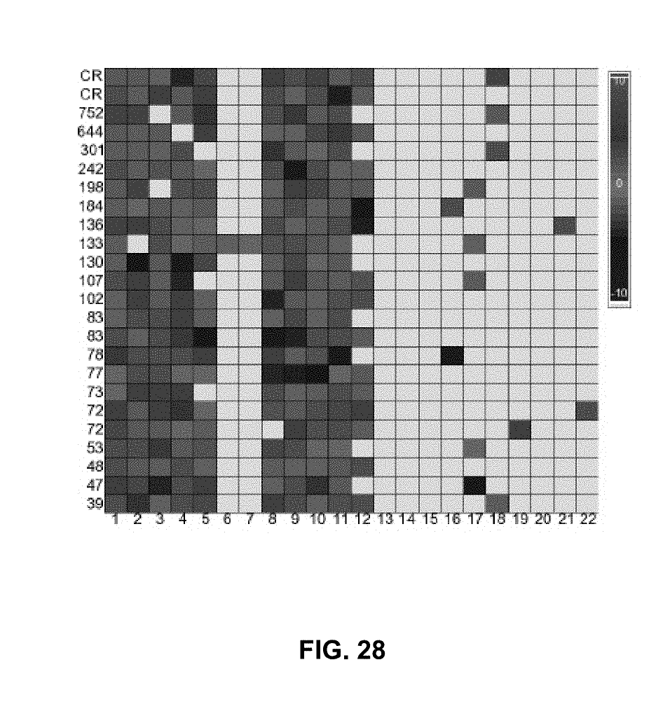

The dynamic of lymphocyte-to-monocyte ratio in melanoma patients. Because the range of cell concentration varies across patients, we normalized concentration values by scaling them between 0 and 1 by the formula: (Cₑₓₚ−Cₘᵢₙ)/(Cₘₐₓ−Cₘᵢₙ), where Cₑₓₚ is experimental concentration, Cₘᵢₙ is the minimum concentration and Cₘₐₓ is the maximum concentration for individual patient including the concentration on the first day of therapy. This numerical transformation was necessary in order to adequately compare LMR across patients and assess its correlation with clinical outcome. Since LMR is a ratio of two concentration values, scaling these values from 0 to 1 in each patient enables to directly compare LMR between patients. The dynamic of lymphocyte-to-monocyte ratio for 24 patients is represented as a heat map (e.g., FIG. 28). Blood concentration of monocytes and lymphocytes on the day of treatment initiation was measured for 17 patients, and was not measured for the remaining 7 patients.

FIG. 28 shows an example dynamic of lymphocyte-to-monocyte ratio in melanoma patients. To adequately represent the range of ratios where lymphocytes are prevalent over monocytes (LMR>1) (or, in the case of states, lymphocytes are state=UP and monocytes are state=DOWN) and the range of ratios where monocytes are prevalent over lymphocytes (0<LMR<1) (or, in the case of states, lymphocytes are state=DOWN and monocytes are state=UP), LMR is represented as fold change (FC) by the formula {FC=LMR when LMR>=1; FC=−1/LMR when 0<LMR<1}. Turquoise color represents the whole time span of the study. The dynamic of FC values for each patient is displayed in rows of colored blocks. Progression-free survival of each patient is shown on the left side of the map. Blocks colored with shades of red represent FC greater than 1 (LMR>1), shades of blue represent FC less than 0 (0<LMR<1). Gray corresponds to 0. Empty blocks denote missing data. Numbers along the X-axis represent day count after the enrollment on the study. Blocks in the time interval from day 16 to 29 show FC on the day of treatment. Blood concentration of monocytes and lymphocytes on the day of treatment initiation was measured for 17 patients, and was not measured for the remaining 7 patients. CR denotes complete response.

As demonstrated in FIG. 28, LMR is not a steady value, but this ratio may vary as much as 41 fold, from 19.45 (prevalence of lymphocytes over monocytes) to 0.47 (prevalence of monocytes over lymphocytes) in the same patient over a period of 12 days. One can observe in the heat map that in several patients high LMR (red blocks) alternates with low LMR values (blue blocks) (patients with PFS 644, 301, 198, 136, 102, 107, 83, 77, 72 (patients' numbers: 5, 10, 3, 11, 14, 17, 9, 1, 22).

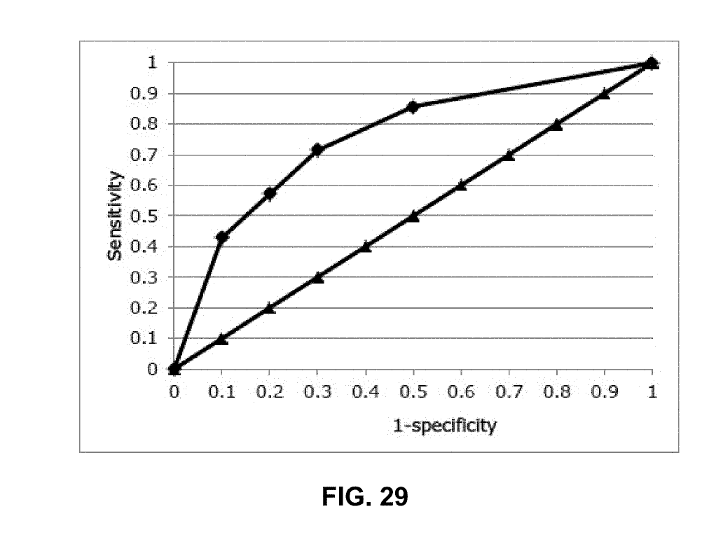

To test the hypothesis that LMR on the day of treatment is related to clinical outcome, we first performed the receiver operating characteristic (ROC) analysis. FIG. 29 shows receiver operating characteristic curve for lymphocyte-to-monocyte ratio (LMR) on the day of initiation of treatment with temozolomide. The area under the curve (AUC) is 0.78. Straight line with triangle markers represents theoretical curve for random distribution of measured values (LMR). The results showed that i) patients with early progression (PFS<4 months) can be grouped separately from patients with extended progression (PFS>4 months) based on the LMR value on the day of treatment. The area under the ROC curve was 0.78. ii) In this example, the optimal cut-off value of LMR that separates the patients with early progression from patients with extended progression is 1. Based on these results we divided patients into two groups—those in whom LMR on the day of TMZ initiation was greater than 1 and those in whom LMR on the day of TMZ initiation was less than 1. We then grouped patients into two groups by clinical outcome—PFS greater than 4 months and PFS less than 4 months. Using these groupings, we constructed 2×2 matrix (Table 1) and performed Fisher exact test. The two-tailed p-value of the test was (0.049) indicating that the association between LMR and clinical outcome (PFS) is statistically significant.

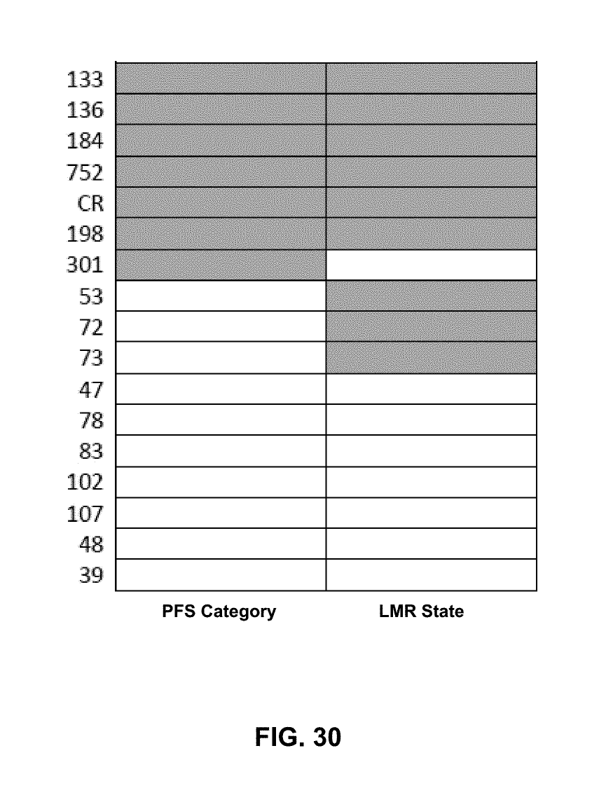

The association of PFS with LMR state (LMR>1 or LMR<1) is represented as a heat map in FIG. 30. FIG. 30 shows association between PFS and LMR state. Patients were divided by clinical outcome into two groups—those who progressed in less than 4 months since the enrollment into the study (PFS<4, 120 days) and those who progressed after 4 months or more (PFS>4). By the state of LMR on the day of TMZ initiation patients were classified also into two groups—those with LMR<1 (blue blocks) and those with LMR>=1 (red blocks). The numbers on the left of the plot represent PFS in days. CR stands for complete response. There were four patients in whom the PFS-LMR association was reversed. Two of these patients (corresponding PFS numbers are 301 and 53) had ocular, rather than cutaneous melanoma.

The association between PFS and blood concentration of CD3+4+ and CD11c+14+ cells. Next, we validated if blood concentration of CD3+4+ and CD11c+14+ cells at the time of initiation of TMZ therapy correlates with PFS. In this analysis we used the same analytical methods as for the analysis of LMR. First, we constructed ROC curves to find the optimum cut-off concentration values. Using these cut-off values to separate patients in groups based on the concentration of CD3+4+ cells or CD11c+14+ cells, we performed Fisher exact test. The two-tailed p-value for CD3+4+ cells was 0.36 and for CD11c+14+ cells the p-value was 0.59 which indicates that blood concentration of neither of these cell types correlates with PFS.

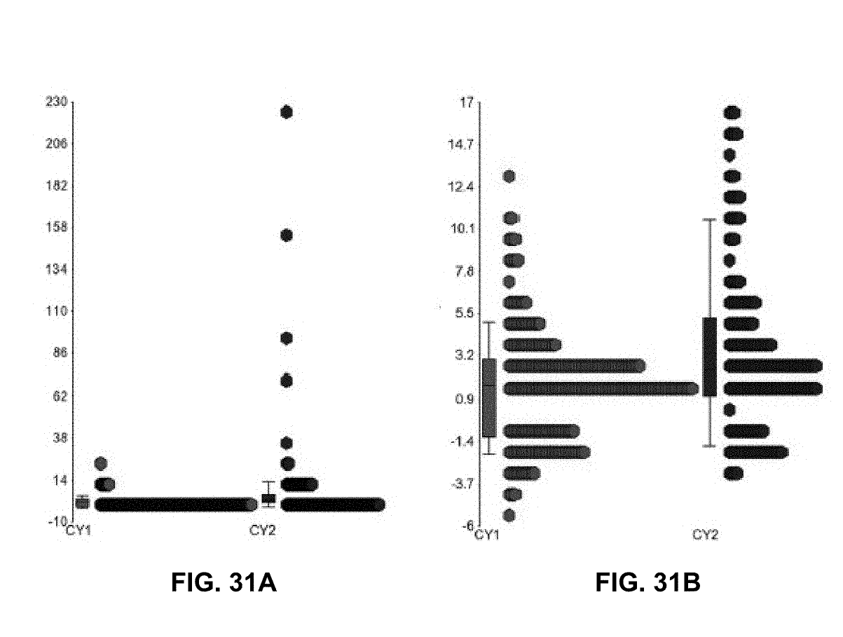

The range of values and oscillatory pattern of LMR in healthy individuals. The maximum range of LMR values that was observed in a healthy individual was 4.4 fold (from 6.42 (prevalence of lymphocytes over monocytes) to 1.5 (prevalence of monocytes over lymphocytes) which is 10 times less than in melanoma patients. We then applied Wilcoxon rank-sums test to assess the difference of the distributions of LMR values between three groups—1) healthy individuals 2) melanoma patients before treatment 3) melanoma patients after treatment (FIGS. 31A and 31B). FIGS. 31A and 31B show distribution of FC values (for explanation please see legend for FIG. 28) in melanoma patients before (CY1) and after (CY2) treatment. FIG. 31A shows the overall distribution and FIG. 31B shows the distribution in the range from −6 to 17 (to enhance resolution). Color bars represent 25^{th} (bottom of the bar), 50^{th} (middle) and 75^{th} (top) percentiles and whiskers represent outliers.

The Wilcoxon test indicated that the differences between groups 1 and 2 and between groups 2 and 3 were significant (p-values=0.0004 and 0.0001, respectively). However, the difference between groups 1 (healthy) and 3 (melanoma patients after treatment) was not significant (p-value=0.49).

This preliminary data demonstrated the presence of temporal kinetics of lymphocytes and monocytes in patients with advanced cancer, which appear to oscillate between states of “up” and “down” regulation. As a result, the LMR in many patients has an oscillatory pattern. These oscillatory changes in the lymphocyte-to-monocyte ratio may explain some of rare but significant benefits of cancer immunotherapy in humans (treatment given at the specific time). Retrospectively, we analyzed our data to find that LMR on the day of treatment significantly correlated to PFS; therefore, LMR may be useful tool in determining the best day to administer chemotherapy.

Here we presented results that suggest that in stage IV melanoma patients LMR varies over time. Our data further suggest that efficacy of cytotoxic chemotherapy is improved when the treatment is administered at the time when LMR is elevated. For example, our results demonstrated that the ratio of CD3+4+/CD11c+14+ cells is a better marker of treatment efficacy than blood concentration of either of these cell types by themselves. Based on these results, it may be reasonable to hypothesize that the measurements of sub-types of immune cells and their ratios on the day of therapy may yield more precise marker of treatment efficacy. It is also plausible that each cancer type may have different pertinent markers, which could also vary with the type of treatment (i.e., cytotoxic vs immunotherapy).

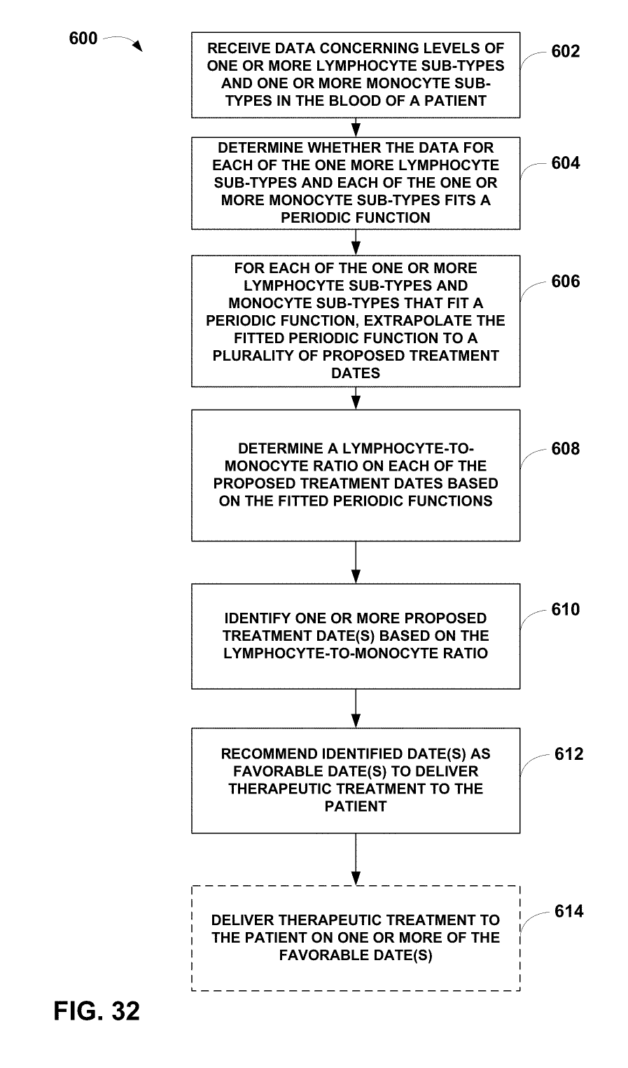

FIG. 32 is a flowchart illustrating another example process (600) by which a controller may determine favorable treatment date(s) for delivery of chemotherapy treatment (or other type of therapeutic or pharmacological treatment) to a patient. The controller may receive sets of time series data corresponding to levels (concentrations, absolute counts, etc.) of one or more lymphocytes and one or more monocytes in blood samples from the patient over an observed time period (602). The data may be obtained, for example, based on analysis of blood samples taken from the patient over a plurality of days or other observed time period. The particular subset of lymphocytes and/or monocytes measured in the samples of the patient may depend in part upon the disease or condition for which the patient is being treated, the type and/or frequency of the proposed treatment, or other factors. Thus, it shall be understood that any lymphocytes, monocytes, or other type of cell may also be included, in any combination, and the disclosure is not limited in this respect.

The controller (or other computing or processing system) analyzes the data for each of the one or more lymphocytes and each of the one or more monocytes obtained from the patient and determines whether the data fits a period function (604). For example, the controller may determine whether the data fits a sinusoidal function. However, it shall be understood that the periodic function may be any periodic function, and that the disclosure is not limited in this respect. The controller may use any of a number of mathematical techniques known to those of skill in the art to detect a periodic pattern and fit a periodic function.

For each of the one or more lymphocytes and one or more monocytes that fits a periodic function, the controller extrapolates the fitted periodic function to a plurality of proposed future treatment dates (606). The proposed treatment dates are future dates occurring subsequent to the observed time period during which the data was collected. For example, the process may extrapolate the periodic function 5, 10, 15 or 20 days ahead of the observed time period. In this way, the process may analyze the data to identify which proposed treatment date in the near future is favorable for delivery of therapeutic treatment. In some examples, the process may extrapolate the periodic function for more or fewer days depending in part upon, for example, the periodicity of the periodic function, the number of data points obtained during the observed time period, the biological variables under analysis, the disease or condition for which the patient is being treated, the type and/or frequency of the proposed treatment, and other factors.

Based on the fitted periodic functions for each of the lymphocytes and for each of the monocytes, the controller determines a lymphocyte-to-monocyte ratio on each of the proposed treatment dates (508).

The controller may determine one or more favorable treatment date(s) based on the lymphocyte-to-monocyte ratio (610). In one example, the controller may identify date(s) where the lymphocyte-to-monocyte ratio is at a maximum. In another example, the controller may identify one or more date(s) where the lymphocyte-to-monocyte ratio for certain of the lymphocytes and/or certain of the monocytes is at a maximum. The date(s) when these conditions are met is recommended as one or more favorable date(s) to deliver the pharmacological or other therapeutic treatment to a patient (612). In recommending favorable treatment date(s) (612) the process may also establish a treatment plan for the patient based on the one or more favorable date(s) to deliver the therapeutic treatment to the patient. For example, the controller may generate a report, display, or otherwise communicate a recommendation as to the one or more favorable date(s) to deliver the treatment to the patient and/or the treatment plan for the patient based on the favorable date(s) to deliver the treatment to the patient (612). In some examples, the treatment may further be delivered (administered) to the patient on one or more of the identified favorable treatment date(s) (614).

In this manner, in one example, a method of cancer treatment may include administering chemotherapy treatment to a patient on a favorable treatment date identified based on a predicted lymphocyte-to-monocyte ratio in the blood of the patient on the favorable treatment date. In another example, a method of cancer treatment may include administering chemotherapy treatment to a patient on a favorable treatment date identified based on a predicted state of at least one lymphocyte subtype in the blood of the patient on the favorable treatment date, and on a predicted state of at least one monocyte subtype in the blood of the patient on the favorable treatment date.

### Example 1

A method of identifying one or more favorable dates to deliver a pharmacological treatment to a patient, comprising: receiving data corresponding to concentrations of one or more biological variables in blood samples from the patient over an observed time period; for each of the biological variables, fitting a periodic function to the received data corresponding to the concentration of the biological variable in the blood samples of the patient; for each of the biological variables, extrapolating the corresponding fitted periodic function to a plurality of proposed treatment dates occurring subsequent to the observed time period; for each of the biological variables, determining a state of the corresponding fitted periodic function on each of the plurality of proposed treatment dates, wherein the state on a proposed treatment date for a first set of the biological variables is determined to be favorable if the fitted periodic function is greater than a threshold value associated with the biological variable in the first set of biological variables on the proposed treatment date, and wherein the state on a proposed treatment date for a second set of the biological variables is determined to be favorable if the fitted periodic function is less than a threshold value associated with the biological variable in the second set of biological variables on the proposed treatment date; and identifying at least one of the plurality of proposed treatment dates as a favorable date to deliver the pharmacological treatment to the patient based on the determined states of each of the biological variables on each of the plurality of proposed treatment dates.

### Example 2

The method of Example 1 wherein the identified at least one favorable date to deliver the pharmacological treatment to the patient corresponds to one of the plurality of proposed treatment dates having a maximum number of the biological variables in a favorable state.

### Example 3

The method of any of Examples 1-2 wherein for each of the biological variables, determining whether the extrapolated corresponding fitted periodic function is in an unfavorable state on each of the proposed treatment dates; and wherein the identified at least one favorable date to deliver the pharmacological treatment to the patient corresponds to one of the plurality of proposed treatment dates having a maximum number of the biological variables in a favorable state and a minimum number of the biological variables in an unfavorable state.

### Example 4

The method of any of Examples 1-3 further comprising developing a treatment plan for the patient based on the identified at least one favorable date to deliver the pharmacological treatment to the patient.

### Example 5

The method of any of Examples 1-4 further comprising delivering the pharmacological treatment to the patient on the identified at least one favorable date to deliver the pharmacological treatment to the patient.

### Example 6

The method of any of Examples 1-5 wherein the first set of biological variables includes at least one lymphocyte sub-type and the second set of biological variables includes at least one monocyte sub-type.

### Example 7

The method of any of Examples 1-6 wherein the first set of the biological variables includes at least one of CD3.4 and GRO and the second set of the biological variables includes at least one of IL-2, CD123.DR(DC2), CD11c/86, CD11c/14, TGFa, and IFNg.

### Example 8

The method of any of Examples 1-7 wherein fitting a periodic function to the received data corresponding to the concentration of the biological variable in the blood samples of the patient includes fitting the received data corresponding to the concentration of the biological variable in the blood samples of the patient to a sinusoidal function.

### Example 9

A method of identifying one or more favorable dates to deliver a pharmacological treatment to a patient, comprising: receiving data corresponding to levels of one or more lymphocyte subtypes in blood samples from the patient over an observed time period; receiving data corresponding to levels of one or more monocyte subtypes in blood samples from the patient over the observed time period; for each of the lymphocyte subtypes, fitting a periodic function to the received data corresponding to the levels of the lymphocyte subtype in the blood samples of the patient, and extrapolating the fitted periodic function to a plurality of proposed treatment dates occurring subsequent to the observed time period; for each of the monocyte subtypes, fitting a periodic function to the received data corresponding to the levels of the monocyte subtype in the blood samples of the patient, and extrapolating the fitted periodic function to a plurality of proposed treatment dates occurring subsequent to the observed time period; determining a lymphocyte-to-monocyte ratio on each of the plurality of proposed treatment dates based on the extrapolated fitted periodic function for the lymphocyte subtype and the extrapolated fitted periodic function for the monocyte subtype; identifying at least one of the plurality of proposed treatment dates as a favorable date to deliver the pharmacological treatment to the patient, wherein the favorable date to deliver the pharmacological treatment to the patient corresponds to the proposed treatment date when the lymphocyte-to-monocyte ratio is at or near a maximum.

### Example 10

The method of Example 9 further comprising delivering the pharmacological treatment to the patient on the at least one identified favorable date.

### Example 11

The method of any of Examples 9-10 wherein the lymphocyte-to-monocyte ratio includes a ratio of the lymphocyte concentration to the monocyte concentration.

### Example 12

The method of any of Examples 9-11 wherein the lymphocyte-to-monocyte ratio includes a ratio of the absolute lymphocyte count to the absolute monocyte count.

### Example 13

The method of any of Examples 9-12 wherein the lymphocyte-to-monocyte ratio is based on a defined set of one or more lymphocyte subtypes and a defined set of one or more monocyte subtypes.

### Example 14

The method of any of Examples 9-13 wherein the defined set of one or more lymphocyte subtypes and the defined set of one or more monocyte subtypes are different for different types of cancers.

### Example 15

A method of identifying one or more favorable dates to deliver a pharmacological treatment to a patient, comprising: receiving data corresponding to levels of one or more lymphocyte subtypes in blood samples from the patient over an observed time period; receiving data corresponding to levels of one or more monocyte subtypes in blood samples from the patient over the observed time period; for each of the lymphocyte subtypes, fitting a periodic function to the received data corresponding to the levels of the one or more lymphocyte subtypes in the blood samples of the patient, and extrapolating the fitted periodic function to a plurality of proposed treatment dates occurring subsequent to the observed time period; for each of the monocyte subtypes, fitting a periodic function to the received data corresponding to the levels of the one or more monocyte subtypes in the blood samples of the patient, and extrapolating the fitted periodic function to the plurality of proposed treatment dates occurring subsequent to the observed time period; for each of the lymphocyte subtypes, determining a state of the extrapolated fitted periodic function on each of the plurality of proposed treatment dates, wherein the state on a proposed treatment date is determined to be favorable if the extrapolated fitted periodic function is greater than a threshold value associated with the lymphocyte subtype on the proposed treatment date; for each of the monocyte subtypes, determining a state of the extrapolated fitted periodic function on each of the plurality of proposed treatment dates, wherein the state on a proposed treatment date is determined to be favorable if the extrapolated fitted periodic function is less than a threshold value associated with the monocyte subtype on the proposed treatment date; and identifying at least one of the plurality of proposed treatment dates as a favorable date to deliver the pharmacological treatment to the patient, wherein the identified favorable date to deliver the pharmacological treatment to the patient corresponds to the proposed treatment date on which a maximum number of the lymphocyte subtypes are determined to be in a favorable state and a maximum number of the monocyte subtypes are determined to be in a favorable state.

### Example 16

The method of Example 15 further comprising developing a treatment plan for the patient based on the at least one favorable date to deliver the pharmacological treatment to the patient.

### Example 17

The method of any of Examples 15-16 further comprising delivering the pharmacological treatment to the patient on the at least one favorable date to deliver the pharmacological treatment to the patient.

### Example 18

The method of any of Example 15-17 wherein the fitted periodic functions are sinusoidal periodic functions.

### Example 19

A method of identifying one or more favorable dates to deliver a pharmacological treatment to a patient, comprising: receiving data corresponding to concentrations of one or more biological variables in blood samples from the patient over an observed time period; for each of the biological variables, fitting a periodic function to the received data corresponding to the concentration of the biological variable in the blood samples of the patient; for each of the biological variables, extrapolating the corresponding fitted periodic function to a plurality of proposed treatment dates occurring subsequent to the observed time period; for each of a first set of the one or more biological variables and on each of the plurality of proposed treatment dates, determining a state of the biological variable on the proposed treatment date, wherein the state is determined to be favorable if the corresponding fitted periodic function is greater than a corresponding threshold value; for each of a second set of the one or more biological variables and on each of the plurality of proposed treatment dates, determining a state of the biological variable on the proposed treatment date, wherein the state is determined to be favorable if the corresponding fitted periodic function is less than a corresponding threshold value; and identifying at least one of the plurality of proposed treatment dates as a favorable date to deliver the pharmacological treatment to the patient based on the determined state for each of the biological variables.

### Example 20

The method of Example 19 further comprising, for each of the biological variables, determining a threshold value based on the received data corresponding to the concentration of the biological variable in the blood samples of the patient, wherein the state of the biological variable is determined to be UP if the fitted periodic function is greater than the threshold value on a proposed treatment date, and wherein the state of the biological variable is determined to be DOWN if the fitted periodic function is less than the threshold value on a proposed treatment date.

### Example 21

The method of Example 20, wherein if the biological variable is a lymphocyte subtype, the state of the biological variable is favorable if the state is determined to be UP on the proposed treatment date, and wherein if the biological variable is a monocyte subtype, the state of the biological variable is favorable if the state is determined to be DOWN on the proposed treatment date.

### Example 22

A method of cancer treatment, comprising administering chemotherapy treatment to a patient on a favorable treatment date identified based on a predicted lymphocyte-to-monocyte ratio in the blood of the patient on the favorable treatment date.

### Example 23

A method of cancer treatment, comprising administering chemotherapy treatment to a patient on a favorable treatment date identified based on a predicted state of at least one lymphocyte subtype in the blood of the patient on the favorable treatment date, and on a predicted state of at least one monocyte subtype in the blood of the patient on the favorable treatment date.

### Example 24

The method of Example 23, wherein the predicted state of the at least one lymphocyte subtype is favorable if a value of a periodic function fitted to concentration values of the at least one lymphocyte subtype in the blood of the patient over an observed period of time and extrapolated to a proposed treatment date is greater than a first threshold value on the proposed treatment date, and wherein the predicted state of the at least one monocyte subtype is favorable if a value of a periodic function fitted to concentration values of the at least one monocyte subtype in the blood of the patient over an observed period of time and extrapolated to a proposed treatment date is less than a second threshold value on the proposed treatment date.

The techniques described in this disclosure, including functions performed by a processor, controller, control unit, or control system, may be implemented within one or more of a general purpose microprocessor, digital signal processor (DSP), application specific integrated circuit (ASIC), field programmable gate array (FPGA), programmable logic devices (PLDs), or other equivalent logic devices. Accordingly, the terms “processor” “processing unit” or “controller,” as used herein, may refer to any one or more of the foregoing structures or any other structure suitable for implementation of the techniques described herein.

The various components illustrated herein may be realized by any suitable combination of hardware, firmware, and/or software. In the figures, various components are depicted as separate units or modules. However, all or several of the various components described with reference to these figures may be integrated into combined units or modules within common hardware, firmware, and/or software. Accordingly, the representation of features as components, units or modules is intended to highlight particular functional features for ease of illustration, and does not necessarily require realization of such features by separate hardware, firmware, or software components. In some cases, various units may be implemented as programmable processes performed by one or more processors or controllers.

Any features described herein as modules, devices, or components may be implemented together in an integrated logic device or separately as discrete but interoperable logic devices. In various aspects, such components may be formed at least in part as one or more integrated circuit devices, which may be referred to collectively as an integrated circuit device, such as an integrated circuit chip or chipset. Such circuitry may be provided in a single integrated circuit chip device or in multiple, interoperable integrated circuit chip devices, and may be used in any of a variety of applications and devices.

If implemented in part by software, the techniques may be realized at least in part by a computer-readable data storage medium comprising code with instructions that, when executed by one or more processors or controllers, performs one or more of the methods described in this disclosure. The computer-readable storage medium may form part of a computer program product, which may include packaging materials. The computer-readable medium may comprise random access memory (RAM) such as synchronous dynamic random access memory (SDRAM), read-only memory (ROM), non-volatile random access memory (NVRAM), electrically erasable programmable read-only memory (EEPROM), embedded dynamic random access memory (eDRAM), static random access memory (SRAM), flash memory, magnetic or optical data storage media. Any software that is utilized may be executed by one or more processors, such as one or more DSP's, general purpose microprocessors, ASIC's, FPGA's, or other equivalent integrated or discrete logic circuitry.

Various examples have been described. These and other examples are within the scope of the following claims.

1. A method of identifying one or more favorable dates to deliver a pharmacological treatment to a patient, comprising:
receiving data corresponding to concentrations of one or more biological variables in blood samples from the patient over an observed time period;
for each of the biological variables, fitting a periodic function to the received data corresponding to the concentration of the biological variable in the blood samples of the patient;
for each of the biological variables, extrapolating the corresponding fitted periodic function to a plurality of proposed treatment dates occurring subsequent to the observed time period;
for each of the biological variables, determining a state of the corresponding fitted periodic function on each of the plurality of proposed treatment dates, wherein the state on a proposed treatment date for a first set of the biological variables is determined to be favorable if the fitted periodic function is greater than a threshold value associated with the biological variable in the first set of biological variables on the proposed treatment date, and wherein the state on a proposed treatment date for a second set of the biological variables is determined to be favorable if the fitted periodic function is less than a threshold value associated with the biological variable in the second set of biological variables on the proposed treatment date; and
identifying at least one of the plurality of proposed treatment dates as a favorable date to deliver the pharmacological treatment to the patient based on the determined states of each of the biological variables on each of the plurality of proposed treatment dates.

2. The method of claim 1 wherein the identified at least one favorable date to deliver the pharmacological treatment to the patient corresponds to one of the plurality of proposed treatment dates having a maximum number of the biological variables in a favorable state.

3. The method of claim 1 wherein for each of the biological variables, determining whether the extrapolated corresponding fitted periodic function is in an unfavorable state on each of the proposed treatment dates; and wherein the identified at least one favorable date to deliver the pharmacological treatment to the patient corresponds to one of the plurality of proposed treatment dates having a maximum number of the biological variables in a favorable state and a minimum number of the biological variables in an unfavorable state.

4. The method of claim 1 further comprising developing a treatment plan for the patient based on the identified at least one favorable date to deliver the pharmacological treatment to the patient.

5. The method of claim 1 further comprising delivering the pharmacological treatment to the patient on the identified at least one favorable date to deliver the pharmacological treatment to the patient.

6. The method of claim 1 wherein the first set of biological variables includes at least one lymphocyte sub-type and the second set of biological variables includes at least one monocyte sub-type.

7. The method of claim 1 wherein the first set of the biological variables includes at least one of CD3.4 and GRO and the second set of the biological variables includes at least one of IL-2, CD123.DR(DC2), CD11c/86, CD11c/14, TGFa, and IFNg.

8. The method of claim 1 wherein fitting a periodic function to the received data corresponding to the concentration of the biological variable in the blood samples of the patient includes fitting the received data corresponding to the concentration of the biological variable in the blood samples of the patient to a sinusoidal function.

9. A method of identifying one or more favorable dates to deliver a pharmacological treatment to a patient, comprising:
receiving data corresponding to levels of one or more lymphocyte subtypes in blood samples from the patient over an observed time period;
receiving data corresponding to levels of one or more monocyte subtypes in blood samples from the patient over the observed time period;
for each of the lymphocyte subtypes, fitting a periodic function to the received data corresponding to the levels of the lymphocyte subtype in the blood samples of the patient, and extrapolating the fitted periodic function to a plurality of proposed treatment dates occurring subsequent to the observed time period;
for each of the monocyte subtypes, fitting a periodic function to the received data corresponding to the levels of the monocyte subtype in the blood samples of the patient, and extrapolating the fitted periodic function to a plurality of proposed treatment dates occurring subsequent to the observed time period;
determining a lymphocyte-to-monocyte ratio on each of the plurality of proposed treatment dates based on the extrapolated fitted periodic function for the lymphocyte subtype and the extrapolated fitted periodic function for the monocyte subtype;
identifying at least one of the plurality of proposed treatment dates as a favorable date to deliver the pharmacological treatment to the patient, wherein the favorable date to deliver the pharmacological treatment to the patient corresponds to the proposed treatment date when the lymphocyte-to-monocyte ratio is at or near a maximum.

10. The method of claim 9 further comprising delivering the pharmacological treatment to the patient on the at least one identified favorable date.

11. The method of claim 9 wherein the lymphocyte-to-monocyte ratio includes a ratio of the lymphocyte concentration to the monocyte concentration.

12. The method of claim 9 wherein the lymphocyte-to-monocyte ratio includes a ratio of the absolute lymphocyte count to the absolute monocyte count.

13. The method of claim 9 wherein the lymphocyte-to-monocyte ratio is based on a defined set of one or more lymphocyte subtypes and a defined set of one or more monocyte subtypes.

14. The method of claim 13 wherein the defined set of one or more lymphocyte subtypes and the defined set of one or more monocyte subtypes are different for different types of cancers.

15. A method of identifying one or more favorable dates to deliver a pharmacological treatment to a patient, comprising:
receiving data corresponding to levels of one or more lymphocyte subtypes in blood samples from the patient over an observed time period;
receiving data corresponding to levels of one or more monocyte subtypes in blood samples from the patient over the observed time period;
for each of the lymphocyte subtypes, fitting a periodic function to the received data corresponding to the levels of the one or more lymphocyte subtypes in the blood samples of the patient, and extrapolating the fitted periodic function to a plurality of proposed treatment dates occurring subsequent to the observed time period;
for each of the monocyte subtypes, fitting a periodic function to the received data corresponding to the levels of the one or more monocyte subtypes in the blood samples of the patient, and extrapolating the fitted periodic function to the plurality of proposed treatment dates occurring subsequent to the observed time period;
for each of the lymphocyte subtypes, determining a state of the extrapolated fitted periodic function on each of the plurality of proposed treatment dates, wherein the state on a proposed treatment date is determined to be favorable if the extrapolated fitted periodic function is greater than a threshold value associated with the lymphocyte subtype on the proposed treatment date;
for each of the monocyte subtypes, determining a state of the extrapolated fitted periodic function on each of the plurality of proposed treatment dates, wherein the state on a proposed treatment date is determined to be favorable if the extrapolated fitted periodic function is less than a threshold value associated with the monocyte subtype on the proposed treatment date; and
identifying at least one of the plurality of proposed treatment dates as a favorable date to deliver the pharmacological treatment to the patient, wherein the identified favorable date to deliver the pharmacological treatment to the patient corresponds to the proposed treatment date on which a maximum number of the lymphocyte subtypes are determined to be in a favorable state and a maximum number of the monocyte subtypes are determined to be in a favorable state.

16. The method of claim 15 further comprising developing a treatment plan for the patient based on the at least one favorable date to deliver the pharmacological treatment to the patient.

17. The method of claim 15 further comprising delivering the pharmacological treatment to the patient on the at least one favorable date to deliver the pharmacological treatment to the patient.

18. The method of claim 15 wherein the fitted periodic functions are sinusoidal periodic functions.

19. A method of identifying one or more favorable dates to deliver a pharmacological treatment to a patient, comprising:
receiving data corresponding to concentrations of one or more biological variables in blood samples from the patient over an observed time period;
for each of the biological variables, fitting a periodic function to the received data corresponding to the concentration of the biological variable in the blood samples of the patient;
for each of the biological variables, extrapolating the corresponding fitted periodic function to a plurality of proposed treatment dates occurring subsequent to the observed time period;
for each of a first set of the one or more biological variables and on each of the plurality of proposed treatment dates, determining a state of the biological variable on the proposed treatment date, wherein the state is determined to be favorable if the corresponding fitted periodic function is greater than a corresponding threshold value;
for each of a second set of the one or more biological variables and on each of the plurality of proposed treatment dates, determining a state of the biological variable on the proposed treatment date, wherein the state is determined to be favorable if the corresponding fitted periodic function is less than a corresponding threshold value; and
identifying at least one of the plurality of proposed treatment dates as a favorable date to deliver the pharmacological treatment to the patient based on the determined state for each of the biological variables.

20. The method of claim 19 further comprising, for each of the biological variables, determining a threshold value based on the received data corresponding to the concentration of the biological variable in the blood samples of the patient, wherein the state of the biological variable is determined to be UP if the fitted periodic function is greater than the threshold value on a proposed treatment date, and wherein the state of the biological variable is determined to be DOWN if the fitted periodic function is less than the threshold value on a proposed treatment date.

21. The method of claim 20, wherein if the biological variable is a lymphocyte subtype, the state of the biological variable is favorable if the state is determined to be UP on the proposed treatment date, and wherein if the biological variable is a monocyte subtype, the state of the biological variable is favorable if the state is determined to be DOWN on the proposed treatment date.

22. A method of cancer treatment, comprising administering chemotherapy treatment to a patient on a favorable treatment date identified based on a predicted lymphocyte-to-monocyte ratio in the blood of the patient on the favorable treatment date.

23. A method of cancer treatment, comprising administering chemotherapy treatment to a patient on a favorable treatment date identified based on a predicted state of at least one lymphocyte subtype in the blood of the patient on the favorable treatment date, and on a predicted state of at least one monocyte subtype in the blood of the patient on the favorable treatment date.

24. The method of claim 23, wherein the predicted state of the at least one lymphocyte subtype is favorable if a value of a periodic function fitted to concentration values of the at least one lymphocyte subtype in the blood of the patient over an observed period of time and extrapolated to a proposed treatment date is greater than a first threshold value on the proposed treatment date, and wherein the predicted state of the at least one monocyte subtype is favorable if a value of a periodic function fitted to concentration values of the at least one monocyte subtype in the blood of the patient over an observed period of time and extrapolated to a proposed treatment date is less than a second threshold value on the proposed treatment date.
